(19) United States
(12) Patent Application Publication
(10) Pub. No.: US 2026/0229337 A1
(43) Pub. Date: Aug. 6, 2026

(54) Methods to Assess Clinical Outcome Based Upon Updated Probabilities and Treatments Thereof

(71) Applicant: The Board of Trustees of the Leland Stanford Junior University, Stanford, CA (US)

(72) Inventors: David M. Kurtz, San Carlos, CA (US); Arash Ash Alizadeh, San Mateo, CA (US); Maximilian Diehn, San Carlos, CA (US); Mohammad Shahrokh Esfahani, Mountain View, CA (US)

(73) Assignee: The Board of Trustees of the Leland Stanford Junior University, Stanford, CA (US)

(21) Appl. No.: 19/457,011

(22) Filed: Jan. 22, 2026

Related U.S. Application Data
(63) Continuation of application No. 17/646,621, filed on Dec. 30, 2021, now abandoned.
Application No. 17/646,621 is a continuation of application No. PCT/US2020/040936, filed on Jul. 6, 2020.
(60) Provisional application No. 62/870,411, filed on Jul. 3, 2019.

(51) Int. Cl. G16H 20/40; A61B 5/00; G16H 50/20

(52) U.S. Cl. CPC G16H 20/40; A61B 5/4848; G16H 50/20

(57) ABSTRACT

Methods of treatment based on a prognosis as determined utilizing a Bayesian framework are provided. Clinical data is utilized within a Bayesian framework to obtain a prognosis of a medical disorder. A prognosis can be updated utilizing a Bayesian framework when subsequent clinical data is acquired, such as clinical data acquired during a treatment or clinical monitoring.

### CROSS REFERENCE TO RELATED APPLICATIONS

This application is a continuation of U.S. patent application Ser. No. 17/646,621, entitled “Methods to Assess Clinical Outcome Based Upon Updated Probabilities and Treatments Thereof” to David M. Kurtz et al., filed Dec. 30, 2021, which is a continuation of PCT Application Ser. No. PCT/US2020/040936, entitled “Methods to Assess Clinical Outcome Based Upon Updated Probabilities and Treatments Thereof” to David M. Kurtz et al., filed Jul. 6, 2020, which claims priority to U.S. Provisional Application Ser. No. 62/870,411, entitled “Methods of Treatments Based Upon Updated Probabilities of Clinical Outcome” to David M. Kurtz et al., filed Jul. 3, 2019, the disclosures of which are incorporated herein by reference in their entireties.

### STATEMENT REGARDING FEDERALLY SPONSORED RESEARCH OR DEVELOPMENT

This invention was made with Government support under contracts CA186569 and CA188298 awarded by the National Institutes of Health. The Government has certain rights in the invention.

### TECHNICAL FIELD

The invention is generally directed to methods involving diagnostics and treatments, and more specifically to diagnostics and treatments based upon updated probabilities of an individual's clinical outcome.

### BACKGROUND

Biological and clinical heterogeneity between patients remain inherent barriers to improving disease outcomes. Such variation contributes to dramatically different outcomes for patients nominally sharing the same disease. Over the past five decades, significant efforts have been made in unraveling this heterogeneity between patients through refined classifications that capture physical, anatomical, radiographic, histological, and molecular features of the disease. For example, in the field of cancer, by identifying patients with similar clinical phenotypes, anatomic staging systems have enabled more uniform treatment practices, leading to improvement in outcomes for patients (see S. B. Edge and C. C. Compton, Annals of surgical oncology 17, 1471-1474 (2010); W. J. Gradishar, et al., Journal of the National Comprehensive Cancer Network: JNCCN 16, 310-320 (2018); S. M. Horwitz, et al., Journal of the National Comprehensive Cancer Network: JNCCN 14, 1067-1079 (2016); and W. G. Wierda, et al., Journal of the National Comprehensive Cancer Network: JNCCN 15, 293-311 (2017)). Similarly, molecular dissection of differing biological features has resulted in the identification of targetable subtypes in diverse tumors, including HER2 amplifications in breast cancer and EGFR mutations in lung cancers, also leading to improved patient outcomes (see T. J. Lynch, et al., The New England journal of medicine 350, 2129-2139 (2004); J. G. Paez, et al., Science 304, 1497-1500 (2004); M. J. Pencina, R. B. D'Agostino, and R. S. Vasan, Clin Chem Lab Med 48, 1703-1711 (2005); E. H. Romond, et al., The New England journal of medicine 353, 1673-1684 (2005); and D. J. Slamon, et al., Science 235, 177-182 (1987)). Despite such advances, however, significant heterogeneity remains in most cancer subtypes (see P. L. Bedard, et al., Nature 501, 355-364 (2013)). Heterogeneity is common within many other diseases as well, including cardiovascular disease. As such, there is a need to individualize clinical course of diagnostics and treatments.

### SUMMARY

Various embodiments are directed to diagnostics and treatments utilizing naïve Bayes or Bayesian framework. In various embodiments, a set of clinical data and a naïve Bayes or Bayesian framework are utilized to determine a clinical assessment. In various embodiments, a clinical assessment is updated with subsequent set(s) of clinical data and the naïve Bayes or Bayesian framework.

In an embodiment, a method is for personalized clinical assessment of an individual having a medical disorder. The method obtains a naïve Bayes or a Bayesian framework built to provide a clinical assessment of a medical disorder based upon sets of clinical data. The method obtains an initial set of clinical data of an individual. Utilizing the naïve Bayes or the Bayesian framework and the individual's initial set of clinical data, the method determines an initial clinical assessment. Based upon the initial clinical assessment, the method administers an initial course of treatment to the individual. The method obtains a subsequent set of clinical data of the individual. Utilizing the naïve Bayes or the Bayesian framework and the individual's initial and subsequent sets of clinical data, the method determines a subsequent clinical assessment. Based upon the subsequent clinical assessment, the method administers a subsequent course of treatment to the individual.

In another embodiment, the method further obtains an additional subsequent set of clinical data of the individual. Utilizing the naïve Bayes or the Bayesian framework and the individual's initial, subsequent, and additional subsequent sets of clinical data, the method further determines an additional subsequent clinical assessment. Based upon the additional subsequent clinical assessment, the method further administers an additional subsequent course of treatment to the individual.

In yet another embodiment, the disorder is a cancer.

In a further embodiment, the cancer is one of: diffuse large B-cell lymphoma (DLBCL), chronic lymphocytic leukemia (CLL), or breast adenocarcinoma (BRCA).

In yet a further embodiment, the cancer is diffuse large B-cell lymphoma (DLBCL) and the initial set of clinical data includes at least one of: international prognostic index, molecular cell of origin, quantity of initial circulating tumor DNA, or a medical image scan.

In an even further embodiment, the cancer is chronic lymphocytic leukemia (CLL) and the initial set of clinical data includes at least one of: first line of therapy or international prognostic index.

In yet an even further embodiment, the cancer is breast adenocarcinoma (BRCA) and the initial set of clinical data includes at least one of: clinical stage, tumor grade, or status of estrogen receptor (ER) and human epidermal growth factor receptor 2 (HER2).

In still yet an even further embodiment, the cancer is non-small cell lung cancer (NSCLC) and the initial set of clinical data includes at least one of: gross tumor volume, KEAP1 mutational status, or histology.

In still yet an even further embodiment, the cancer is diffuse large B-cell lymphoma (DLBCL) and the subsequent set or the additional subsequent set of clinical data includes at least one of: quantity of circulating tumor DNA or a medical image scan.

In still yet an even further embodiment, the cancer is chronic lymphocytic leukemia (CLL) and the subsequent set or the additional subsequent set of clinical data includes minimal residual disease.

In still yet an even further embodiment, the cancer is breast adenocarcinoma (BRCA) and the subsequent set or the additional subsequent set of clinical data includes pathological response to therapy.

In still yet an even further embodiment, the cancer is non-small cell lung cancer (NSCLC) and the subsequent clinical data includes ctDNA molecular residual disease.

In still yet an even further embodiment, the cancer is diffuse large B-cell lymphoma (DLBCL) and the clinical assessment indicates event free survival.

In still yet an even further embodiment, the cancer is diffuse large B-cell lymphoma (DLBCL) and the clinical assessment indicates overall survival.

In still yet an even further embodiment, the cancer is chronic lymphocytic leukemia (CLL) and the clinical assessment indicates progression free survival.

In still yet an even further embodiment, the cancer is breast adenocarcinoma (BRCA) and the clinical assessment indicates distant relapse free survival.

In still yet an even further embodiment, the cancer is non-small cell lung cancer (NSCLC) and the clinical assessment indicates progression free survival.

In still yet an even further embodiment, the disorder is diabetes mellitus and the initial set of clinical data includes at least one of: age, type of diabetes, fasting blood glucose, hemoglobin A1C, or comorbidities.

In still yet an even further embodiment, the disorder is sepsis and the initial set of clinical data includes at least one of: blood pressure, heart rate, temperature, respiratory rate, oxygenation status, or blood counts.

In still yet an even further embodiment, the disorder is diabetes mellitus and the subsequent clinical data includes at least one of: serial fasting blood glucose measurements or hemoglobin A1C measurements.

In still yet an even further embodiment, the disorder is sepsis and the subsequent clinical data includes at least one of: blood culture results, serial blood pressure measurements, heart rate, temperature, respiratory rate, oxygenation status, or blood counts.

In still yet an even further embodiment, the naïve Bayes framework is utilized to determine a clinical assessment at particular endpoint post initial course of treatment.

In still yet an even further embodiment, the Bayesian framework is utilized and incorporates Cox proportional hazard.

In still yet an even further embodiment, the initial course of treatment is the standard of care.

In still yet an even further embodiment, the initial clinical assessment is unfavorable and the initial course of treatment is more aggressive than the standard of care.

In still yet an even further embodiment, the initial clinical assessment is favorable and the initial course of treatment is more aggressive than the standard of care.

In still yet an even further embodiment, the subsequent clinical assessment is the same as the initial clinical assessment and the subsequent course of treatment maintains the initial course of treatment.

In still yet an even further embodiment, the subsequent clinical assessment is less favorable than the initial clinical assessment and the subsequent course of treatment is more aggressive than the initial course of treatment.

In still yet an even further embodiment, the subsequent clinical assessment is more favorable than the initial clinical assessment and the subsequent course of treatment is less aggressive than the initial course of treatment.

In still yet an even further embodiment, the additional subsequent clinical assessment is the same as the subsequent clinical assessment and the additional subsequent course of treatment maintains the subsequent course of treatment.

In still yet an even further embodiment, the additional subsequent clinical assessment is less favorable than the subsequent clinical assessment and the additional subsequent course of treatment is more aggressive than the subsequent course of treatment.

In still yet an even further embodiment, the additional subsequent clinical assessment is more favorable than the subsequent clinical assessment and the additional subsequent course of treatment is less aggressive than the subsequent course of treatment.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a flow diagram of a method to treat an individual and to update treatment based upon risk factors in accordance with various embodiments.

FIG. 2 provides a schema for design and motivation for development of the Continuous Individualized Risk Index in accordance with various embodiments.

FIG. 3 provides a graphical schema depicting CIRI-DLBCL in accordance with various embodiments.

FIGS. 4A and 4B provide data graphs depicting data parameters for CIRI-DLBCL, fixed endpoint, utilized in accordance with various embodiments.

FIG. 5A provides Table 1: Patient and cohort data for CIRI-DLBCL, utilized in accordance with various embodiments.

FIG. 5B provides Table 2: Parameters for CIRI-DLBCL, utilized in accordance with various embodiments.

FIG. 6 provides a data graph depicting calibration of CIRI-DLBCL, utilized in accordance with various embodiments.

FIG. 7A provides a bar plot demonstrating the C-Statistic and 95% C.I. for predicting EFS24 by the IPI, all pretreatment factors, EMR, MMR, interim PET/CT, and CIRI, generated in accordance with various embodiments. Predictions from CIRI-DLBCL are made after integration of all data; * indicates P<0.05.

FIG. 7B provides a bar plot demonstrating the C-Statistic and 95% C.I. for predicting OS24 by the IPI, all pretreatment factors, EMR, MMR, interim PET/CT, and CIRI, generated in accordance with various embodiments. Predictions from CIRI-DLBCL are made after integration of all data; * indicates P<0.05.

FIGS. 8A and 8B provide data graphs depicting data parameters for CIRI-DLBCL, survival analysis, utilized in accordance with various embodiments.

FIG. 9 provides a graphical schema depicting CIRI-DLBCL incorporating Bayesian proportional hazards in accordance with various embodiments.

FIG. 10 provides a data graph depicting calibration of CIRI-DLBCL incorporating Bayesian proportional hazards, utilized in accordance with various embodiments.

FIG. 11 provides data graphs depicting the calibration of CIRI-DLBCL survival analysis compared with event-free survival endpoints from 12 months to 36 months, utilized in accordance with various embodiments.

FIG. 12 provides bar plots depicting the C-Statistic and 95% C.I. for predicting EFS at multiple time intervals by the IPI, all pretreatment risk factors, EMR, MMR, interim PET/CT, and CIRI, generated in accordance with various embodiments. Predictions from CIRI-DLBCL are made after integration of all data; * indicates P<0.05.

FIG. 13 provides Kaplan-Meir estimates for event-free survival for patients stratified by CIRI-DLBCL into three groups after cycle four and across all time points, generated in accordance with various embodiments.

FIG. 14 provides bar plots depicting the C-Statistic and 95% C.I. for predicting OS at multiple time intervals by the IPI, all pretreatment risk factors, EMR, MMR, interim PET/CT, and CIRI, generated in accordance with various embodiments. Predictions from CIRI-DLBCL are made after integration of all data; * indicates P<0.05.

FIG. 15 provides Kaplan-Meir estimates for overall survival for patients stratified by CIRI-DLBCL into three groups after cycle four and across all time points, generated in accordance with various embodiments.

FIG. 16 provides a graphical schema depicting CIRI-CLL timeline in accordance with various embodiments.

FIG. 17 provides Table 3: Patient and cohort data for CIRI-CLL, utilized in accordance with various embodiments.

FIGS. 18A and 18B provide data graphs depicting parameter determination for various risk factors in CLL for use in the CIRI-CLL, survival analysis, utilized in accordance with various embodiments.

FIG. 19 provides a data graph depicting calibration of CIRI-CLL survival analysis, utilized in accordance with various embodiments.

FIG. 20 provides data graphs depicting the calibration of CIRI-CLL survival analysis compared with event-free survival endpoints from 12 months to 60 months, utilized in accordance with various embodiments.

FIG. 21 provides bar plots depicting the C-Statistic and 95% C.I. for predicting progression-free survival at various time-points using the CLL-IPI, all pretreatment risk factors, interim minimal residual disease (MRD), end of therapy MRD, and CIRI-CLL, generated in accordance with various embodiments. Predictions from CIRI-CLL are made after integration of all data; * indicates P<0.05.

FIG. 22 provides Kaplan-Meir estimates for PFS for patients stratified by CIRI-CLL into groups at the end of therapy and across all time points, generated in accordance with various embodiments.

FIG. 23 provides bar plots depicting the C-Statistic and 95% C.I. for predicting OS at various time-points using the CLL-IPI, all pretreatment risk factors, interim MRD, end of therapy MRD, and CIRI, generated in accordance with various embodiments. Predictions from CIRI-CLL are made after integration of all data; * indicates P<0.05.

FIG. 24 provides Kaplan-Meir estimates for overall survival for patients stratified by CIRI-CLL into groups at the end of therapy and across all time points, generated in accordance with various embodiments.

FIG. 25 provides a graphical schema depicting CIRI-BRCA timeline in accordance with various embodiments.

FIGS. 26A and 26B provide data graphs depicting parameter determination for various risk factors in BRCA for use in the CIRI-BRCA, survival analysis, utilized in accordance with various embodiments.

FIG. 27 provides Table 4: Patient and cohort data for CIRI-BRCA, utilized in accordance with various embodiments.

FIG. 28 provides a data graph depicting calibration of CIRI-BRCA survival analysis, utilized in accordance with various embodiments.

FIGS. 29A and 29B provides data graphs depicting the calibration of CIRI-BRCA survival analysis compared with event-free survival endpoints from 12 months to 60 months, utilized in accordance with various embodiments.

FIG. 30 provides bar plots depicting the C-Statistic and 95% C.I. for predicting distant-relapse-free survival at various time-points using the all pretreatment risk factors, pathologic response, and CIRI-BRCA, generated in accordance with various embodiments. Predictions from CIRI-BRCA are made after integration of all data; indicates P<0.05.

FIG. 31 provides Kaplan-Meir estimates for DRFS for patients stratified by CIRI-BRCA into groups at post surgery and across all time points, generated in accordance with various embodiments.

FIG. 32 provides data plots of the effect of increasing correlation on discrimination of outcomes by C-Statistic (Panel A), calibration intercept (Panel B), and calibration slope, generated in accordance with various embodiments.

FIG. 33 provides data plots to compare CIRI with Cox proportional hazard models by training Cox proportional hazard models starting from 20 to 200 cases drawn randomly from the validation set in CLL, generated in accordance with various embodiments.

FIG. 34 provides data plots to compare CIRI with Cox proportional hazard models by training Cox proportional hazard models starting from 20 to 200 cases drawn randomly from the validation set in BRCA, generated in accordance with various embodiments.

FIG. 35 provides a graphical schema depicting the use of interim MRD to guide therapy in CLL in accordance with various embodiments.

FIG. 36 provides data graphs depicting Kaplan-Meier estimates that show the benefit of therapy with FCR vs alternative therapies for progression-free survival in interim MRD negative patients (top panel) and interim MRD positive patients (bottom panel), generated in accordance with various embodiments.

FIG. 37 provides a graphical schema depicting the use of CIRI-CLL to guide therapy in accordance with various embodiments.

FIG. 38 provides data plots depicting the predicted benefit of FCR vs alternative immunochemotherapy for subsets of patients defined as “high” or “low” risk by various CIRI-CLL thresholds, generated in accordance with various embodiments.

FIG. 39 provides data graphs depicting Kaplan-Meier estimates that show the PFS of patients receiving FCR vs alternative therapies in patients with CIRI risk <20% (top panel) and patients with CIRI risk >20% (bottom panel), generated in accordance with various embodiments.

FIG. 40 provides a graphical schema depicting the use of pathological response to guide therapy in accordance with various embodiments.

FIG. 41 provides data graphs depicting Kaplan-Meier estimates that show the benefit of neoadjuvant therapy containing Trastuzumab+Pertuzumab vs standard therapy for disease-free survival in patients achieving a pathological CR (top panel) or not achieving a pathological CR (bottom panel), generated in accordance with various embodiments.

FIG. 42 provides a graphical schema depicting the use of CIRI-BRCA to guide therapy in accordance with various embodiments.

FIG. 43 provides data graphs depicting Kaplan-Meier estimates that show the DFS of patients receiving Trastuzumab+Pertuzumab vs standard therapy in patients with CIRI risk <15% (top panel) and patients with CIRI risk >15% (lower panel).

FIG. 44 provides data plots depicting the predicted benefit Trastuzumab+Pertuzumab containing neoadjuvant therapy for subsets of patients defined as “high” or “low” risk by various CIRI-BRCA thresholds, generated in accordance with various embodiments.

FIGS. 45 and 46 each provides an example of a CIRI-DLBCL risk profile that predicts the probability of EFS24 for a specific individual patient (DLBCL103), generated in accordance with various embodiments.

FIG. 47 provides a Table 5: P-values for Schoenfeld residuals for CIRI, utilized in accordance with various embodiments.

FIG. 48 provides an example of a probability density function comparing outcomes with frontline and salvage therapy for a specific individual (DLBCL103), generated in accordance with various embodiments.

FIG. 49 provides data graphs of predictive features associated with progression-free survival (PFS), utilized in accordance with various embodiments.

FIG. 50 provides a data graph showing the correlation between largest lesion metabolic tumor volume (MTV) and largest lesion gross tumor volume (GTV), utilized in accordance with various embodiments.

FIGS. 51 and 52 provide data graphs showing correlation of gene mutations with PFS, utilized in accordance with various embodiments.

FIG. 53 provides a data graph showing site of first progression in various tumor types, utilized in accordance with various embodiments.

FIG. 54 provides a graphical schema depicting CIRI-NSCLC timeline in accordance with various embodiments.

FIG. 55 provides bar plots depicting the C-Statistic and 95% C.I. for predicting progression free survival at various time points using KEAP1 mutation status, GTV, histology, CRT ctDNA molecular residual disease, and CIRI-NSCLC, generated in accordance with various embodiments. Predictions from CIRI-NSCLC are made after integration of all data; * indicates P<0.05.

FIGS. 56 and 57 provide Kaplan-Meir estimates for PFS for patients stratified by CIRI-NSCLS into groups across all time points, generated in accordance with various embodiments.

FIG. 58 provides a data graph depicting calibration of CIRI-NSCLC survival analysis, utilized in accordance with various embodiments.

FIG. 59 provides data graphs of CIRI-NSCLC predicted PFS and radiographic images of disease progression of two patients (LUP810 and LUMP235), generated in accordance with various embodiments.

FIG. 60 provides bar plots depicting the C-Statistic and 95% C.I. for predicting progression free survival at various time points using ctDNA molecular residual disease and CIRI-NSCLC, generated in accordance with various embodiments; * indicates P<0.05.

FIG. 61 provides Kaplan-Meir estimates for PFS for patients stratified by CIRI-NSCLS or ctDNA molecular residual disease, generated in accordance with various embodiments.

FIG. 62 illustrate the ability of CIRI-NSCLC to provide an earlier predictor of PFS than ctDNA molecular residual disease in accordance with various embodiments.

### DETAILED DESCRIPTION

Turning now to the figures and data, various methods and systems for clinical personalized assessment of medical disorders and treatments of an individual, in accordance with various embodiments, are described. In several embodiments, an individual having a medical condition is assessed by collecting clinical data from the individual over time, prior to and during treatment, such that a predicted clinical outcome is updated as new clinical data is obtained. In many embodiments, obtained clinical data is utilized within a constructed naïve Bayes or Bayesian framework to determine the individual's likely clinical outcome, which can inform an appropriate treatment for the individual. Accordingly, in numerous embodiments, an initial set of clinical data is obtained from an individual and entered into a naïve Bayes or Bayesian framework to determine an initial prediction of clinical outcome, which can be utilized to determine an initial course of treatment. As an individual's treatment and/or disorder progresses, in accordance with numerous embodiments, an intermediate set of clinical data is obtained and the initial and intermediate sets of clinical data entered into the naïve Bayes or Bayesian framework to update the prediction of clinical outcome, which can be utilized to update the course of treatment course. In numerous embodiments, as further intermediate sets of clinical data are is obtained and added to the naïve Bayes or Bayesian framework, the prediction of clinical outcome is further updated, which can be utilized to further update the course of treatment. Accordingly, in numerous embodiments, the prediction of clinical outcome of the individual is sequentially updated for each set of clinical data that is obtained and entered into the naïve Bayes or Bayesian framework, and thus the individual's course of treatment can be sequentially updated based on the updated prediction of clinical outcome.

### Clinical Assessment Utilizing a Naïve Bayes or Bayesian Framework

A number of embodiments are directed towards determining a personal clinical assessment or prognosis for an individual's medical disorder, which can be used to determine a course of treatment for the individual. In several embodiments, a personal clinical assessment is determined utilizing a naïve Bayes or Bayesian framework and the individual's personal clinical data. In some embodiments, a naïve Bayes framework is utilized to determine an individual's likelihood of a clinical endpoint (e.g., 24 months of survival). In some embodiments, a Bayesian framework is combined with a Cox proportional hazard model to determine an individual's continuous survival function over time.

In various embodiments, the personal clinical assessment determined by a naïve Bayes or Bayesian framework is updated when further clinical data (i.e., intermediate clinical data) is obtained and entered into the framework. In several embodiments, intermediate clinical data is obtained after the initial prognosis, during treatment, and/or during clinical monitoring. Accordingly, in a number of embodiments, when a clinical assessment is updated, the course of treatment can be updated based on the updated assessment. In some embodiments, a naïve Bayes or Bayesian framework also incorporates data regarding the benefit of various courses of treatment such that the clinical assessment also considers a particular course of treatment. Accordingly, in some embodiments, a particular course of treatment is entered into a naïve Bayes or Bayesian framework resulting in an improved clinical assessment, and that particular course of treatment is then administered the individual.

In several embodiments, the medical disorder to be assessed is a cancer, such as (for example) diffuse large B-cell lymphoma (DLBCL), chronic lymphocytic leukemia (CLL), breast adenocarcinoma (BRCA), non-small cell lung cancer (NSCLC), or other solid or hematologic cancers. In some embodiments, the medical disorder to be assessed is a chronic disease, such as (for example), hypertension, diabetes mellitus (DM), congestive heart failure (CHF), or chronic kidney disease (CKD). In some embodiments, the medical disorder to be assessed is an acute disease requiring hospitalization, such as infectious processes (e.g., bacterial infections, viral infections, or sepsis). It is to be noted that any medical disorder could be assessed utilizing the various embodiments described herein, especially when the disorder can utilize multiple sets of clinical data that dynamically evolve over time to determine a prognosis.

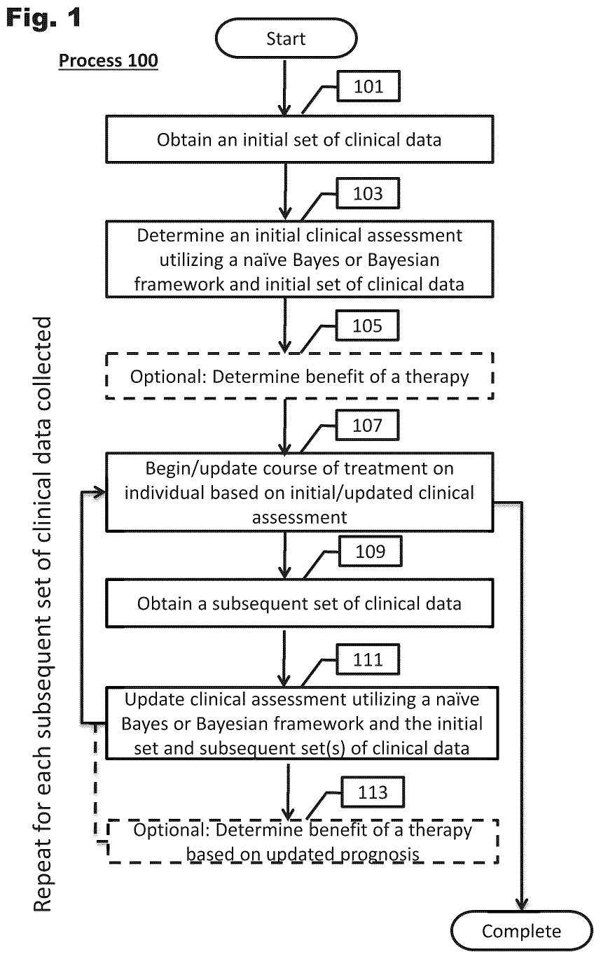

Provided in FIG. 1 is a method to assess an individual based on a prognosis that is determined utilizing a naïve Bayes or Bayesian framework and the individual's clinical data. Process 100 can begin with obtaining (101) an initial set of clinical data. In several embodiments, clinical data is any data that provides an indication of prognosis, especially when utilized within a naïve Bayes or Bayesian framework. In many embodiments, an initial set of clinical data that is gathered. An initial set of clinical data is set of clinical to be utilized within a naïve Bayes or Bayesian framework to determine an initial clinical assessment. Typically, initial set of clinical data is obtained prior to administration of a course of treatment. For various cancers, an initial set of clinical data can include (but is not limited to) international prognostic index (IPI), initial circulating tumor DNA (ctDNA), molecular cell of origin, initial medical imaging (e.g., X-ray MRI, CT, PET scans), choice of first-line therapy, clinical stage, tumor grade, and gene biomarker status. It is to be understood that the clinical data to be collected is tailored to the disorder that is being assessed and the naïve Bayes or Bayesian framework to be utilized. For any particular disorder, in accordance with some embodiments, clinical data to be utilized is data that has been determined to provide a prognostic assessment on that disorder. For example, in some embodiments, the disorder is DLBCL and an initial set of clinical data includes (but not limited to) IPI, molecular cell of origin, ctDNA quantity (prior to treatment), and PET scans for imaging. In some embodiments, the disorder is CLL and an initial set of clinical data includes (but is not limited to) first line therapy and IPI. In some embodiments, the disorder is BRCA and an initial set of clinical data includes (but is not limited to) clinical stage, tumor grade, and status of estrogen receptor (ER) and human epidermal growth factor receptor 2 (HER2) mutational status. In some embodiments, the disorder is NSCLC, and the initial set of clinical data includes (but is not limited to) gross tumor volume, KEAP1 mutational status, and histology. In some embodiments, the disorder is a chronic medical condition such as diabetes mellitus, and an initial set of clinical data includes (but is not limited to) age, type of diabetes, fasting blood glucose, hemoglobin A1C, and comorbidities. In some embodiments, the disorder is an acute medical condition such as sepsis, and an initial set of clinical data includes (but is not limited to) blood pressure, heart rate, temperature, respiratory rate, oxygenation status, and blood counts.

Process 100 also determines (103) an initial clinical assessment utilizing a naïve Bayes or Bayesian framework and the initial set of clinical data. In numerous embodiments, a clinical assessment is a likelihood of an outcome based on the initial sets of clinical data. In some embodiments, a clinical assessment indicates a likelihood to survive a disorder. In some embodiments, a clinical assessment indicates a likelihood of a particular event to occur. In some embodiments, a clinical assessment indicates a likelihood of a reoccurrence of a particular event (e.g., relapse of the disorder). In some embodiments, the disorder is cancer and a clinical assessment indicates event free survival (EFS). In some embodiments, the disorder is cancer and a clinical assessment indicates overall survival (OS). In some embodiments, the disorder is cancer and a clinical assessment indicates progression free survival (PFS). In some embodiments, the disorder is cancer and a clinical assessment indicates distant-relapse free survival (DRFS).

In some embodiments, a naïve Bayes model that determines a prognosis at particular endpoint (e.g., 24 months post initial treatment) is utilized. In some embodiments, a Bayesian framework is a Bayesian model that incorporates Cox proportional hazard to determine a dynamic prognosis over an extended period of time.

In numerous embodiments, a naïve Bayes or Bayesian framework is developed using data of a cohort of patients, wherein the data of each patient includes the status of at least one set of clinical data and the patient's outcome. Accordingly, in various embodiments, data is collected from each patient of a cohort, each patient having a diagnosis of a particular disorder, an assessment for a number of sets of clinical data, and the outcome over some period of time. Based on the cohort data, a naïve Bayes or Bayesian framework can be built that establishes a baseline model of prognostic outcome and how a particular status of each set of clinical data alters the prognostic outcome. In some embodiments, cohort clinical data and outcome data is derived from a cohort study that has already been performed (e.g., manuscript publication or clinical trial results). Specific details on how to develop a naïve Bayes or Bayesian framework including discussion of specific examples can be found herein within the Exemplary Embodiments.

Utilizing a naïve Bayes or Bayesian framework, the initial set of clinical data of an individual is incorporated into the framework to yield a personal clinical assessment. In a number of embodiments, the initial set of clinical data and the naïve Bayes or Bayesian framework determines an initial clinical assessment.

A number of embodiments also utilize a naïve Bayes or Bayesian framework to determine (105) benefit of a particular therapy. A naïve Bayes or Bayesian framework can be further developed utilizing data of courses of treatment from a cohort of patients. In various embodiments, cohort patient data includes for a number of individuals each individual's clinical assessment, the particular course of treatment each individual received, and each individual's outcome of that treatment. Accordingly, a naïve Bayes or Bayesian framework can determine whether a particular course of treatment given for a particular set of initial clinical assessment data results in a more favorable, less favorable, or equivalently favorable result.

To determine benefit of a therapy, clinical assessment data of an individual is incorporated into a naïve Bayes or Bayesian framework that considers a particular course of treatment to yield a prognosis for that individual if it were to receive that particular course of treatment. In some embodiments, multiple treatment Bayesian frameworks for a particular disorder are developed and utilized, each framework has been developed to determine the benefit of a particular course of treatment. It should be further understood that multiple therapies can be combined into a single naïve Bayes or Bayesian framework, as appropriate.

Based on the initial clinical assessment as determined utilizing a naïve Bayes or Bayesian framework, a course treatment begins (107) for the disorder in accordance with a number of embodiments. A number of treatments can be performed, which would be specific to disorder being treated and the prognosis indicated. In several embodiments, an initial clinical assessment indicates that a course of treatment is to be administered, which is often the standard of care. In some embodiments, when initial clinical assessment is unfavorable, a more aggressive treatment is to be utilized than the typical standard of care. In some embodiments, when an initial clinical assessment is favorable, a less aggressive treatment is to be utilized than the standard of care, such as periodic clinical monitoring over time. Further discussion of treatments for particular disorders is provided within the section entitled Applications and Therapies.

During the course of treatment, a subsequent set of clinical data can be obtained (109). As stated previously, in a number of embodiments, clinical data is any data that provides an indication of prognosis. In many embodiments, a subsequent set of clinical data includes data that assesses the current state of disorder. Subsequent sets of clinical data can include (but is not limited to) early molecular response (EMR), major molecular response (MMR), interim medical imaging (e.g., X-ray MRI, CT, PET scans), interim minimal residual disease (MRD), final MRD, ctDNA concentration (i.e., molecular residual disease) and response to treatment. It is to be understood that the subsequent set of clinical data to be collected will be particular to the disorder that is being assessed. For any particular disorder, in accordance with some embodiments, a subsequent set of clinical data to be utilized is data that has been determined to have a significant prognostic ability on that disorder. For example, in some in some embodiments, the disorder is DLBCL and subsequent clinical data includes (but not limited to) ctDNA quantity to determine EMR (during early treatment), ctDNA quantity to determine MMR (during later treatment), and PET scans for interim imaging. In some embodiments, the disorder is CLL and subsequent clinical data includes (but is not limited to interim MRD and final MRD. In some embodiments, the disorder is BRCA and subsequent clinical data includes (but is not limited to) pathological response to therapy. In some embodiments, the disorder is NSCLC and subsequent clinical data includes (but is not limited to) ctDNA molecular residual disease at mid-treatment or post-treatment time-points. In some embodiments, the disorder is a chronic medical condition such as diabetes mellitus, and subsequent clinical data includes (but is not limited to) serial fasting blood glucose and hemoglobin A1C measurements. In some embodiments, the disorder is an acute medical condition such as sepsis, and subsequent clinical data includes (but is not limited to) blood culture results, serial blood pressure measurements, heart rate, temperature, respiratory rate, oxygenation status, and blood counts.

It is to be understood that subsequent clinical data can be obtained anytime during or after therapy, but is to be a point in time subsequent to the initial clinical data. In embodiments in which the treatment is directed at a cancer, a subsequent clinical data can be obtained during or after a surgical procedure, during or after a cycle of chemotherapy treatment, during or after a neoadjuvant treatment, during or after induction therapy, and during or after immunotherapy. In some embodiments, subsequent clinical data is obtained during a period of surveillance after treatment, especially when reoccurrence of the disorder is possible.

Utilizing obtained subsequent clinical data, a naïve Bayes or Bayesian framework can be utilized to update (111) a clinical assessment. In a number of embodiments, a clinical assessment is updated by incorporating the subsequent clinical data in a naïve Bayes or Bayesian framework that was utilized to make an initial prognosis. When a Bayesian framework considers initial and subsequent clinical data, an updated prognosis considers all the risk data acquired thus far, which should result in a robust prognosis considering many relevant factors. In addition, when initial and subsequent clinical data are utilized in a naïve Bayes or Bayesian framework, recency bias is mitigated (i.e., bias towards the last risk assessment). In some embodiments, the benefit of therapy based on an updated prognosis is also determined (113), in a similar manner as described is step 105. Accordingly, a naïve Bayes or Bayesian framework can determine whether a particular course of treatment given for particular sets of initial and subsequent clinical assessment data results in a more favorable, less favorable, or equivalently favorable result. In several embodiments, a subsequent clinical indicates the course of treatment is to be maintained. In some embodiments, a subsequent clinical assessment is less favorable than the initial clinical assessment and the subsequent course of treatment is more aggressive treatment than the initial course of treatment. In some embodiments, a subsequent clinical assessment is more favorable than the initial clinical assessment and the subsequent course of treatment is a less aggressive treatment than the initial course of treatment.

In addition, further subsequent sets of clinical data can be repeatedly obtained throughout the course and beyond treatment. Accordingly, a clinical assessment can be repeatedly updated with each subsequent set of clinical data collected. As such, a prognosis can (and will likely) change with each additional acquisition of clinical data. Based on an updated clinical assessment, a course of treatment can be altered. In some embodiments, when a prognosis worsens, a course of treatment is updated to be more aggressive and/or prolonged than the initial course of treatment. And conversely, in some embodiments, when a prognosis improves, a course of treatment is updated to be less aggressive and/or shortened than the initial course of treatment. In a number of embodiments, a therapy is either added or removed, based on the updated clinical assessment. And in some embodiments, a course of treatment is reinitiated, especially in scenarios when a prognosis worsens during a period of surveillance after a course of treatment has concluded.

While specific examples of processes for determining and updating a personalized clinical assessment utilizing a naïve Bayes or Bayesian framework are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for determining and updating a personalized clinical assessment utilizing a naïve Bayes or Bayesian framework appropriate to the requirements of a given application can be utilized in accordance with various embodiments of the invention.

### Applications and Treatments

Various embodiments are directed to treatments based on determining a prognosis utilizing a naïve Bayes or Bayesian framework. As described herein, sets of clinical data can be obtained before, during, and/or after a treatment to obtain a clinical assessment, which can be updated repeatedly as each subsequent set of clinical data is acquired and entered into a Bayesian framework. Based on a prognosis, treatments may be performed on a patient.

### Cancer Diagnostics and Treatments

A number of embodiments are directed towards treating an individual for a neoplasm and/or cancer. Accordingly, an individual's risk factor data can be collected and entered into a Bayesian framework to determine the individual's prognosis in relationship to that neoplasm and/or cancer. As described herein, risk factor data can be collected before, during, or after a treatment and a prognosis can be updated with each collection of risk factor data. This methodology allows an individual's prognosis to be dynamic and thus allows a treatment plan to be updated based on the prognosis.

In accordance with various embodiments, numerous types of neoplasms and cancers can be assessed and treated, including (but not limited to) acute lymphoblastic leukemia (ALL), acute myeloid leukemia (AML), anal cancer, astrocytomas, basal cell carcinoma, bile duct cancer, bladder cancer, breast cancer, breast adenocarcinoma (BRCA), cervical cancer, chronic lymphocytic leukemia (CLL) chronic myelogenous leukemia (CML), chronic myeloproliferative neoplasms, colorectal cancer, endometrial cancer, ependymoma, esophageal cancer, diffuse large B-cell lymphoma (DLBCL), esthesioneuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors.

In accordance with many embodiments, once a prognosis is indicated, a number of treatments can be performed, including (but not limited to) surgery, chemotherapy, radiation therapy, immunotherapy, targeted therapy, hormone therapy, stem cell transplant, and blood transfusion. In some embodiments, an anti-cancer and/or chemotherapeutic agent is administered, including (but not limited to) alkylating agents, platinum agents, taxanes, vinca agents, anti-estrogen drugs, aromatase inhibitors, ovarian suppression agents, endocrine/hormonal agents, bisphosphonate therapy agents and targeted biological therapy agents. Medications include (but are not limited to) cyclophosphamide, fluorouracil (or 5-fluorouracil or 5-FU), methotrexate, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolomide, topotecan, vincristine, vinblastine, eribulin, mutamycin, capecitabine, capecitabine, anastrozole, exemestane, letrozole, leuprolide, abarelix, buserelin, goserelin, megestrol acetate, risedronate, pamidronate, ibandronate, alendronate, zoledronate, tykerb, daunorubicin, doxorubicin, epirubicin, idarubicin, valrubicin mitoxantrone, bevacizumab, cetuximab, ipilimumab, ado-trastuzumab emtansine, afatinib, aldesleukin, alectinib, alemtuzumab, atezolizumab, avelumab, axitinib, belimumab, belinostat, bevacizumab, blinatumomab, bortezomib, bosutinib, brentuximab vedotin, brigatinib, cabozantinib, canakinumab, carfilzomib, ceritinib, cetuximab, cobimetinib, crizotinib, dabrafenib, daratumumab, dasatinib, denosumab, dinutuximab, durvalumab, elotuzumab, enasidenib, erlotinib, everolimus, gefitinib, ibritumomab tiuxetan, ibrutinib, idelalisib, imatinib, ipilimumab, ixazomib, lapatinib, lenvatinib, midostaurin, necitumumab, neratinib, nilotinib, niraparib, nivolumab, obinutuzumab, ofatumumab, olaparib, olaratumab, osimertinib, palbociclib, panitumumab, panobinostat, pembrolizumab, pertuzumab, ponatinib, ramucirumab, regorafenib, ribociclib, rituximab, romidepsin, rucaparib, ruxolitinib, siltuximab, sipuleucel-T, sonidegib, sorafenib, temsirolimus, tocilizumab, tofacitinib, tositumomab, trametinib, trastuzumab, vandetanib, vemurafenib, venetoclax, vismodegib, vorinostat, and ziv-aflibercept. In accordance with various embodiments, an individual may be treated, by a single medication or a combination of medications described herein. A common treatment combination is cyclophosphamide, methotrexate, and 5-fluorouracil (CMF).

Dosing and therapeutic regimes can be administered appropriate to the neoplasm to be treated, as understood by those skilled in the art. For example, 5-FU can be administered intravenously at dosages between 25 mg/m² and 1000 mg/m².

In some embodiments, medications are administered in a therapeutically effective amount as part of a course of treatment. As used in this context, to “treat” means to ameliorate at least one symptom of the disorder to be treated or to provide a beneficial physiological effect. For example, one such amelioration of a symptom could be reduction of tumor size and/or risk of relapse.

A therapeutically effective amount can be an amount sufficient to prevent reduce, ameliorate or eliminate the symptoms of colorectal cancer. In some embodiments, a therapeutically effective amount is an amount sufficient to reduce the growth and/or metastasis of a colorectal cancer.

Many embodiments are directed to diagnostic or companion diagnostic scans performed during cancer treatment of an individual. When performing diagnostic scans during treatment, the ability of agent to treat the neoplastic growth can be monitored. Most anti-cancer therapeutic agents result in death and necrosis of neoplastic cells, which should release higher amounts nucleic acids from these cells into the samples being tested. Accordingly, the level of neoplastic nucleic acids (e.g., ctDNA) can be monitored over time, as the level should increase during treatments and begin to decrease as the number of neoplastic cells are decreased.

Various embodiments are also directed to diagnostic scans performed after treatment of an individual to detect residual disease and/or recurrence of neoplastic growth. If a diagnostic scan indicates residual and/or recurrence of neoplastic growth, further diagnostic tests and/or treatments may be performed as described herein. If the neoplastic growth and/or individual is susceptible to recurrence, diagnostic scans can be performed frequently to monitor any potential relapse.

### EXEMPLARY EMBODIMENTS

The embodiments of the invention will be better understood with the several examples provided within. Many exemplary results of processes that provide continuous risk assessment utilizing clinical data are described. Validation results are also provided.

### Example 1: Dynamic Risk Profiling Using Serial Tumor Biomarkers for Personalized Outcome Prediction

### Summary

Accurate prediction of long-term outcomes remains a challenge in the care of cancer patients. Due to the difficulty of serial tumor sampling, previous prediction tools have focused on pretreatment factors. However, emerging non-invasive diagnostics have increased opportunities for serial tumor assessments. Described within this example is the Continuous Individualized Risk Index (CIRI), an exemplary method to dynamically determine outcome probabilities for individual patients utilizing risk-predictors acquired over time. CIRI provides a real-time probability by integrating risk-assessments throughout a patient's course. Applying CIRI to patients with diffuse large B-cell lymphoma, improved outcome prediction was demonstrated, as compared to conventional risk-models. CIRI's broader utility was further demonstrated in analogous models of chronic lymphocytic leukemia and breast adenocarcinoma, and proof-of-concept analysis demonstrated how CIRI could be used to develop predictive biomarkers for therapy selection. Based on the examples described herein, dynamic risk-assessment facilitates personalized medicine and enables innovative therapeutic paradigms.

### INTRODUCTION

Biological and clinical heterogeneity between patients remain inherent barriers to improving cancer outcomes. Such variation contributes to dramatically different outcomes for patients nominally sharing the same disease. Over the past five decades, significant efforts have been made in unraveling this heterogeneity between patients through refined classifications that capture physical, anatomical, radiographic, histological, and molecular features of tumors. For example, by identifying patients with similar clinical phenotypes, anatomic staging systems have enabled more uniform treatment practices, leading to improvement in outcomes for patients. Similarly, molecular dissection of differing biological features has resulted in the identification of targetable subtypes in diverse tumors, including HER2 amplifications in breast cancer and EGFR mutations in lung cancers, also leading to improved patient outcomes.

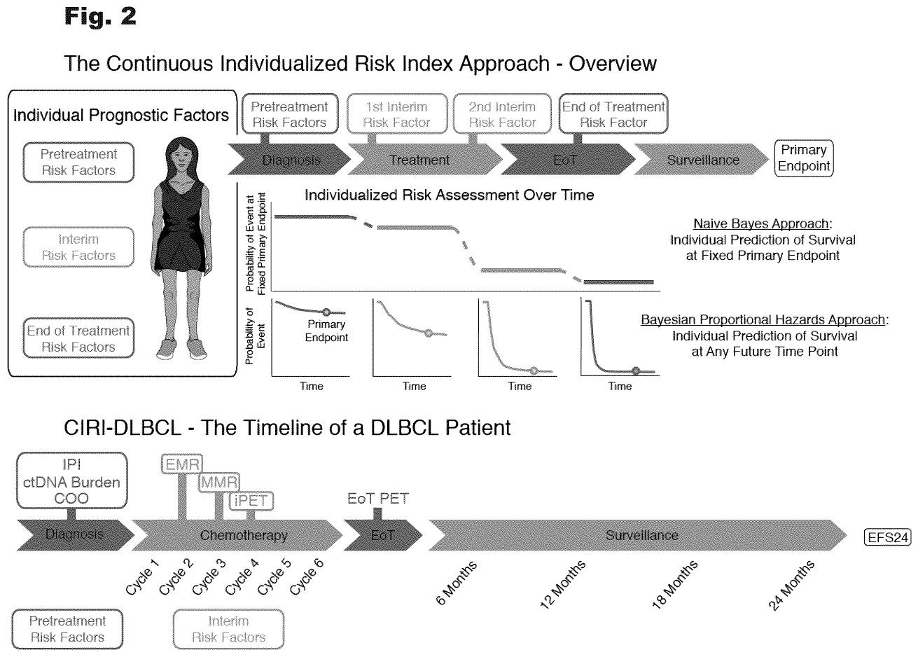

Despite such advances, however, significant heterogeneity remains in most cancer subtypes. For example, within the most common hematologic cancer, diffuse large-B cell lymphoma (DLBCL), systemic therapy cures the majority of patients; however, a significant minority will succumb to disease. Several prognostic tools to stratify DLBCL patients into risk groups are currently employed, utilizing clinical (International prognostic index, IPI), molecular (Cell-of-origin, COO) (A. A. Alizadeh, et al., Nature 403, 503-511 (2000); and A. Rosenwald, et al., The New England journal of medicine 346, 1937-1947 (2005); the disclosures of which are each incorporated herein by reference), or radiographic (Interim positron emission tomography, iPET) features (FIG. 2) (V. Safar et al., Journal of clinical oncology: official journal of the American Society of Clinical Oncology 30, 184-190 (2012); and C. A. Thompson, et al., Journal of clinical oncology: official journal of the American Society of Clinical Oncology 32, 3506-3512 (2014); the disclosures of which are each incorporated herein by reference). However, prior studies utilizing these methods to select patients for intensified therapy have failed to improve overall survival.

Previous risk stratification tools have largely focused on pretreatment factors due to the difficulty of obtaining serial tumor biopsies. Despite this, an early response to systemic therapy is a strong prognostic factor in many cancers, including hematologic and solid tumors. Innovative tools such as liquid biopsies are rapidly emerging and allow serial assessments of tumor burden with relative ease. For example, a recent reported demonstrated high prognostic performance of circulating tumor DNA (ctDNA) after one or two cycles of systemic therapy (Early and Major Molecular Response; EMR/MMR) for predicting outcomes in patients with DLBCL (D. M. Kurtz, et al., Journal of clinical oncology: official journal of the American Society of Clinical Oncology, JCO2018785246 (2018), the disclosure of which is incorporated herein by reference). Similarly, other response-based surrogates of outcome have proven strongly predictive in other cancers. Circulating minimal residual disease (MRD) following therapy of chronic lymphocytic leukemia (CLL) is strongly associated with outcomes after diverse therapies. Separately, pathological complete responses (pCR) to neoadjuvant therapy have been suggested as predictive of ultimate outcomes in several cancer types, including invasive ductal carcinomas of the breast. These tools, however, fundamentally separate patients into ‘risk categories’ based on an assessment at a fixed time-point either before or during therapy, which can lead to misclassification of certain patients. For example, over 50% of DLBCL patients in the ‘high-risk’ IPI category will ultimately be cured with frontline therapy (M. Ziepert, et al., Journal of clinical oncology: official journal of the American Society of Clinical Oncology 28, 2373-2380 (2010); the disclosure of which is incorporated herein by reference). To overcome these issues, this example describes the CIRI framework to accurately measure a given patient's risk throughout the disease course—rather than solely identifying at-risk populations of patients prior to treatment—to better resolve clinical heterogeneity and provide superior outcome predictions.

The CIRI framework is a dynamic risk model for integrating diverse outcome predictors into a single quantitative risk estimate for individual patients throughout their disease. As described herein, CIRI has proven utility for dynamic outcome prediction in multiple diseases, including the most common lymphoma subtype (DLBCL), the most common leukemia (CLL), and the most common cancer in women (breast cancer). These cancers have diverse, established outcome predictors, including biomarkers assessed after therapy by noninvasive or invasive means as outlined above. CIRI was compared to proportional hazard modeling, the traditional tool for modeling survival with multiple predictors. It was demonstrated that by integrating prior knowledge of the utility of each biomarker, CIRI outperforms proportional hazard modeling, with proportional hazard models demonstrating similar performance only when large amounts of training data are available. In addition, a proof-of-concept analysis was performed to show that CIRI not only improves accuracy of prognostic models, but also identifies subsets of patients who benefit from specific therapies.

### Results

### Development of a Dynamic Model for Personalized Disease Risk in DLBCL

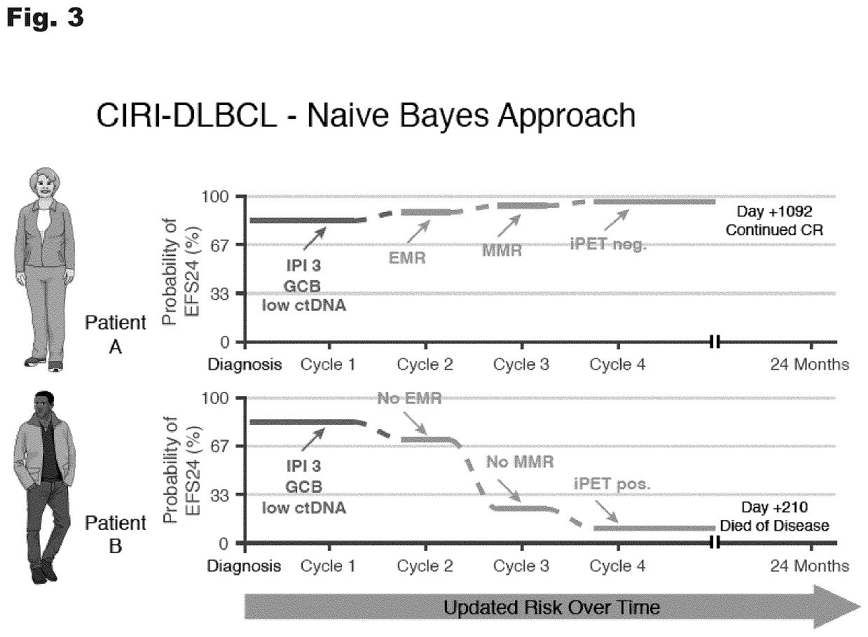

Predictions are made in the context of time and are refined with serially collected, longitudinal data (FIG. 2). For example, in patients with DLBCL, not all risk predictors are available prior to therapy. Therefore, risk-predictions are updated dynamically as additional information becomes available, such as measuring molecular response by ctDNA or interim imaging studies (FIG. 3).

To build a model, ideally case-level medical data would be abundant, however, large archives of case-level medical data are generally lacking. Even less common are complete datasets that include all predictors of interest. For example, few case-level data sources exist that capture both established DLBCL risk factors (such as the IPI, cell of origin, and interim PET) and novel predictors such as ctDNA. To overcome the data deficiency, CIRI was initially developed using a naïve Bayes approach, allowing leverage of group-level prior knowledge on the performance of established tools for risk stratification, which is commonly reported in the literature. This approach also allows serial integration of predictors over time, as described above.

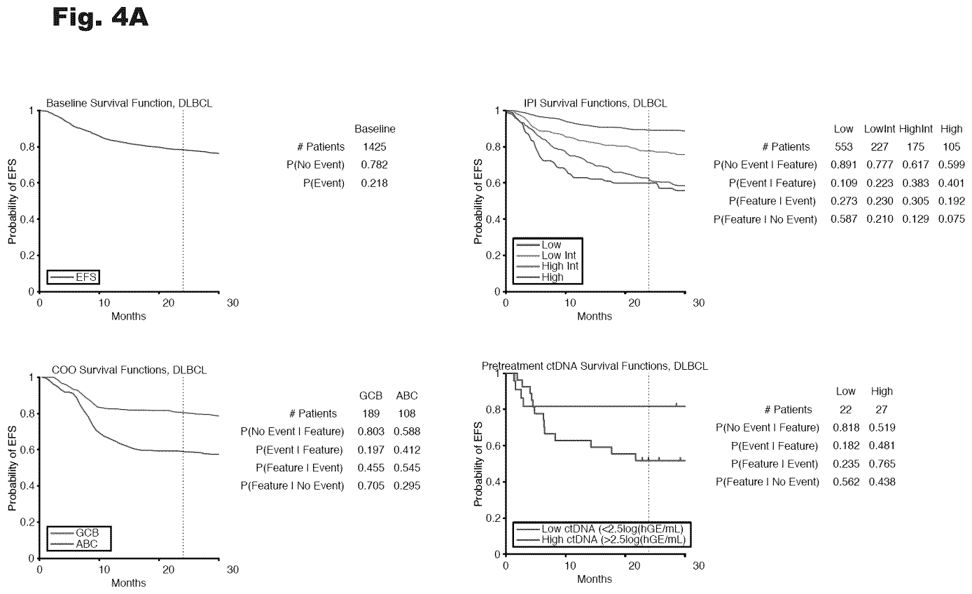

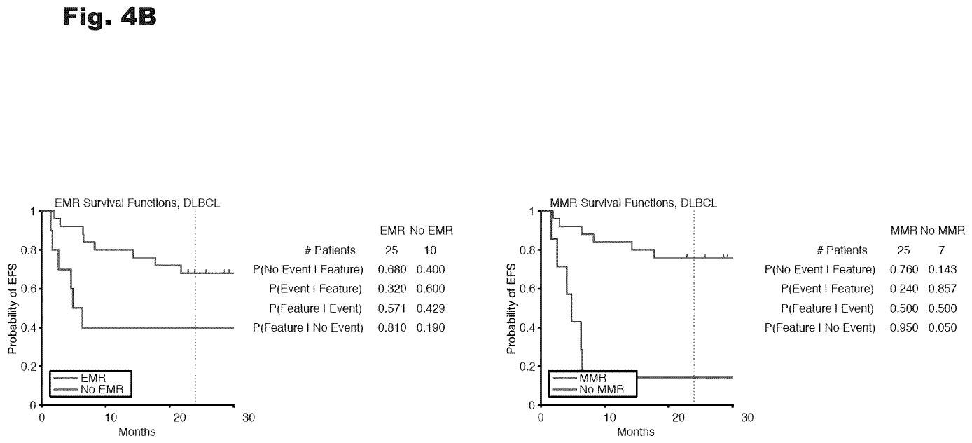

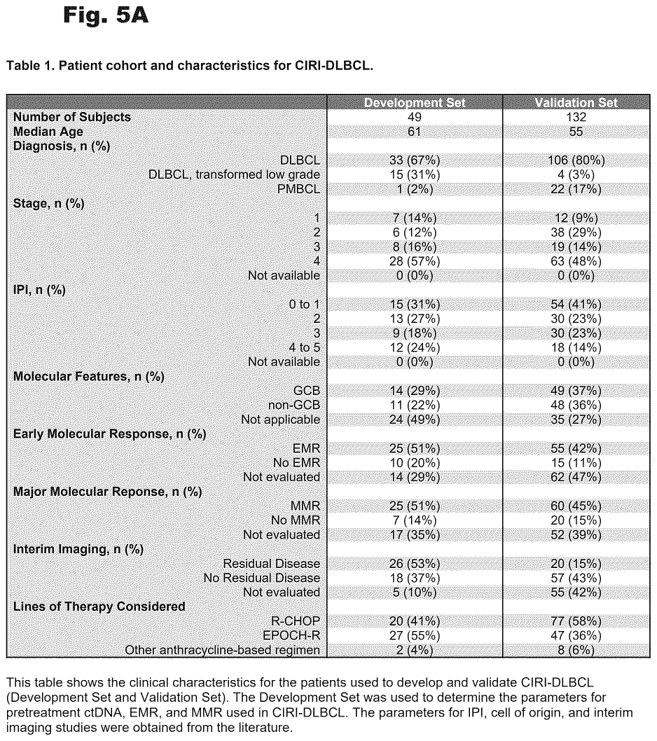

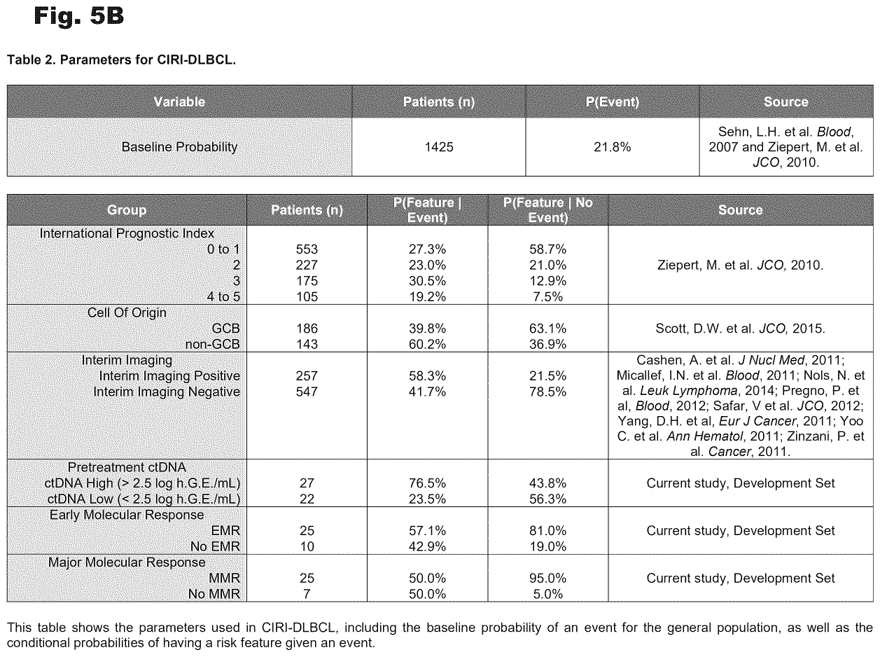

CIRI-DLBCL considers a total of six complementary risk predictors, including three established risk-factors (IPI (L. H. Sehn, et al., Blood 109, 1857-1861 (2007); and M. Ziepert, et al., (20100) cited supra; the disclosures of which are each incorporated herein by reference, molecular cell of origin (D. W. Scott, et al., Journal of clinical oncology: official journal of the American Society of Clinical Oncology 33, 2848-2856 (2015), the disclosure of which is incorporated herein by reference), and interim imaging (A. F. Cashen, Journal of nuclear medicine: official publication, Society of Nuclear Medicine 52, 386-392; I. N. Micallef, et al., Blood 118, 4053-4061 (2011); N. Nols, et al., Leukemia & lymphoma 55, 773-780 (2014); P. Pregno, et al., Blood 119, 2066-2073 (2012); V. Safer, et al., (2012), cited supra; D. H. Yang, et al., European journal of cancer 47, 1312-1318 (2011); C. Yoo, et al., Annals of hematology 90, 797-802 (2011); and P. L. Zinzani, et al., Cancer 117, 1010-1018 (2011); the disclosures of which are each incorporated herein by reference), as well as three ctDNA risk-factors (pretreatment ctDNA levels, EMR, and MMR) (D. M. Kurtz, et al, (2018), cited supra). The CIRI-DLBCL model was assessed at its ability to predict event-free survival at 24 months (EFS24), a clinically relevant milestone and standard endpoint in this disease (FIG. 2). The performance for established risk-factors was determined from a total of 2558 patients in 11 previously published studies to serve as prior information for the model (FIGS. 4A & 4B). No prior knowledge is available for ctDNA levels; thus, the performance of ctDNA was determined in a development set of patients receiving frontline immunochemotherapy (FIG. 5A). The conditional probabilities for individual predictors are also provided in Table 2 (FIG. 5B).

A graphical schema for CIRI depicting its performance for two exemplar patients with similar baseline characteristics is shown in FIG. 3. When a new diagnosis of DLBCL is made, the IPI is typically assessed, providing an initial risk estimate. Additional risk factors, including cell-of-origin and pretreatment ctDNA, can further refine the pretreatment risk estimate. As therapy is introduced, further risk factors, including ctDNA measurements (i.e., EMR, MMR) and interim imaging (i.e., PET/CT) can be obtained and integrated, updating CIRI personalized risk estimates over time.

### CIRI Outcome Predictions Improve on the IPI and Molecular Response in DLBCL

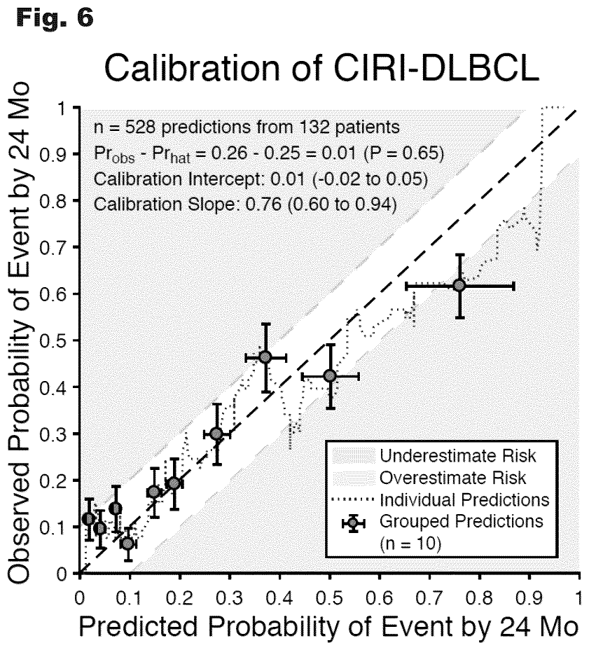

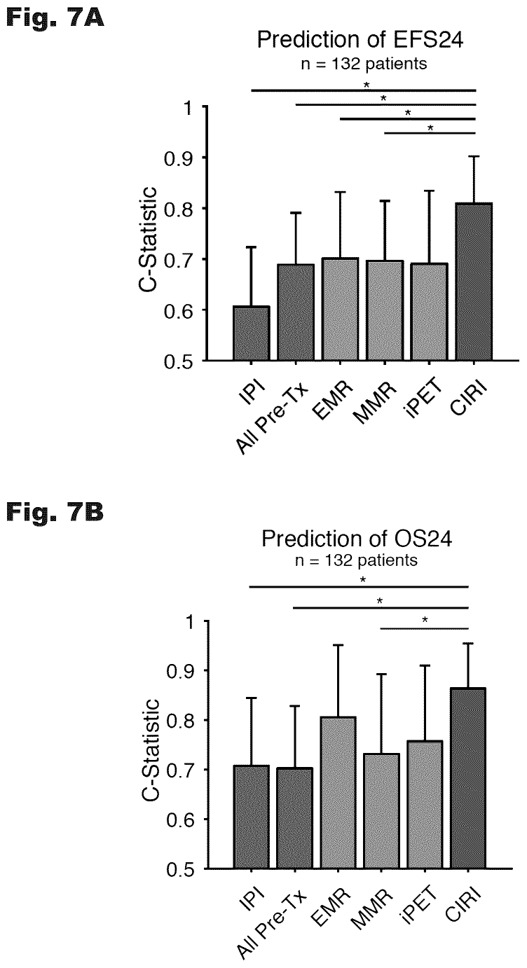

The performance of CIRI-DLBCL was assessed in an independent validation cohort of 132 patients with available ctDNA data; the clinical characteristics of these patients are provided in Table 1 (FIG. 5A). The predictions for both quantitative accuracy (i.e., ‘model calibration’) as well as ability to accurately identify outcomes (i.e., ‘discrimination’) were evaluated. Model calibration was assessed by comparing predicted and observed risks of the entire cohort (‘calibration-in-the-large’) as well as across subgroups of patients with similar risk profiles (via calibration plot and regression. Across the cohort, predictions made by CIRI-DLBCL were calibrated with clinical outcomes—the average predicted risk of event by 24 months was not different from the observed risk (25% vs 26% risk of event, or a 1% overestimate of risk [95% CI-3% to 4%]). Subgroups of patients with similar risk profiles also displayed adequate calibration to observed outcomes (n=528 predictions from 132 patients; FIG. 6). Importantly, prediction of EFS24 by CIRI significantly improved on the IPI when compared by C-statistic (FIG. 7, 0.81 vs 0.61; P<0.001), with similar improvements over EMR, MMR, and interim PET as individual predictors (C-statistic 0.70, 0.70, 0.69; P=0.022, 0.006, and 0.07 respectively). Moreover, while this initial approach was designed to predict EFS at 24 months, stratification of risk by CIRI-DLBCL also significantly improved on prediction of OS at 24 months compared to the IPI (FIG. 7B; 0.86 vs 0.71; P=0.009).

### Extension of CIRI to Longitudinal Survival Data

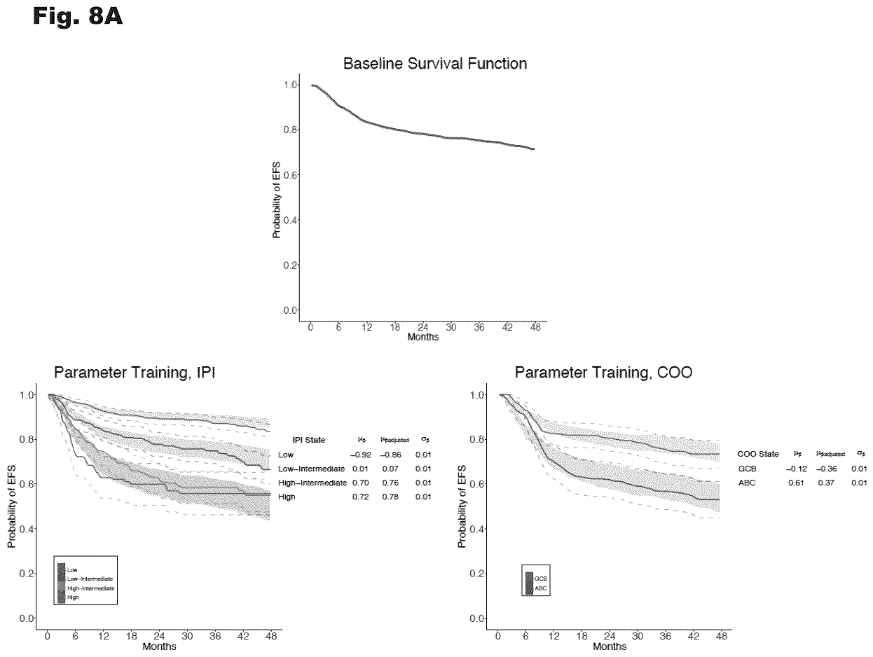

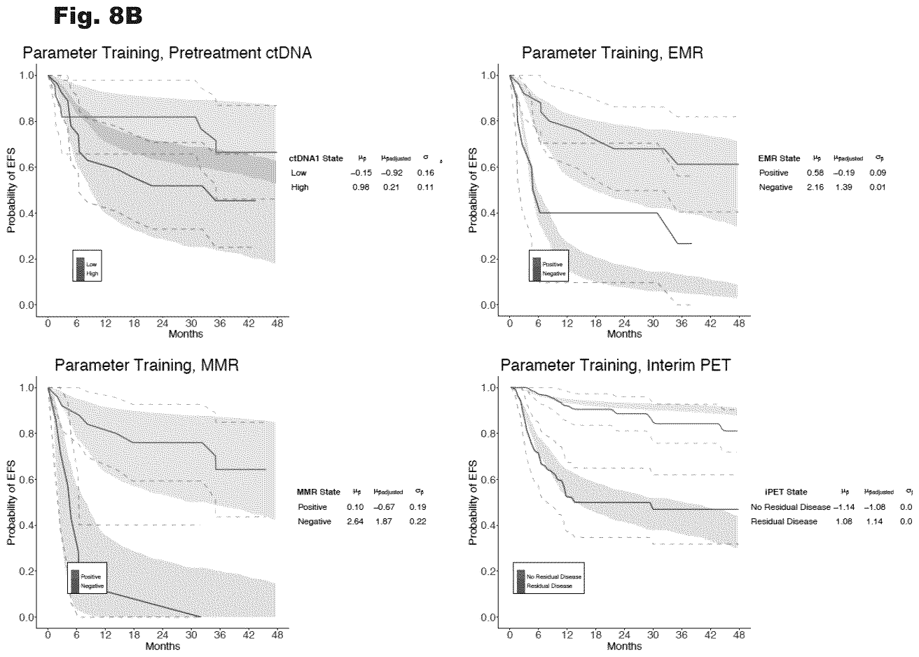

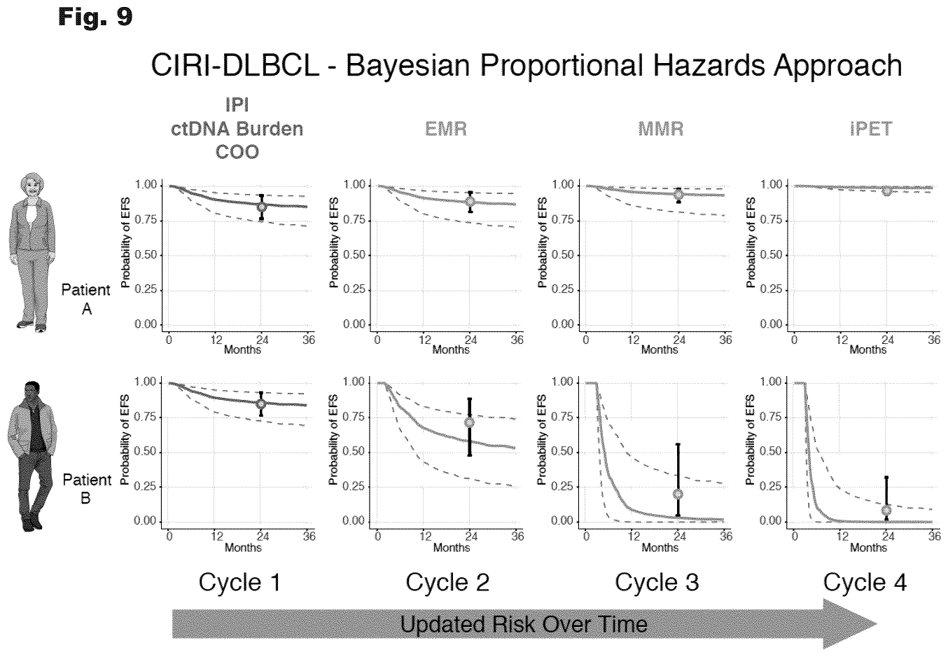

The formulation of CIRI above predicts the probability of an event at a fixed time-point of interest (in the case of DLBCL, EFS at 24 months). However, in many situations, patients and clinicians may not be interested in survival at a fixed point in time, but rather in survival at any time during a course of therapy. CIRI was therefore extended to predict the survival of individual patients at any point over time by utilizing proportional hazard modeling and Bayesian analysis. Similar to the fixed time-point CIRI above, parameters were identified for the model based on previously established literature (FIGS. 8A & 8B). A schema for CIRI predicting survival over time for two patients with similar baseline characteristics is provided (FIG. 9). Similar to FIG. 3, a given patient's probability of survival is updated as more information becomes available. A complete, personalized survival curve is produced, however, rather than a prediction at a fixed point in time. Notably, as this procedure includes predictors obtained after the start of therapy, this process can suffer from ‘guaranteed time bias’. To account for this, the personalized probability of survival beginning from the start of therapy remains at 100%—or ‘guaranteed’—until the time of the most recently obtained risk predictor (see FIG. 9).

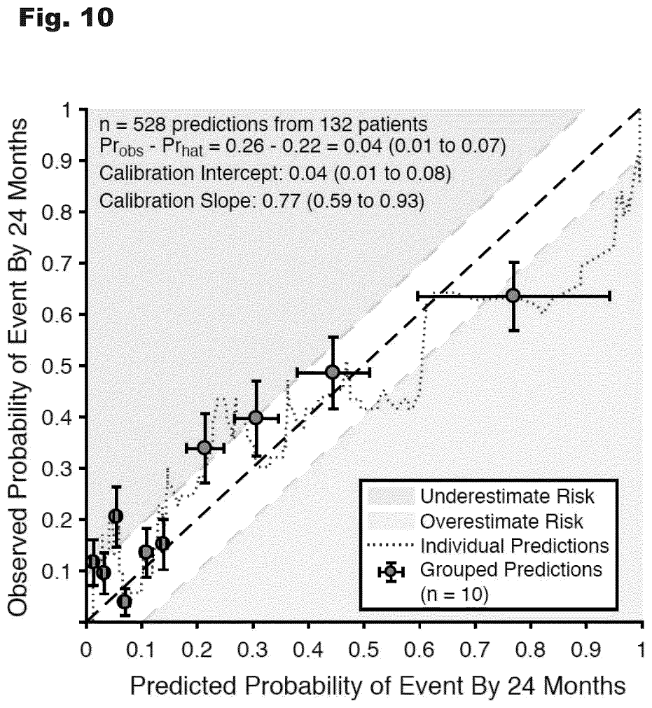

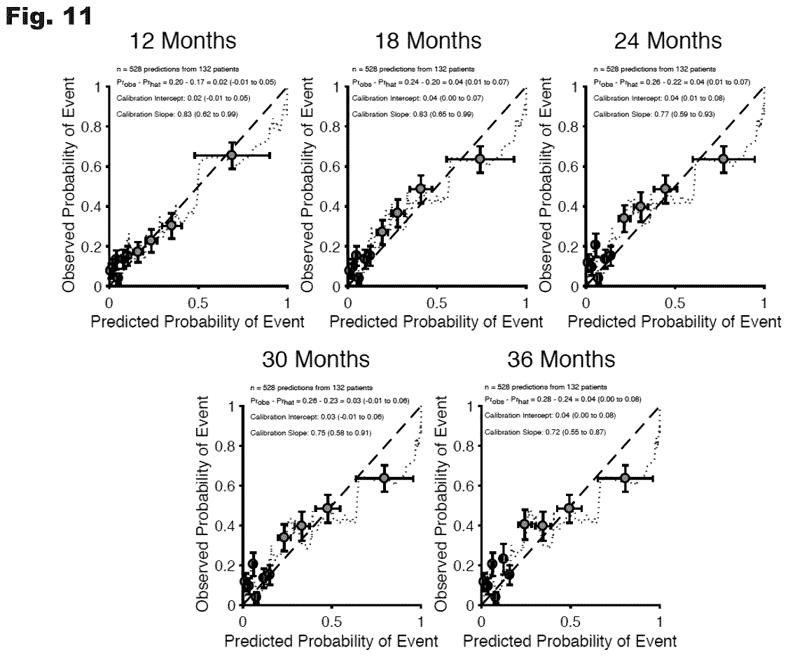

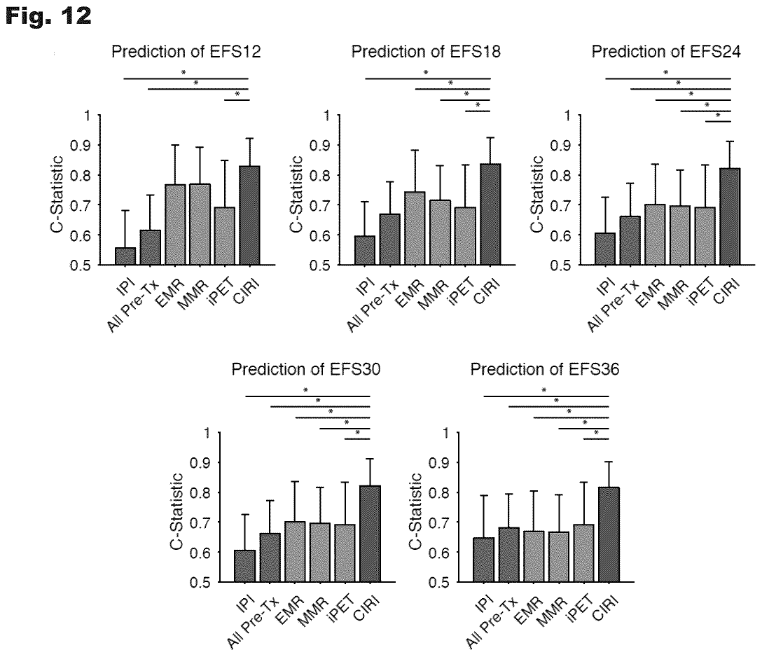

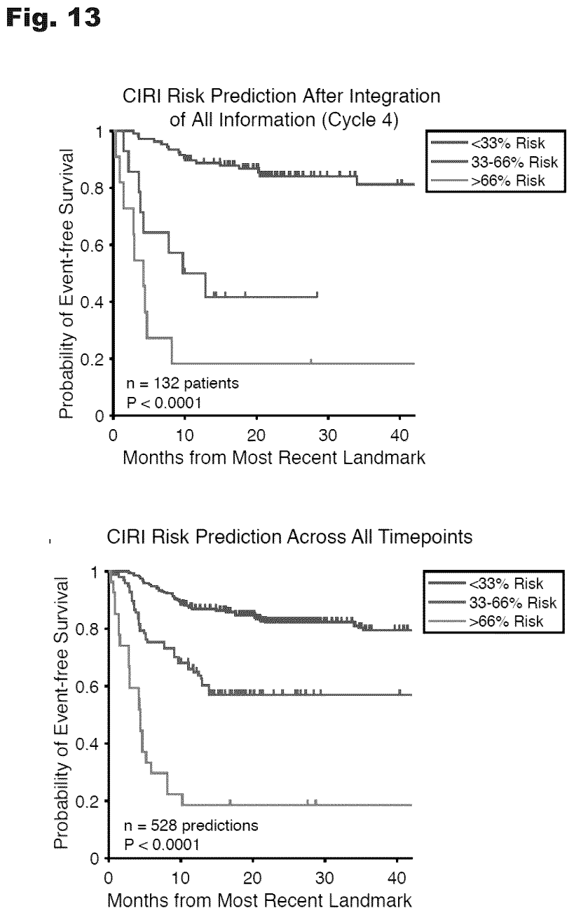

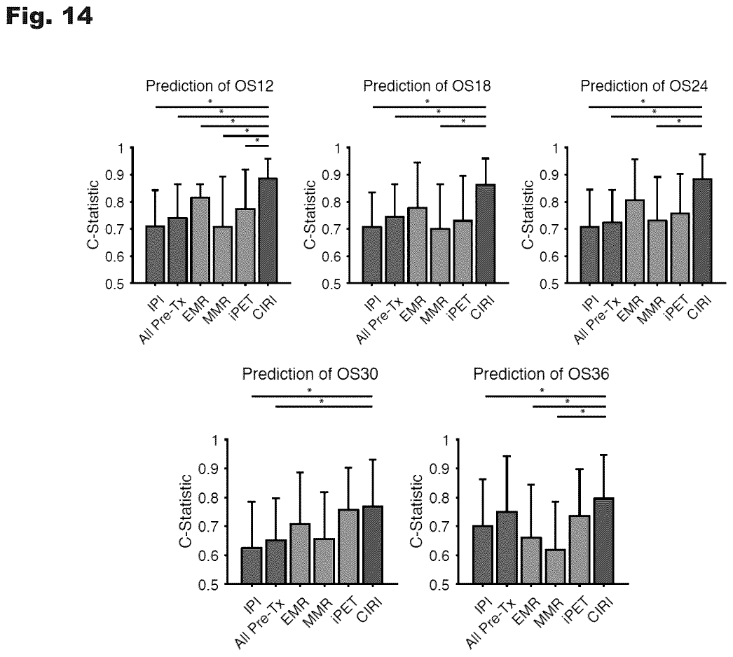

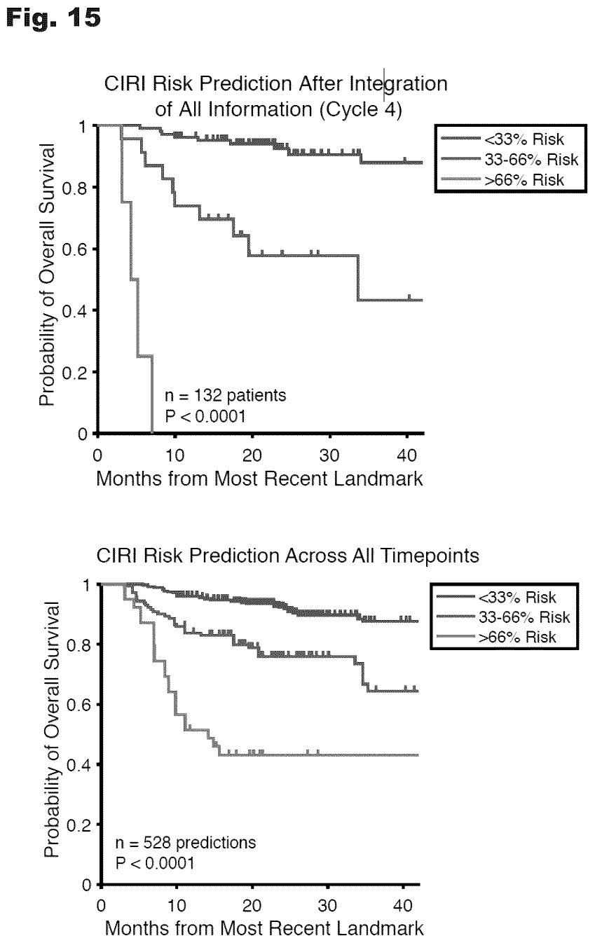

In an independent validation set, prediction of EFS24 demonstrated reasonable calibration-in-the-large, with only a 4% difference (95% C.I.: 1%-7%) between observed and predicted outcomes (FIG. 10). Moreover, CIRI-DLBCL demonstrated adequate calibration of predictions throughout the disease course, with a <5% difference between observed and predicted outcomes when considering event-free survival from 12 to 36 months (FIG. 11). Importantly, CIRI-DLBCL generally outperformed the IPI, pretreatment risk factors, molecular response, and interim PET for prediction of EFS at multiple time-intervals throughout the disease course, ranging from 12 to 36 months (FIG. 12). Furthermore, while CIRI-DLBCL provides a quantitative risk estimate for each patient at each time-point, these predictions can also be separated into risk groups. When considering strata with <33%, 33%-66%, and >66% predicted risk of adverse event by 24 months- or low, medium, and high-risk-CIRI significantly stratified patients when considering either the final prediction after 3 cycles of immunochemotherapy or all risk-predictions in aggregate (FIG. 13). Additionally, a similar CIRI-DLBCL model was constructed for prediction of OS. Again, CIRI-DLBCL improved on prediction of OS at multiple time-intervals throughout the disease course as compared to the IPI and other individual predictors (FIGS. 14 and 15).

### Extension of CIRI to Chronic Lymphocytic Leukemia

To further extend dynamic risk modeling and CIRI, its utility was explored in Chronic Lymphocytic Leukemia (CLL), an indolent malignancy and the most common leukemia in adults. Although CLL and DLBCL are both lymphoid malignancies, a distinct set of risk factors is used in each disease. In CLL, these include clinical and cytogenetic risk indices such as the CLL-IPI (The International CLL-IPI Working Group, The Lancet Oncology 17, 779-790 (2016), the disclosure of which is incorporated herein by reference) and minimal residual disease (MRD) levels from peripheral blood cells (S. Bottcher, et al., Journal of clinical oncology: official journal of the American Society of Clinical Oncology 30, 980-988 (2012); G. Kovacs, et al., Journal of clinical oncology: official journal of the American Society of Clinical Oncology 34, 3758-3765 (2016); and M. Kwok, et al., Journal of clinical oncology: official journal of the American Society of Clinical Oncology, JCO2018785246 (2018); the disclosures of which are each incorporated herein by reference). Furthermore, as treatment selection is a major prognostic factor in CLL (B. Eichhorst, et al., The Lancet Oncology 17, 928-942 (2016); V. Goede, et al., The New England journal of medicine 370, 1101-1110 (2014); and M. Hallek, et al., Lancet 376, 1164-1174 (2010); the disclosures of which are each incorporated herein by reference), the effect of therapy was considered on outcomes in CIRI-CLL.

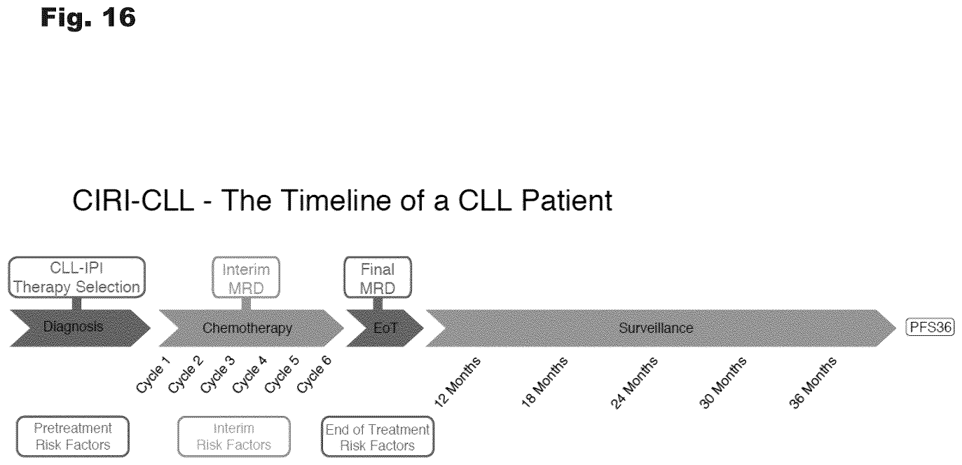

A timeline for a typical patient with CLL is shown in FIG. 16. The prognostic value of each of these features has been previously demonstrated in prior studies. Case-level data from three such studies from the German CLL Study Group—CLL8, CLL10, and CLL11 was reanalyzed. These studies prospectively assessed the effect of therapy for CLL in a large population of patients. Notably, each study also had MRD data available at interim and final restaging assessed by flow cytometry (CLL8 and CLL10) or qPCR (CLL11). 1426 patients were identified from these 3 studies with at least one available MRD assessment after initiation of therapy.

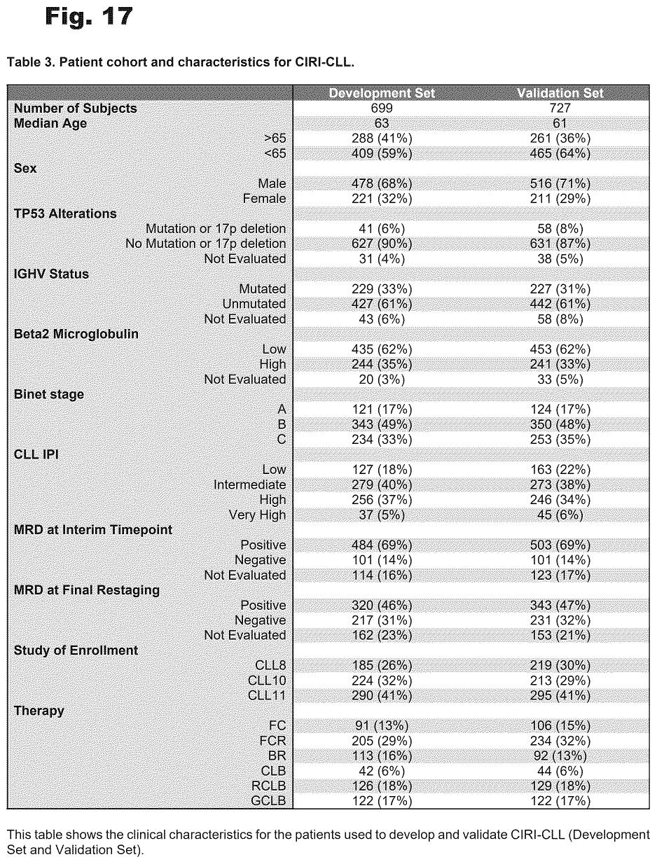

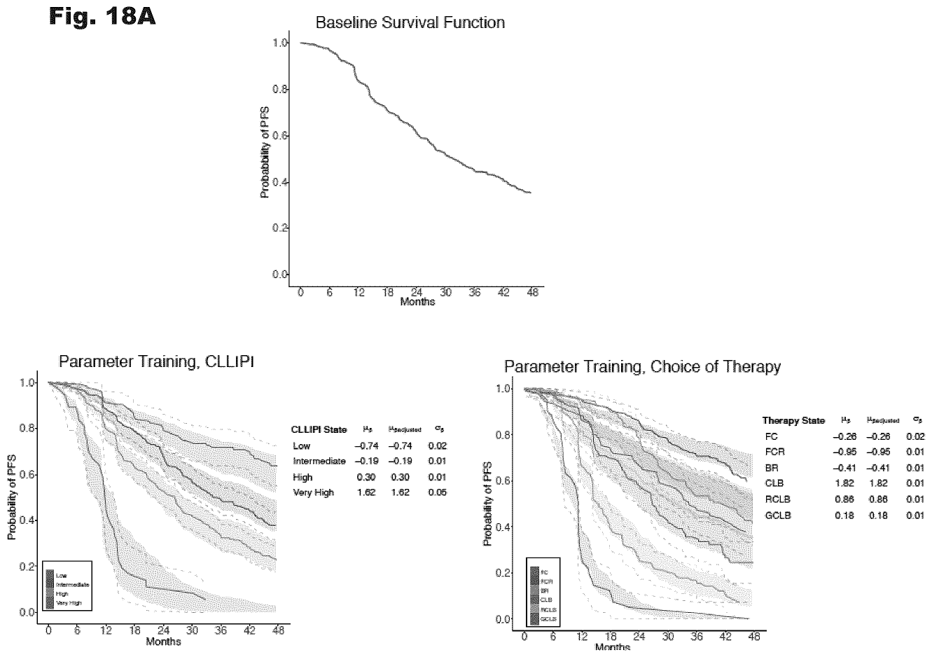

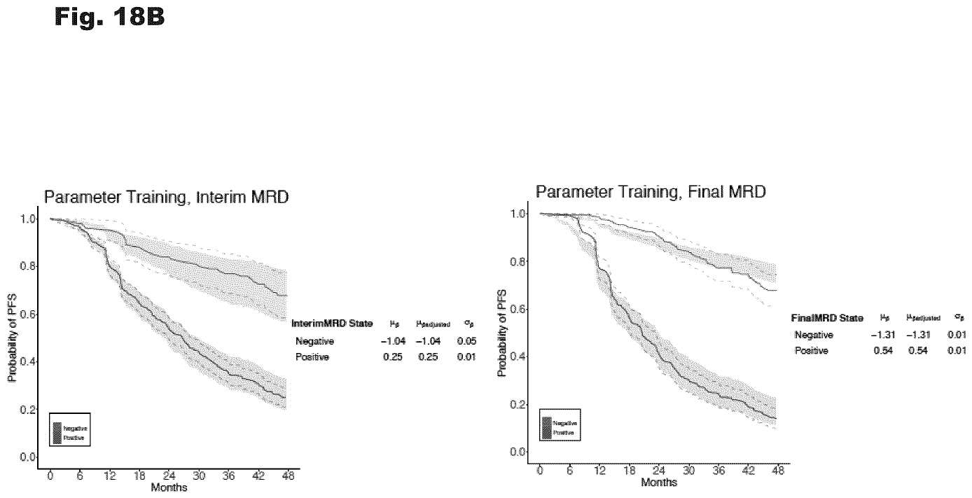

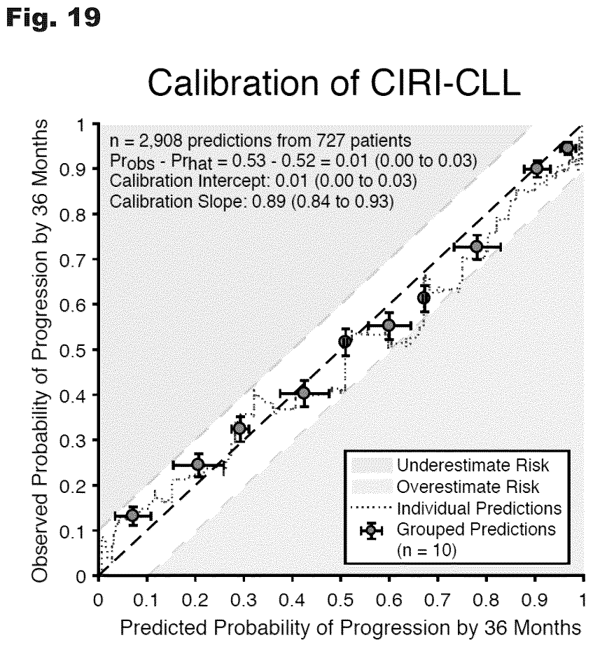

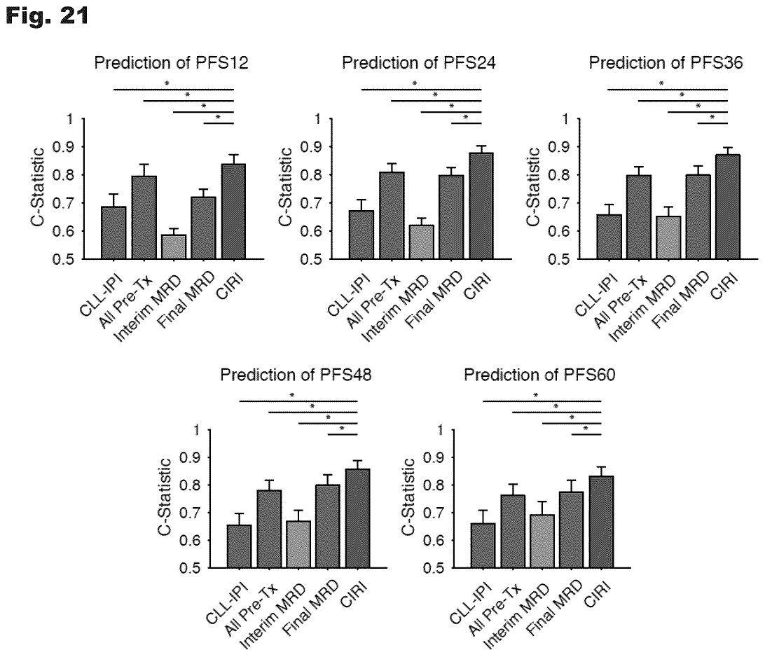

Patients were randomly assigned into development and validation sets; the characteristics of these patients are provided in Table 3 (FIG. 17). Using the development set to serve as the ‘prior knowledge’, the parameters for CIRI-CLL were determined, including the predictive value of CLL-IPI, interim and final MRD measurement, and choice of therapy, using the framework to predict a personalized probability of progression-free survival (PFS) over time (FIGS. 18A & 18B). The performance of CIRI-CLL was then assessed in the independent validation set. Similar to DLBCL, predictions made by CIRI-CLL were adequately calibrated with observed outcomes for predictions of PFS at 12, 24, 36, and 48 months (FIGS. 19 & 20). Specifically, there was <5% difference between observed and predicted outcomes in the validation set at each of these time-points. Prediction of PFS by CIRI-CLL outperformed CLL-IPI and MRD assessment by C-statistic across all time-points considered (FIG. 21).

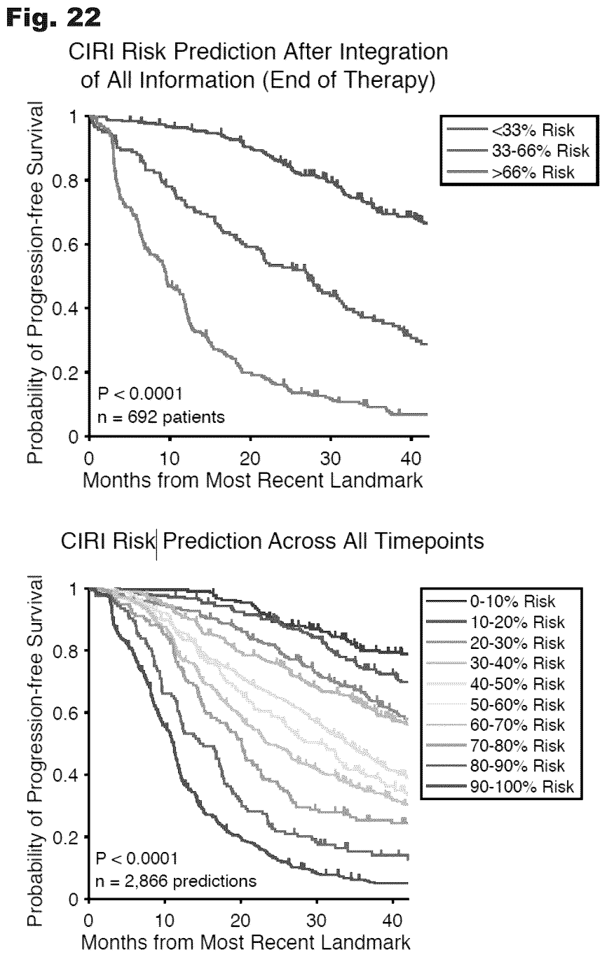

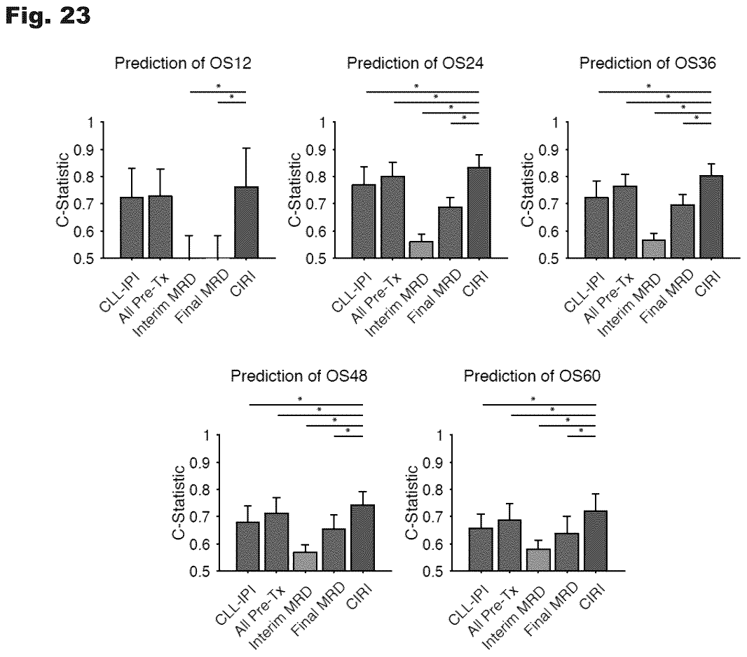

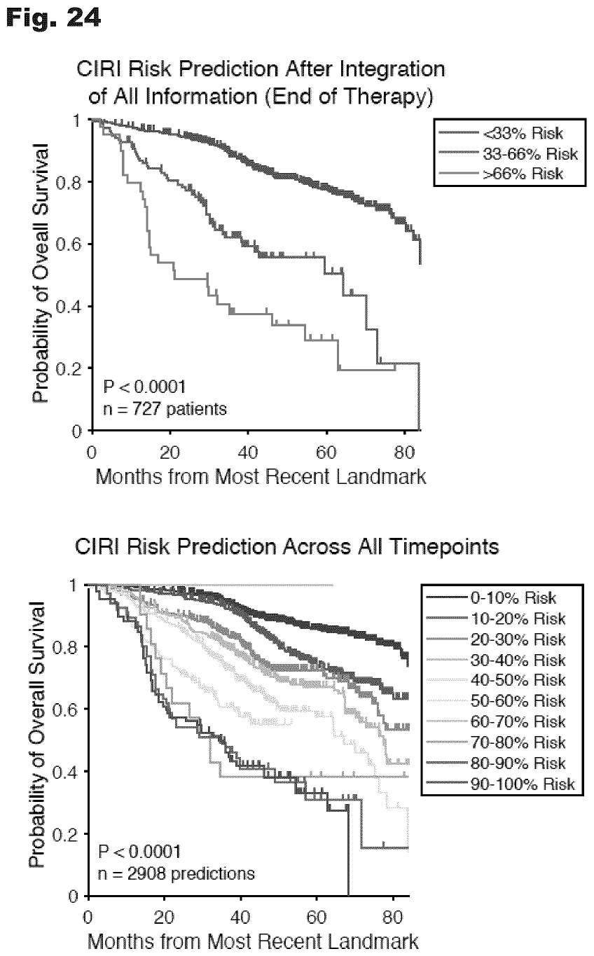

Similar to CIRI-DLBCL, when stratified based on predicted probability of reaching PFS36, CLL patients could be separated into risk strata with defined risk profiles (FIG. 22; P<0.0001). Given the larger number of subjects and predictions, this model provides an opportunity to further assess the ability of CIRI to stratify patients based on predicted risk. Considering all risk-predictions divided into ten groups, CIRI-CLL demonstrated robust and quantitative stratification of patient outcomes (FIG. 22; P<0.0001). A CIRI-CLL model was additionally constructed for prediction of OS. Similar to the model of PFS, stratification of risk by CIRI-CLL significantly improved on prediction of OS compared to the CLL-IPI and MRD assessment alone at all time horizons longer than 12 months, including an improvement in C-statistic from 0.72 to 0.80 for OS36 (P<0.001; FIGS. 23 and 24).

### Extension of CIRI to Neoadjuvant Chemotherapy in Breast Cancer

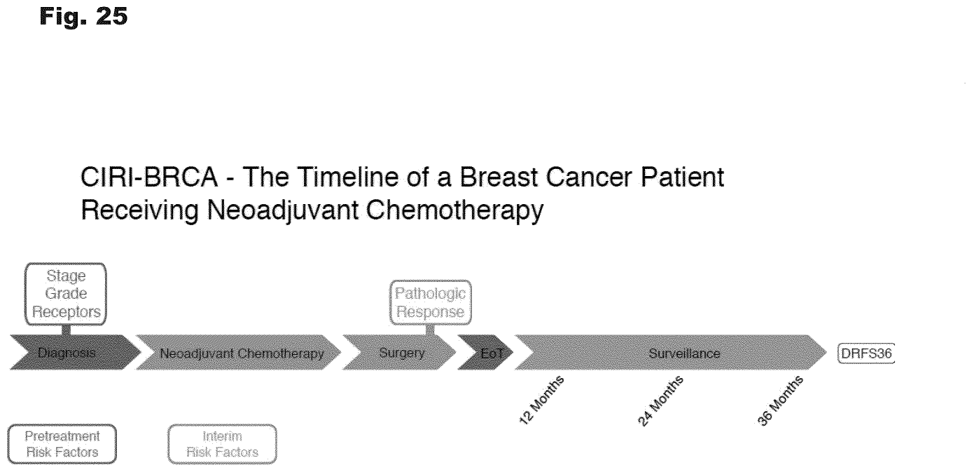

While detection of residual disease from either cell-free DNA or via cell-based approaches lends itself to dynamic risk assessment, this concept is more widely applicable to any longitudinal data that can be quantified during a course of therapy. To exemplify this, a CIRI risk model of localized breast adenocarcinomas (BRCA) treated with a combination of neoadjuvant chemotherapy followed by definitive surgical resection was constructed. A timeline for a typical patient with breast cancer receiving neoadjuvant chemotherapy prior to surgical resection is provided (FIG. 25). In this context, CIRI-BRCA utilizes risk-factors obtained both prior to and during therapy; these indices include clinical stage, tumor grade, estrogen receptor and HER2 status (obtained pretreatment), as well as pathological response to neoadjuvant chemotherapy (i.e., residual cancer burden, assessed after neoadjuvant therapy and resection).

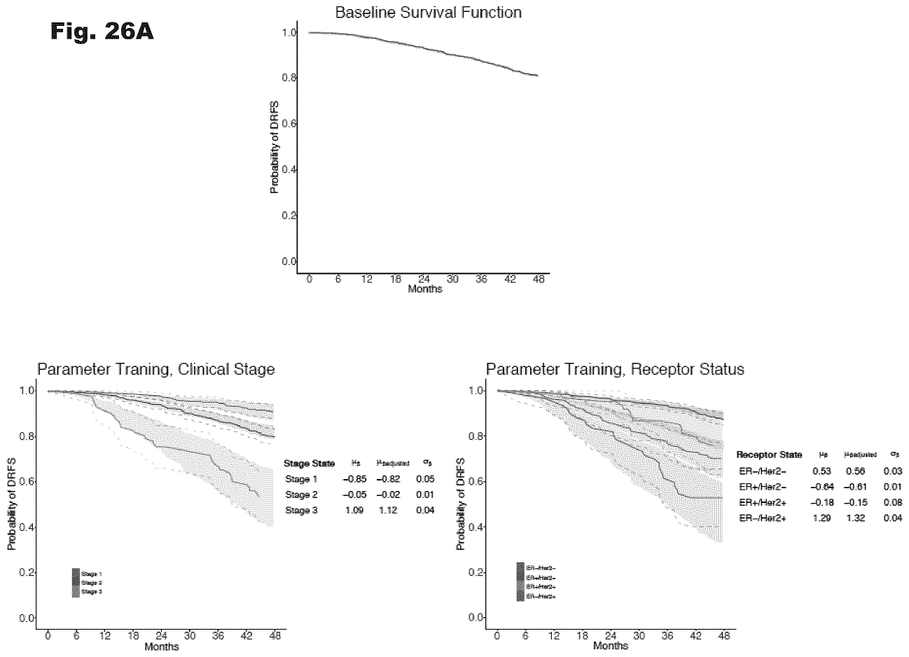

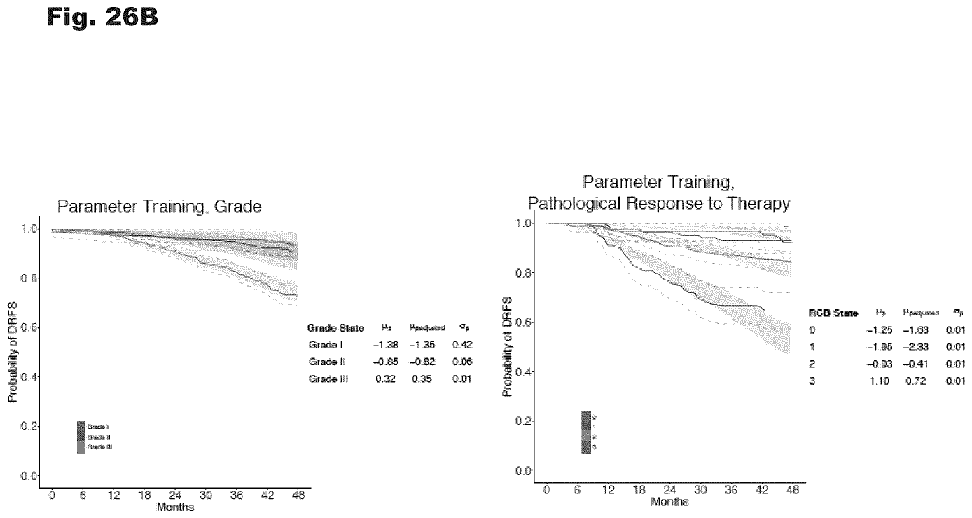

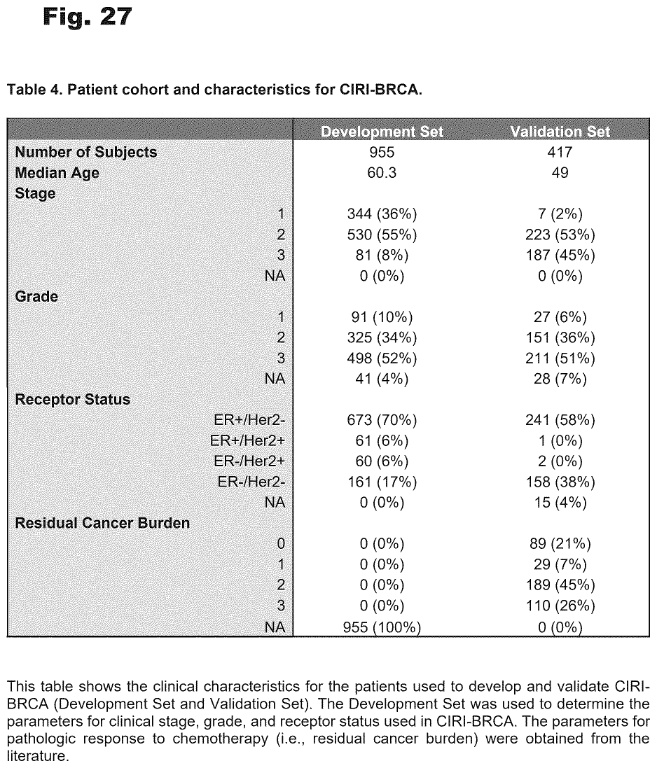

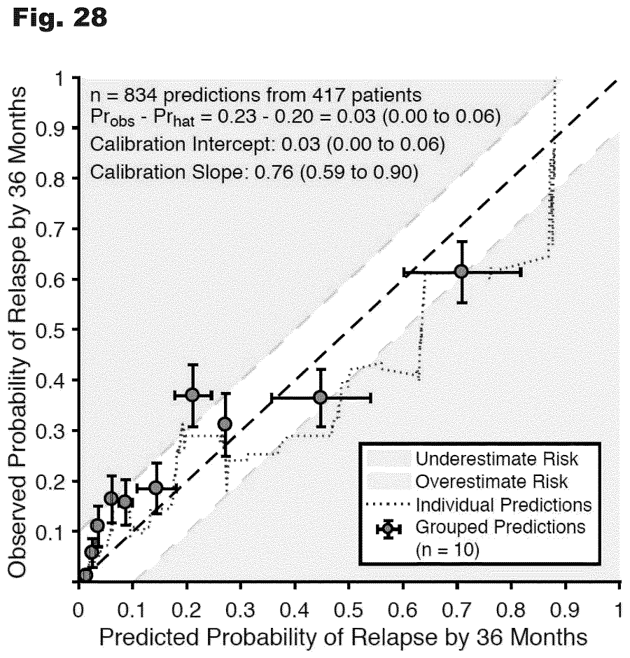

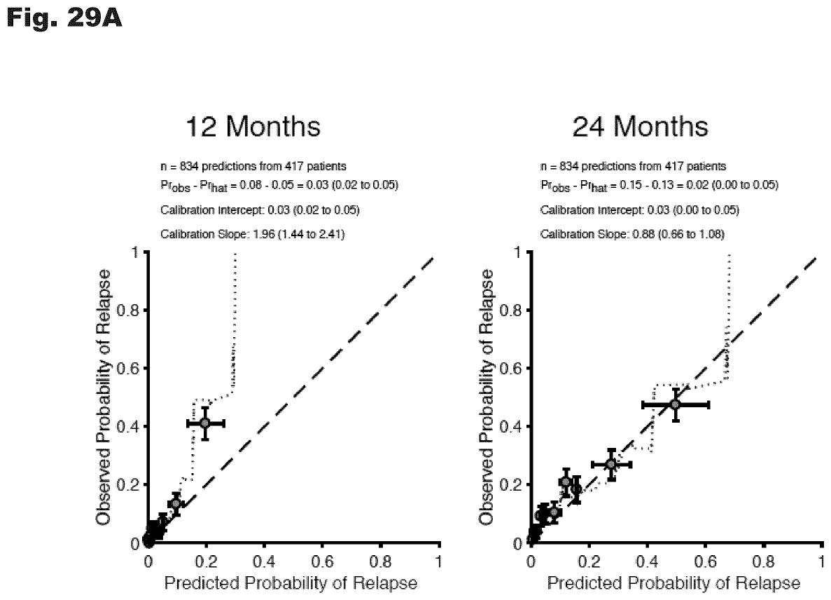

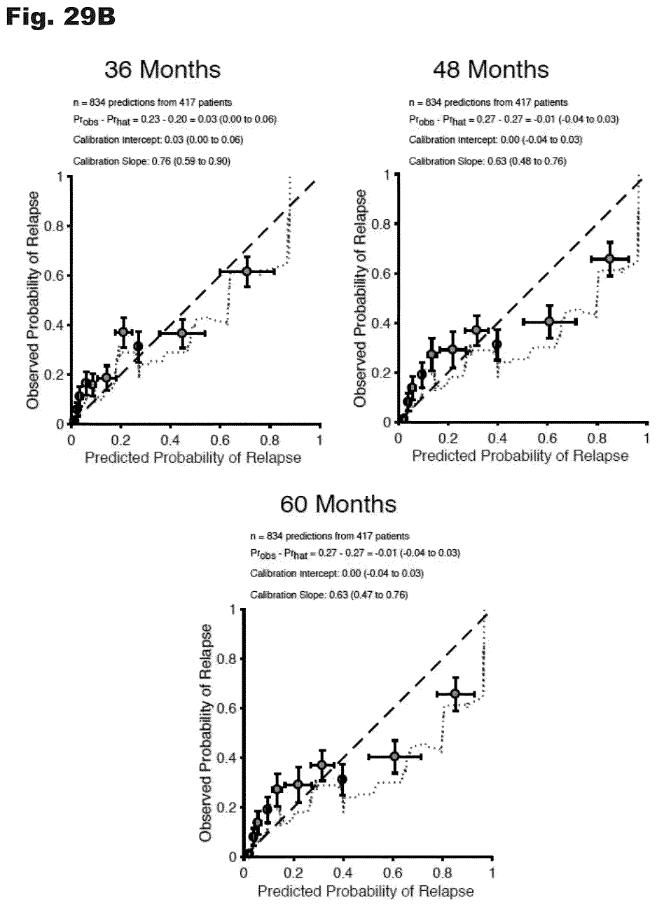

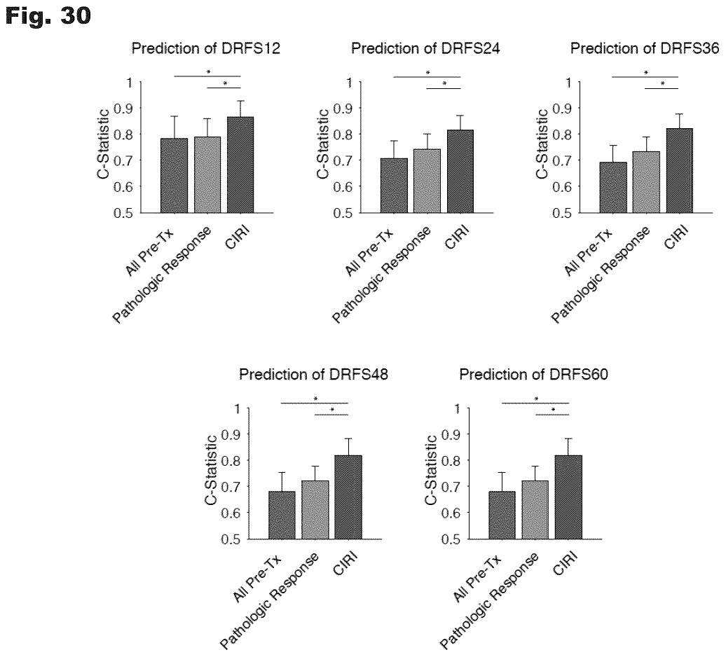

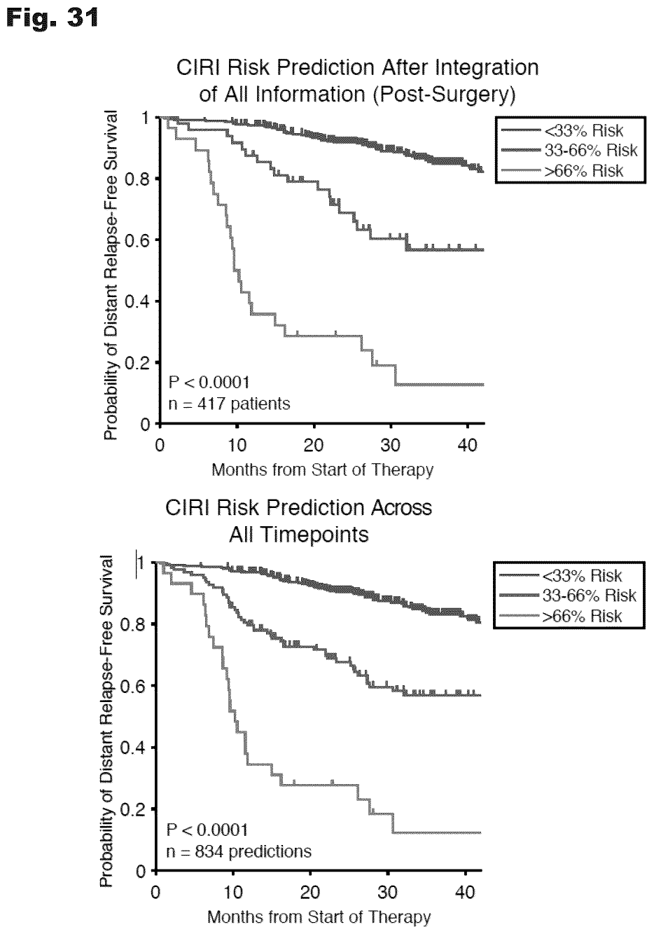

The parameters for CIRI-BRCA for each of these risk factors were established from published literature (C. Curtis, et al., Nature 486, 346-352 (2012); and W. F. Symmans, et al., Journal of clinical oncology: official journal of the American Society of Clinical Oncology 35, 1049-1060 (2017), the disclosures of which are each incorporated herein by reference) (FIGS. 26A & 26B); the performance of the CRIR-BRCA model was then assessed in an independent, publicly available cohort of 417 patients (C. Hatzis, et al., JAMA 305, 1873-1881 (2011), the disclosure of which is incorporated herein by reference). The characteristics of the patients used to develop and validate CIRI-BRCA are shown in Table 4 (FIG. 27). Similar to the DLBCL and CLL models, predictions made by CIRI-BRCA demonstrated acceptable calibration across endpoints from 12 to 60 months (FIGS. 28, 29A, and 29B). Furthermore, predictions made by CIRI-BRCA improved on predictions made using pretreatment factors or pathologic response assessment alone (FIG. 30). CIRI-BRCA significantly stratified patients with similar risk profiles for distant relapse-free survival both at the completion of therapy and throughout the course of treatment (FIG. 31). Overall survival data was not available for this cohort.

### Effect of Correlated Predictors on CIRI

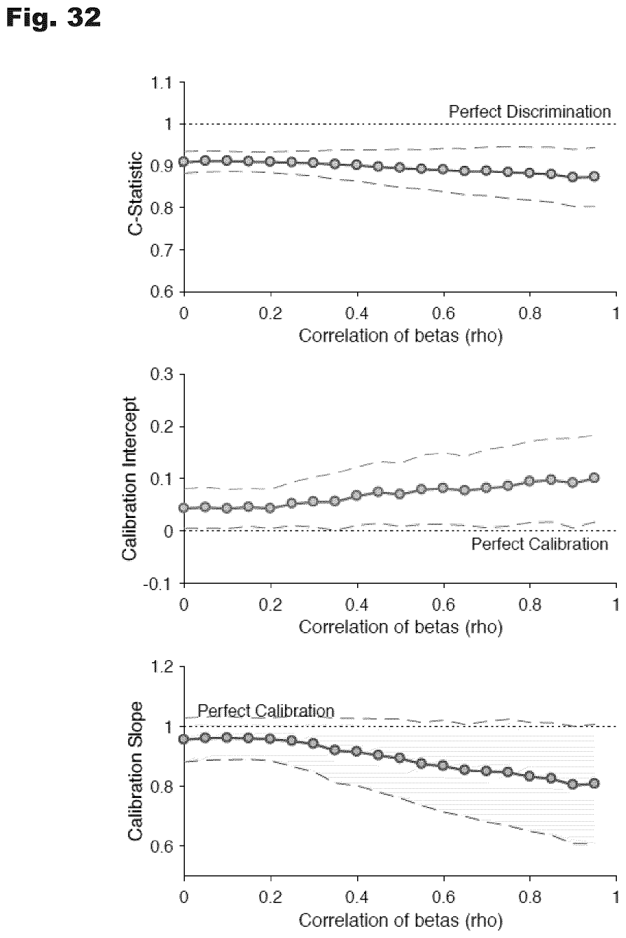

One of the assumptions behind CIRI is the independence of the effects of individual risk predictors in the prior distribution. To test the robustness of CIRI using with a Bayesian proportional hazard approach to correlation of these predictors, datasets containing an increasing amount of correlation between each risk factor were simulated. The effect of increasing correlation was assessed on the performance of CIRI, both for classification of and quantitative calibration with observed outcomes. CIRI remained robust to increasing levels of correlation, with minimal change in classification performance (i.e., C-Statistic) and degradation of model calibration only at high levels of correlation (FIG. 32).

### Comparison of CIRI to Proportional-Hazard Modeling

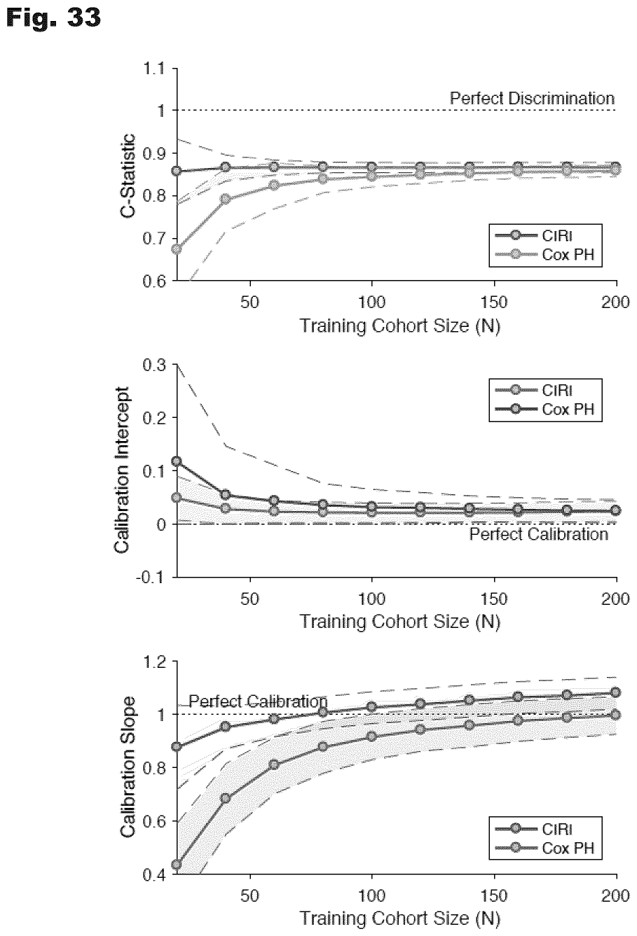

The CIRI framework outlined here has a number of advantages over alternative methods, including the ability to incorporate prior knowledge of the performance of each outcome predictor as established from prior literature or datasets. The performance of CIRI was compared to proportional hazard models that were not informed by prior knowledge in the CLL and breast cancer datasets. As proportional hazard models typically require case level training data, the validation set in each case was utilized to develop and cross-validate the model. Interestingly, when the amount of case-level training data was low (<100 cases), CIRI outperformed standard Cox proportional hazard models, both in terms of identifying clinical outcomes and calibration with quantitative outcomes (FIGS. 33 & 34). However, as the number of cases increased, the predictive performance of proportional hazard modeling converged toward the performance of CIRI for both discrimination of clinical outcomes (i.e., C-Statistic) and model calibration.

Prediction of Therapeutic-Benefit within Specific Disease Subgroups

To utilize CIRI as a predictive biomarker, an ‘induction’ period of therapy is given, followed by an assessment of response to therapy. Based on the combination of this dynamic evaluation and pretreatment risk factors, the best therapy for a specific patient could then be selected. To simulate this potential use-case, a CIRI-CLL model was constructed combining the CLL-IPI and interim MRD and incorporating the choice of initial therapy. This model was applied to the CLL dataset in a 10-fold cross-validation framework to identify patients who preferentially benefit from FCR compared to alternative immuno-chemotherapies after the interim MRD time-point.

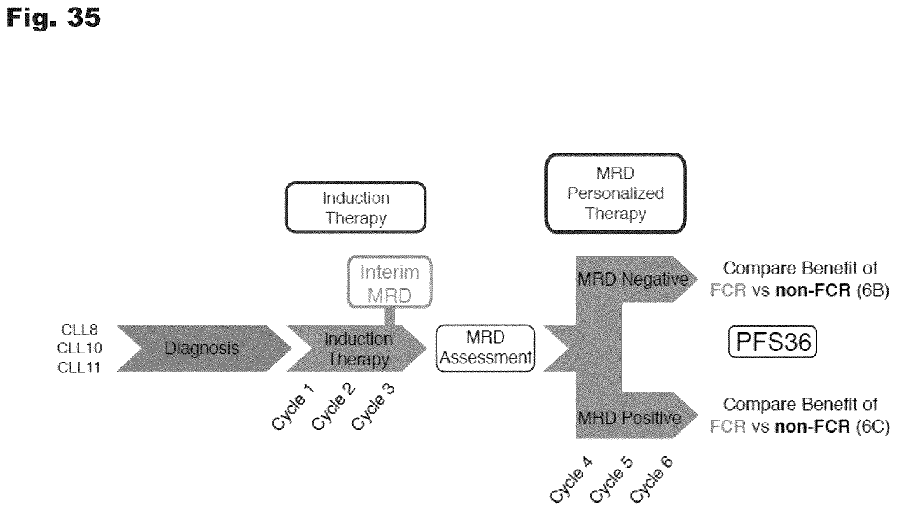

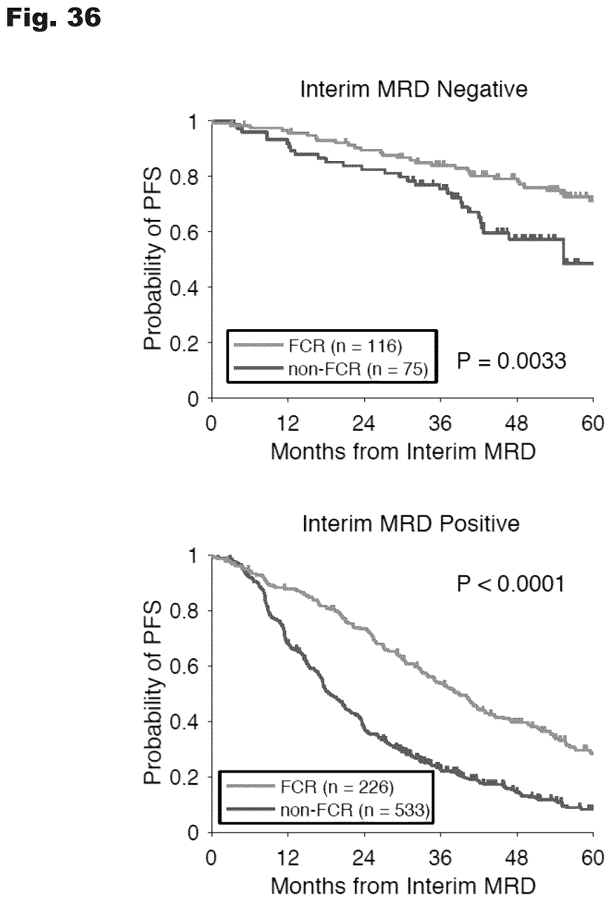

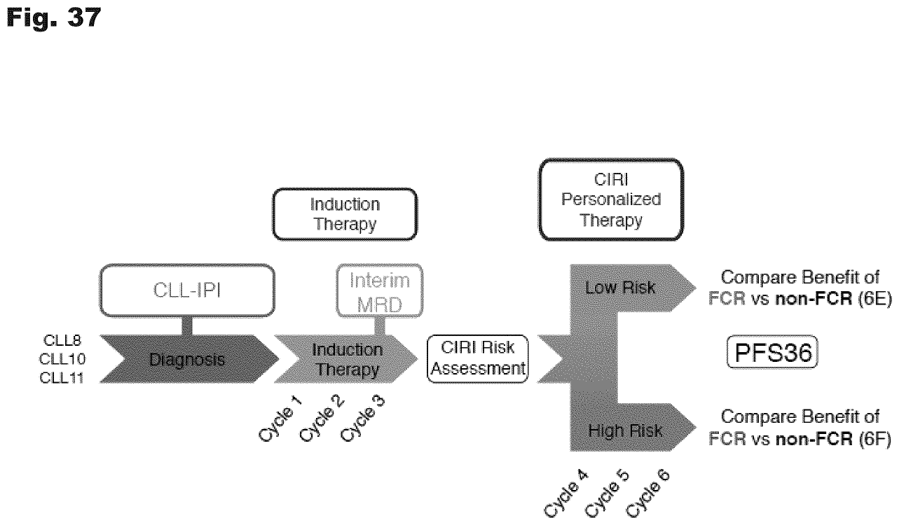

Taken as a single biomarker (FIG. 35), interim MRD was prognostic, but not predictive of benefit from subsequent therapy. Specifically, MRD negative (MRD−) patients had superior outcomes to MRD positive (MRD+) patients; yet, both MRD− and MRD+ patients benefited from FCR over alternatives (FIG. 36). In contrast, CIRI-CLL provides each individual patient with a quantitative estimate of the probability of disease progression at 36 months, based on the combination of CLL-IPI and interim MRD (FIG. 37). Therefore, a range of thresholds are available to separate patients into low- and high-risk groups. CIRI-CLL establishes parameters for the benefit from each alternative therapy—this allows a forecast of the outcomes for patients receiving each potential therapy.

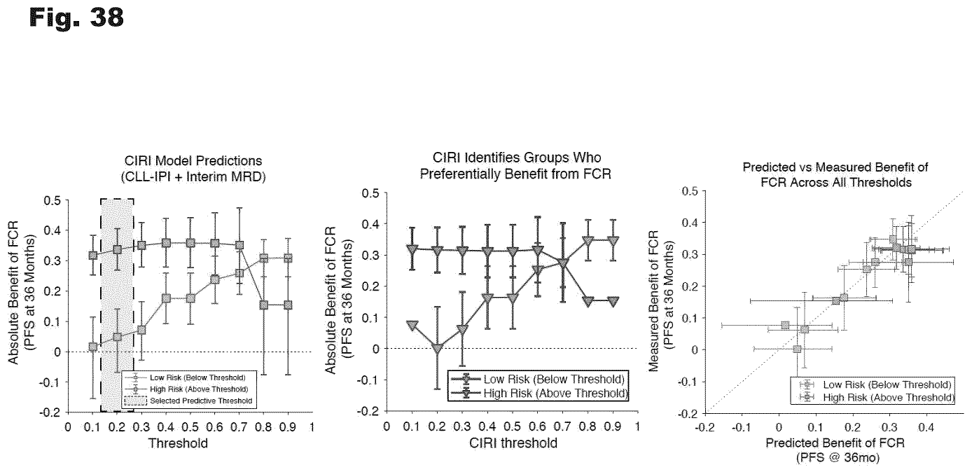

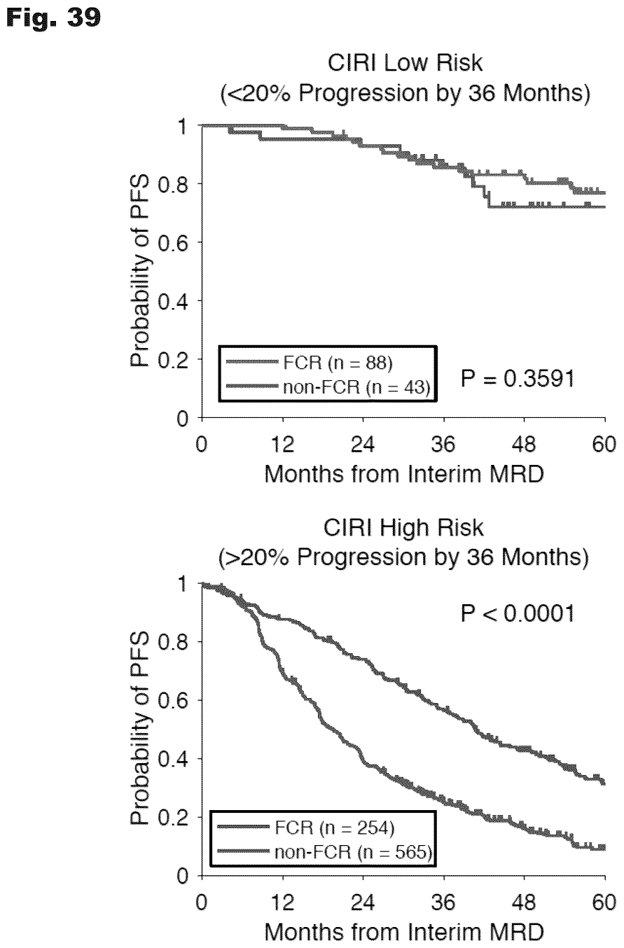

This forecasting was performed to estimate the likely benefit from FCR versus non-FCR therapy for groups of high vs low risk patients defined by CIRI at various thresholds (FIG. 38). Interestingly, while patients with a probability of progression >20% at 36 months were predicted to benefit from FCR, patients with <20% probability of progression were not predicted to benefit. More importantly, these predictions were confirmed in patient outcomes (FIG. 39)—patients with >20% probability of progression at 36 months significantly benefited from FCR therapy, while patients with <20% probability of progression did not. This differential effect between low- and high-risk patients indicates that CIRI-CLL is a predictive biomarker. Furthermore, the interaction between CIRI and choice of therapy was statistically significant for prediction of PFS at 36 months in multivariate models (Cox proportional hazards, P=0.047; generalized linear model, P=0.0006), further indicating a predictive biomarker. As compared to interim MRD, the improvement in predictive power by CIRI stems primarily from reclassifying MRD− patients. In total, 31% (60/191) of MRD− patients were reclassified as CIRI high-risk, due to high or very-high CLL-IPI scores. When comparing the predicted benefit of FCR versus alternate therapies from THE model to the observed benefit at each threshold, the observed benefit of FCR in low- and high-risk populations largely agreed with the model predictions (FIG. 38).

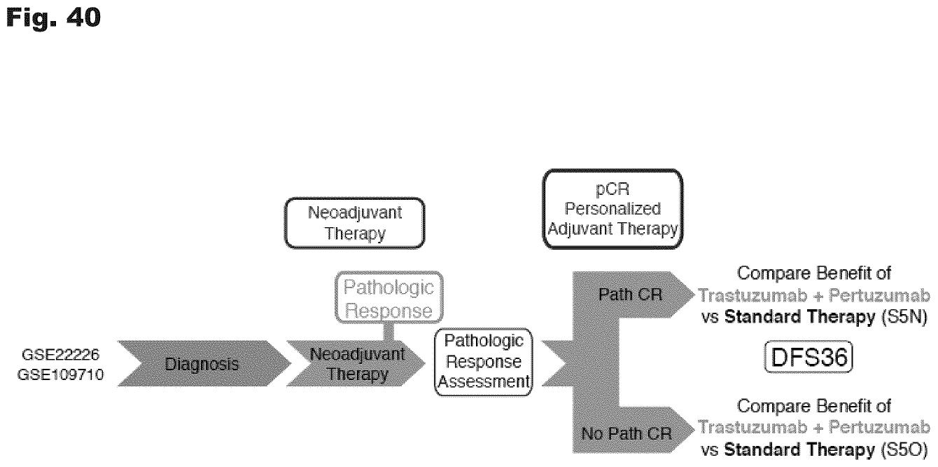

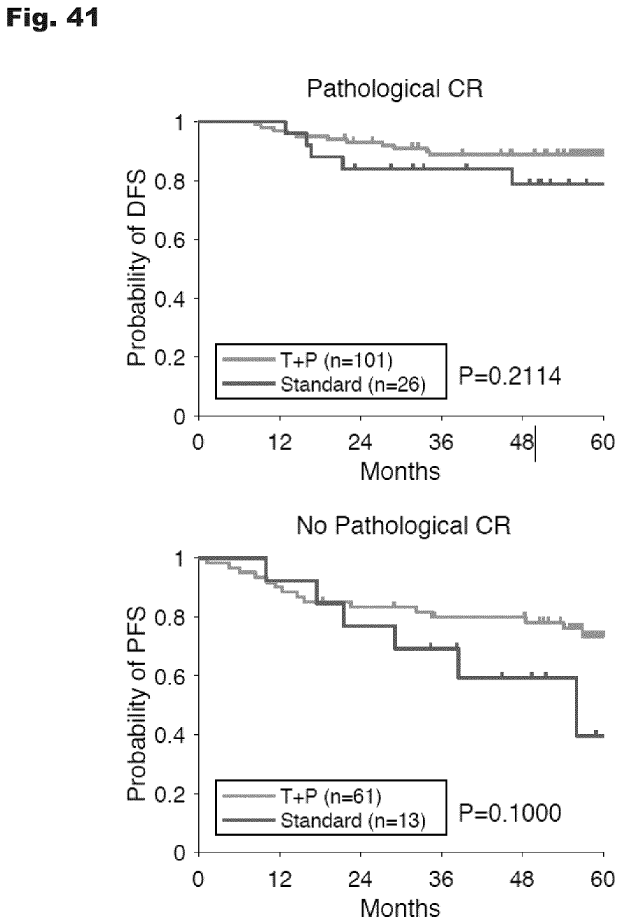

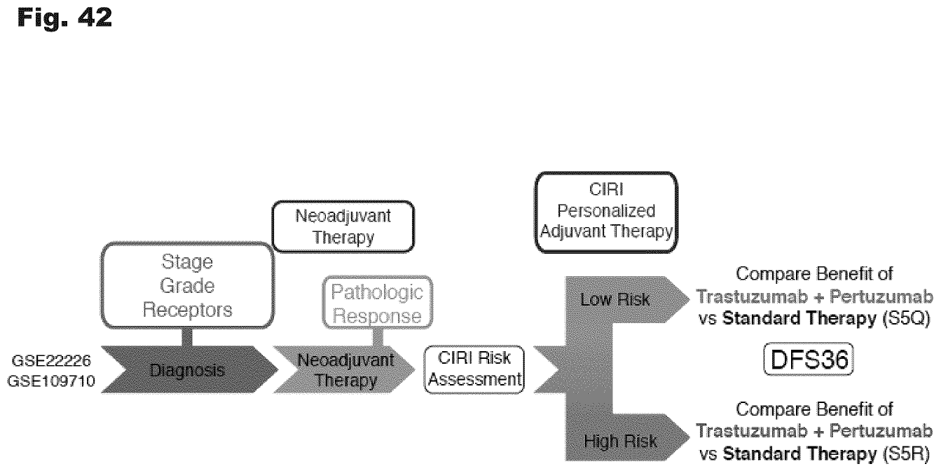

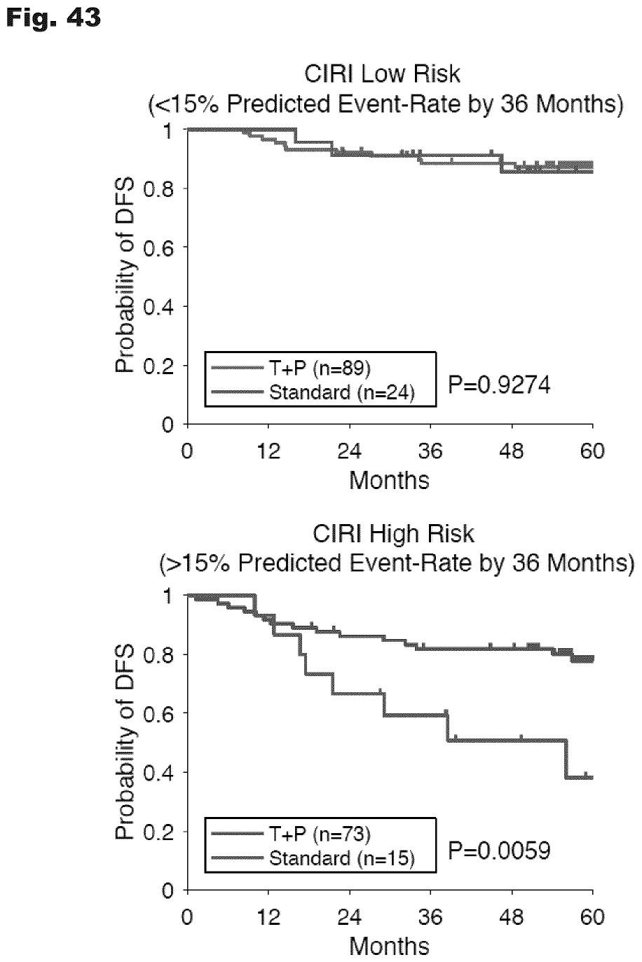

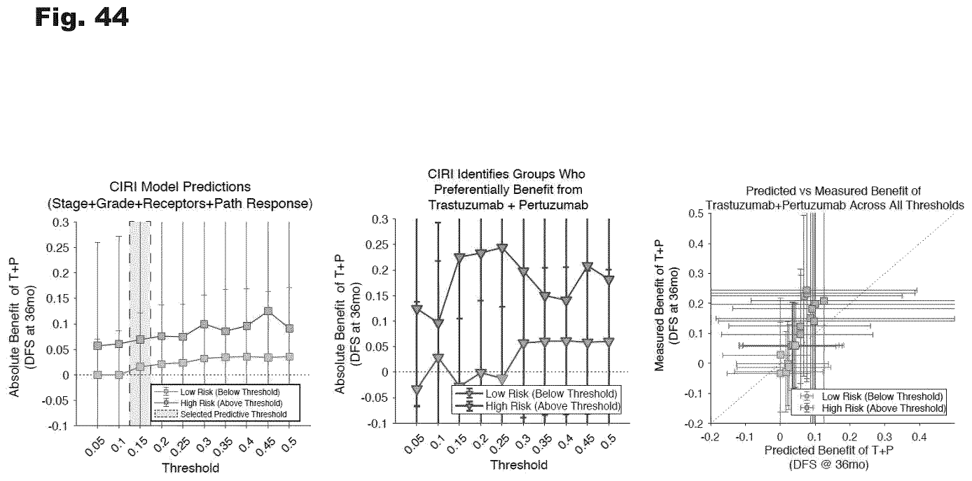

The ability of CIRI was further explored to identify predictive biomarkers in the context of neoadjuvant chemotherapy for locally advanced HER2+ breast adenocarcinoma. The CIRI-BRCA model was applied to publicly available retrospective data from multiple clinical trials where neoadjuvant therapy was given (L. J. Esserman, et al., Breast Cancer Res Treat 132, 1049-1062 (2012); and M. Ignatiadis, et al., J Natl Cancer Inst 111, 69-77 (2019); the disclosures of which are each incorporated herein by reference). Analysis was limited to only patients with HER2+ disease to examine the effect of dual HER2-targeted therapy with Trastuzumab and Pertuzumab. Similar to interim MRD in CLL, pathologic response to therapy was not predictive of benefit from dual HER2 therapy (FIGS. 40 and 41). In contrast, CIRI-BRCA with a threshold of > or <15% probability of progression identified a group of high-risk patients who preferentially benefited from dual targeted therapy (FIGS. 42, 43, and 44).

### Discussion

Over the last two decades, numerous prognostic biomarkers have been described throughout oncology, with particular emphasis on pretreatment clinical and molecular factors. Various statistical methods to integrate these biomarkers at a fixed time-point have been described resulting in clinically useful prognostic scores for a variety of cancers. Such fixed time-point risk models are routinely used to determine prognosis at multiple clinical landmarks, including at the time of initial diagnosis or at the time of second-line or salvage therapy. Importantly, these models generally do not consider dynamic response to therapy as a feature. More recently, evoked biomarkers capturing a phenotypic response to therapy have been described using radiographic, pathologic, or molecular features. While these features are independently prognostic of outcomes, it was postulated that integration with other predictors-including pretreatment factors-would improve and individualize outcome predictions.

Early heuristic efforts to combine response-based biomarkers with pretreatment factors have shown promise for improving outcome prediction. Moreover, physicians routinely utilize anatomical imaging, blood work, and clinical symptoms to monitor patients during therapy and qualitatively assess risk. Unfortunately, in the clinical care of individual patients, evoked and dynamic risk-factors do not easily lend themselves to multivariate analysis due to their collection over time and the possibility of missing data-points. Prior methods for predictive modeling with covariates that change over time based on proportional hazard models have been explored (L. D. Fisher and D. Y. Lin, Annu Rev Public Health 20, 145-157 (1999), the disclosure of which is incorporated herein by reference); however, these often require complex functional forms and have difficulty dealing with missing data. The emergence of novel response-based biomarkers, along with the long-standing use of routine clinical testing during a patient's treatment course, affords an opportunity to improve on existing prognostic tools by integration of diverse dynamic data.

To overcome these shortcomings CIRI was developed, a method to integrate diverse outcome predictors collected over time, resulting in a quantitative, personalized prediction of clinical outcomes. Here, two different approaches were utilized—an initial naïve Bayes method to predict outcomes at a fixed endpoint, and a method based on Bayesian analysis and proportional hazard assumptions to develop personalized predictions of outcomes over time. In each method, the model is updated as information is gathered over a disease course. Notably, CIRI is distinct from machine-learning approaches in two key aspects. First, rather than starting with a large number of possible features, CIRI leverages prior knowledge and utilizes only a handful of established risk-factors. Second, by limiting the number of predictors and applying Bayesian frameworks, a prognostic model can be constructed by establishing a small number of parameters. These parameters remove the need for a large number of training cases. Indeed, the performance of CIRI was superior to proportional hazard modeling when the number of training cases available for model development was limited. This scenario is often encountered with emerging biomarkers such as liquid biopsies. Consequently, by estimating the prognostic impact of novel risk-factors from relatively limited external data, CIRI can be easily updated as new biomarkers emerge.

To demonstrate the performance of this method, CIRI models were developed to predict outcomes in three different malignancies, when using a diverse source of biomarkers to measure response to therapy. Specifically, serial ctDNA levels were evaluated as a measure of residual disease in a common aggressive lymphoma (DLBCL), minimal residual disease from circulating cells in a common leukemia (CLL), and histopathological evidence of microscopic residual cancer in resected breast tumors following neoadjuvant therapy. In each case, CIRI produces a personalized, quantitative prediction of the probability of clinically relevant outcome that is updated as more information is made available. In independent validation cohorts, a sufficient degree of calibration was demonstrated in all three diseases—that is, the predicted probability of outcome produced by CIRI is close to the observed data. Calibration is an important consideration for any predictive model, and it is worth noting that the model with the most training and validation cases (CIRI-CLL) demonstrated the best calibration.

Importantly, CIRI demonstrated superior outcome prediction to current gold standard prognostic indices, including against validated risk models tailored for each disease. Indeed, the composite CIRI model in each disease improved on each individual component predictor by C-statistic, demonstrating the importance of considering a diverse source of data when making predictions.

CIRI was evaluated in patient cohorts with some element of case-level missing data; that is, not every risk-factor considered by CIRI was available for every patient. The fact that CIRI remains robust to missing data when making predictions for individuals is one of its key features, as missing data is commonplace in the clinic. Moreover, the performance of CIRI would only improve in cases where complete data are available.

When applying CIRI and other risk models, attention must be paid to the clinical scenario and specifically the choice of therapy. The performance of risk models trained in the context of a given therapy may degrade as more effective novel therapies emerge. Correspondingly, in the case of CLL, where the choice of frontline therapy can significantly impact expected PFS and OS, the choice of therapy was explicitly considered as an outcome predictor. As new therapies emerge, the effect of these on outcomes should be considered and integrated into CIRI. Doing so should maximize the discriminative performance of CIRI, although prognostication in a therapy-independent context may also be valuable.

The possibility of using quantitative risk assessment through CIRI was explored as an alternative approach for developing predictive biomarkers. In a proof-of-concept analysis in CLL utilizing CLL-IPI and interim MRD, a high-risk group of patients who preferentially benefited from aggressive immunochemotherapy with FCR was identified, as well as a low-risk group of patients who did not benefit from FCR over other evaluated therapies. A similar analysis in HER2+ breast adenocarcinomas revealed a group of high-risk patients who preferentially benefited from dual HER2-targeted therapy. These unplanned post hoc analyses have important limitations—most notably that selecting therapy based on CIRI requires a period of ‘induction therapy’ prior to personalized therapy selection. Most historic trials have not been performed in this manner, requiring blinding to choice of initial therapy in this proof-of-concept. Nevertheless, similar survival outcomes have been observed in scenarios where systemic treatment can be performed before or after surgery (e.g., adjuvant vs. neoadjuvant therapy for breast cancer), suggesting the feasibility of this approach. This data suggests a shift in clinical paradigms to include an initial ‘induction phase’ where response to therapy can be assessed could facilitate a new class of predictive biomarkers.

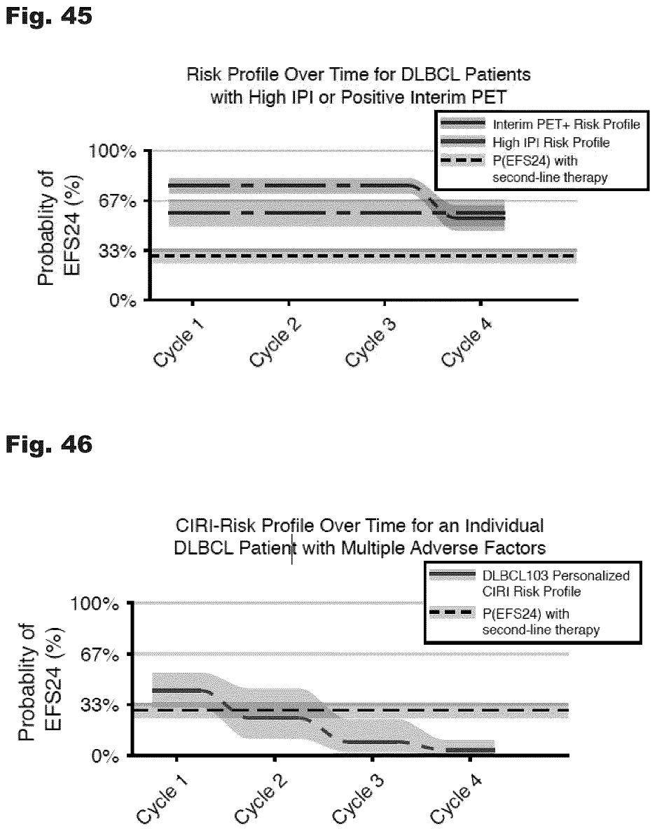

CIRI furthermore provides a potential path forward to aid clinical decision-making by providing quantitative estimates of likely outcomes. To illustrate this, consider a current clinical challenge-selecting high-risk DLBCL patients for intensified therapy. While previous methods to select patients for early treatment intensification, such as the IPI and interim PET/CT scans, are able to identify a group with worse outcomes than the average patient, prior studies intensifying therapy for patients based on these factors alone have failed to improve overall survival. This is potentially due to largely favorable outcomes for patients despite high-risk IPI or interim PET/CT scan, particularly in comparison to the efficacy of salvage therapy. Indeed, 42% of patients with positive interim PET/CT scans remain disease-free at 24 months with continued RCHOP, compared to ~30% EFS24 rate in unselected patients receiving salvage therapy. In contrast, CIRI considers the probability of outcome for each patient individually (FIG. 45); by doing so, CIRI can identify individual patients with extremely high-risk of treatment failure who are unlikely to benefit from their current treatment as compared to possible salvage approaches (FIG. 46). While this method does inherently identify a smaller fraction of patients than current approaches, identifying small groups of individual patients likely to benefit from alternative therapy is beneficial to implement personalized approaches.

In total, the data suggest that identification of personalized risk throughout a course of therapy is feasible and can improve upon established risk assessment tools. Dynamic risk modeling will facilitate novel clinical trial designs for personalized therapeutic approaches, with wide applicability in oncology and other areas of medicine.

### Methods

### DLBCL Patient Data Collection

Data were analyzed from subjects with large B-cell lymphomas undergoing treatment at six institutions across North America and Europe with serial ctDNA measurements available. A total of 181 patients receiving anthracycline-based immunochemotherapy were enrolled at Stanford University (Stanford, CA, USA, n=49), MD Anderson Cancer Center (Houston, TX, USA, n=23), University of Eastern Piedmont (Novara, Italy, n=36), the National Cancer Institute (Bethesda, MD, USA, n=33), Essen University Hospital (Essen, Germany, n=15), and Center Hospitalier Universitaire Dijon (Dijon, France, n=25). Patients in this study were prospectively enrolled and provided written, informed consent. Levels of ctDNA were measured prior to the first, second, and third cycles of therapy as previously described (A. M. Newman, et al., Nature medicine 20, 548-554 (2014); and F. Scherer, et al., Science translational medicine 8, 364ra155 (2016), the disclosures of which are each incorporated herein by reference). Circulating tumor DNA measurements were used to predict EFS as previously described (D. M. Kurtz, et al., (2018), cited supra).

### CIRI-DLBCL Model Development and Validation

To build CIRI-DLBCL, prior knowledge on the performance of included risk factors is required to determine the model parameters (for details on the necessary parameters, see “Design details of the Continuous Integrated Risk Index” section of the STAR Methods). CIRI-DLBCL considers a total of six risk factors, including the International Prognostic Index (IPI), molecular cell of origin, interim imaging, along with ctDNA measurements prior to cycles one, two, and three of therapy. Estimates for the prior probability of event-free survival for the average patient with DLBCL were obtained, as well as the parameters for CIRI-DLBCL from previously established literature describing the IPI (L. H. Sehn, et al., (2007), cited supra; and M. Ziepert, et al., (2010), cited supra), cell of origin (D. W. Scott, et al., (2015), cited supra), and interim imaging procedures (A. F. Cashen, et al., (2011), cited supra; I. N. Micallef, et al., (2011), cited supra; N. Nols, et al., (2014), cited supra; P. Pregno, et al., (2012), cited supra; V. Safar, et al., (2012), cited supra; D. H. Yang, et al., (2011), cited supra; C. Yoo, et al., (2011), cited supra; and P. L. Zinani, et al., (2011), cited supra). To determine the performance of ctDNA for predicting patient outcomes and the corresponding parameters, the 49 patients from Stanford University were utilized as a development set. The performance of CIRI-DLBCL was tested in the validation set consisting of the remaining 132 patients.

### CLL Patient Data Collection

Patient level clinical and peripheral blood MRD data were reanalyzed; the data was derived from patients enrolled in three phase III clinical trials from the German CLL Study Group (GCLLSG)—CLL8: fludarabine and cyclophosphamide (FC) vs. FC plus rituximab (FCR); CLL10: FCR vs. bendamustine plus rituximab (BR); and CLL11: chlorambucil vs. chlorambucil plus rituximab vs. chlorambucil plus obinutuzumab (NCT00281918, NCT00769522, and NCT02053610) (B. Eichhorst, et al., (2016), cited supra; V. Goede, et al., (2014), cited supra; and M. Hallek, et al., (2010), cited supra). Trial protocols were approved by the relevant institutional review board and ethics committee of each participating center. Patients provided written informed consent to participate in the trials and to undergo MRD testing. A total of 1426 patients with either interim or final MRD measurement from peripheral blood were available. Interim MRD measurement was obtained after the first 3 cycles of therapy; final MRD measurement was obtained 3 months after the completion of therapy. MRD was measured either by four-color flow cytometry (CLL8 and CLL10) or by polymerase chain reaction (PCR, CLL11). MRD negativity was defined as a level of <10-4; this threshold was previously found to be predictive for both progression-free and overall survival (S. Bottcher, et al., (2012), cited supra).

### CIRI-CLL Model Development and Validation

To build and parameterize CIRI-CLL, patients were divided into separate development and validation sets. Patients were randomly assigned to either the development or validation set with a 50% chance of assignment to either group. CIRI-CLL considers four risk factors, including choice of first-line therapy, CLL-IPI (The International CLL-IPI Working Group, (2016), cited supra), interim MRD, and final MRD. The prior probability of progression free survival and corresponding parameters for each risk factor was determined from the development set (n=699), providing the parameters for CIRI-CLL. This model was then evaluated in the independent validation set (n=727).

### Breast Adenocarcinoma Patient Collection

Case-level data were obtained from a previous study of taxane and anthracycline based neoadjuvant chemotherapy for patients with resectable breast adenocarcinoma (GEO series GSE25066) (C. Hatzis, et al., (2011), cited supra). Patients in this study were prospectively enrolled and provided written, informed consent. Patients were treated with neoadjuvant anthracycline and taxane based chemotherapy, followed by surgical resection. Pathological response to chemotherapy was assessed using the residual cancer burden method as previously described (W. F. Symmans, et al., Journal of clinical oncology: official journal of the American Society of Clinical Oncology 25, 4414-4422 (2007), the disclosure of which is incorporated herein by reference). A total of 417 patients with clinical data were available. To explore the possibility of using CIRI to discover predictive biomarkers, CIRI-BRCA was assessed in two additional publicly available cohorts of patients with HER2+ breast adenocarcinoma in the context of neoadjuvant therapy (L. J. Esserman, et al., (2012), cited supra; and M. Ignatiadis, et al., (2019), cited supra) (GEO series GSE22226 and GSE109710).

### CIRI-BRCA Model Development and Validation

To build CIRI-BRCA, prior knowledge and the model parameters from were established two independent, previously published studies. CIRI-BRCA considers four separate risk factors—clinical stage, tumor grade, estrogen receptor/HER2 status, and pathological response to chemotherapy. The prior probability of survival, as well as the parameters based on stage, grade, and receptor status, were all determined from patient-level data from the METABRIC study (C. Curtis, et al., (2012), cited supra). The likelihood of distant-relapse free survival based on pathological response to chemotherapy was derived from a separate, previously published study (W. F. Symmans, et al, (2017), cited supra). These values determined the parameters for the CIRI-BRCA model. The performance of CIRI-BRCA was tested in the validation set of 417 patients receiving neoadjuvant chemotherapy described above, which was independent of the patients used to parameterize the model.

### Design Details of the Continuous Individualized Risk Index

Two separate approaches to dynamic risk modeling, collectively described as CIRI, are provided within. The first utilized a naïve Bayesian framework to estimate the probability of a clinical outcome at a defined endpoint in time. This initial approach is used to describe the concept of CIRI, with data shown for CIRI-DLBCL. The second method estimates a personalized probability of survival over time (i.e., a predicted survival curve) based on Cox proportional hazard modeling, and is used to construct the final CIRI-DLBCL model as well as CIRI-CLL and CIRI-BRCA. Each of these two methods is described in the sections below.

### CIRI for Fixed-Endpoints

A naïve Bayesian framework was used to predict the risk of clinical events at defined endpoints in time. To construct a CIRI model in this framework, a number of parameters need to be identified. These include an initial probability of adverse outcome, P(event), which is applicable to the patient population without knowledge of any risk factors. Additionally, for a set of risk factors, {f₁, f₂, . . . fₙ}, the conditional probabilities P(fᵢ|event) and P(fᵢ|no event) need to be determined as well. For details on the determination of these parameters for CIRI-DLBCL, please see the section on “Determination of model parameters in CIRI for fixed-endpoints” below.

Using these conditional probabilities, prognostic features were applied for each patient to a baseline estimate via Bayes' Theorem:

$$P ( event \vert feature ) = \frac{P ( event ) * P ( feature \vert event )}{P ( feature )}$$

$$where$$

$$P ( feature ) = P ( event ) * P ( feature \vert event ) + ( 1 - P ( event ) ) * P ( feature \vert no event )$$

and P(event) is the prior probability of an event. Sequential prognostic features were added to determine the personalized probability of an event for each patient with increasing amounts of information.

Extending this framework to patients with multiple prognostic features, {f₁, f₂, . . . fₙ}:

$$P ( event \vert {f}_{1} , {f}_{2} , … {f}_{n} ) ∝ P ( {f}_{1} , {f}_{2} , … {f}_{n} \vert event ) * P ( event )$$

The naïve Bayes approach assumes independence of the features within each event or no-event group. Under these independent assumptions:

$$P ( {f}_{1} , {f}_{2} , … {f}_{n} \vert event ) = P ( {f}_{1} \vert event ) * P ( {f}_{2} \vert event ) * … * P ( {f}_{n} \vert event ) = {\prod }_{i} P ( {f}_{i} \vert event )$$

$$Therefore ,$$

$$P ( event \vert {f}_{1} , {f}_{2} , … {f}_{n} ) ∝ P ( event ) * {\prod }_{i} P ( {f}_{i} \vert event )$$

Converting this proportionality to a probability results in:

$$P ( event \vert {f}_{1} , {f}_{2} , … {f}_{n} ) = \frac{P ( event ) * {\prod }_{i} P ( {f}_{i} \vert event )}{P ( event ) * {\prod }_{i} P ( {f}_{i} \vert event ) + P ( no event ) * {\prod }_{i} P ( {f}_{i} \vert no event )}$$

This equation is mathematically equivalent to the naïve Bayes approach outlined above.

For CIRI, prognostic features are sequentially added, as information becomes available. For example, in the case of CIRI-DLBCL, the following prognostic features available prior to therapy are first added, with the allowed values for each feature listed in curly brackets: 1) International Prognostic Index {low, low-intermediate, high-intermediate, high, N/A}; 2) pretreatment ctDNA {low, high, N/A}; 3) cell-of-origin {GCB, non-GCB, N/A}. After the first cycle of therapy, cycle 2 ctDNA becomes available, with possible values {EMR, No EMR, N/A}. After the second cycle of therapy, cycle 3 ctDNA becomes available. {MMR, no MMR, N/A}. Finally, interim imaging measurements become available {residual disease, no residual disease, N/A}. Note that when data was missing or not available, the prognostic feature was considered non-informative and the prior probability was not updated.

To make the details clearer, the following example illustrates CIRI probabilities over time. Consider a patient, Patient B from FIG. 3, with a diagnosis of DLBCL. Without any information about his clinical features, his initial (prior) probability of event by two years (e.g.: P(event)) is estimated to be 21.8%, based on literature values for the disease (Table 2 in FIG. 5B). Now, it is learned that his IPI is 3, putting him in the high intermediate risk group. His probability of adverse event is therefore updated to:

$$P ( event \vert IPI = 3 ) = \frac{P ( event ) * P ( IPI = 3 \vert event )}{P ( IPI = 3 )}$$

which is equivalent to

$$P ( event \vert IPI = 3 ) = \frac{P ( event ) * P ( IPI = 3 \vert event )}{P ( event ) * P ( IPI = 3 \vert event ) + ( 1 - P ( event ) ) * P ( IPI = 3 \vert no event )}$$

all the probabilities on the right-hand side of this equation from can be obtained from the model parameters (Table 2 in FIG. 5B), resulting in:

$$P ( event \vert IPI = 3 ) = \frac{0.218 * 0.305}{0.218 * 0.305 + ( 1 - 0.218 ) * 0.129} = 0.40$$

or a 40% probability of relapse. Now suppose that prior to therapy, this patient has not one, but three risk-predictors-IPI=3, low ctDNA, and GCB molecular subtype. His full expression for risk of an event is therefore:

$$P ( event \vert IPI = 3 , low ctDNA , GCB ) = \frac{P ( event ) * A}{P ( event ) * A + P ( no event ) * B}$$

$$Where :$$

$$A = {\prod }_{i} P ( {f}_{i} \vert event ) = P ( IPI = 3 \vert event ) * P ( low ctDNA \vert event ) * P ( GCB \vert event ) = ( 0.305 ) * ( 0.235 ) * ( 0.602 ) = 0.042$$

$$And$$

$$B = {\prod }_{i} P ( {f}_{i} \vert no event ) = P ( IPI = 3 \vert event ) * P ( low ctDNA \vert no event ) * P ( GCB \vert no event ) = ( 0.129 ) * ( 0.563 ) * ( 0.369 ) = 0.027$$

$$Therefore ,$$

$$P ( event \vert IPI = 3 , low ctDNA , GCB ) = \frac{0.218 * 0.042}{0.218 * 0.042 + ( 1 - 0.218 ) * 0.027} = 0.148$$

which is equivalent to a 14.8% chance of event by 24 months, or an 85.2% probability of reaching EFS24, as shown in FIG. 3.

After one cycle of therapy, however, the patient does not achieve an early molecular response (no EMR, second time-point, FIG. 3). Upon learning this information, his expression for probability of event is:

$$P ( event \vert IPI = 3 , low ctDNA , GCB , No EMR ) = \frac{P ( event ) * A}{P ( event ) * A + P ( no event ) * B}$$

Where A and B are now:

$$A = {\prod }_{i} P ( {f}_{i} \vert event ) = P ( IPI = 3 \vert event ) * P ( low ctDNA \vert event ) * P ( GCB \vert event ) * P ( No EMR \vert event ) = ( 0.305 ) * ( 0.235 ) * ( 0.602 ) * ( 0.429 ) = 0.0027$$

$$And$$

$$B = {\prod }_{i} P ( {f}_{i} \vert no event ) = P ( IPI = 3 \vert event ) * P ( low ctDNA \vert no event ) * P ( GCB \vert no event ) * P ( No EMR \vert no event ) = ( 0.129 ) * ( 0.563 ) * ( 0.369 ) * ( 0.19 ) = 0.0068$$

$$Therefore ,$$

$$P ( event \vert IPI = 3 , low ctDNA , GCB , No EMR ) = \frac{0.218 * 0.0027}{0.218 * 0.0027 + ( 1 - 0.218 ) * 0.0068} = 0.282$$

which is equivalent to a 28.2% chance of event by 24 months, or a 71.8% probability of reaching EFS24, as shown in FIG. 3 (second step).

Subsequent measurements of ctDNA and interim imaging studies for this patient further update his probability of relapse. Note that for these subsequent measurements (e.g.: EMR, MMR, and interim imaging), the two-year event probability is still estimated for the patient from the time of diagnosis. That is, the fact that the patient has been relapse-free for 21 days at cycle 2 is not taken into account. This will suffer from some guarantee-time bias, which may cause an over-estimate the relapse probability. However, this bias is small, as evidenced by the calibration curve (see FIG. 6).

An example of CIRI for fixed endpoint for two patients is given in FIG. 3. The expectation value for the probability of event-free survival at 24 months is shown as a solid line for each patient. The expectation value from CIRI was used. A distribution of the posterior probability and confidence intervals can additionally be obtained. To perform this, the variance for each baseline and conditional probability used in CIRI was estimated (using Greenwood's formula). A distribution of the posterior probability was then determined by sampling from the distribution of each conditional and prior probability and performing the naïve Bayes analysis 10,000 times (Markov Chain Monte Carlo). This distribution was used to create the 80% confidence intervals shown in FIG. 9. This posterior distribution was also used to in the proposed framework for therapy selection utilizing CIRI shown in FIGS. 45 and 46. (see “Framework for therapeutic selection from CIRI risk estimates” section of the methods).

### Determination of Model Parameters in CIRI for Fixed-Endpoints

The goal of fixed-endpoint CIRI is to estimate the probability of adverse event for a given patient at a fixed point in time, given a set of n features (i.e., P(event|f₁ . . . fₙ)). To use the approach outlined above, a number of parameters need to be determined. These include an initial or baseline probability of adverse outcome, P(event), applicable to an entire patient population at large, as well as two conditional probabilities—P(fᵢ|event) and P(fᵢ|no event)—for all prognostic features fᵢ of interest.

To estimate the baseline probability of an event, P(event), Kaplan-Meier estimates of the survival function were employed. This was used to estimate the probability of survival, or ‘no event’, P(no event), with the knowledge that P(event)=1−P(no event). When patient level survival data was available, Kaplan-Meier survival estimates were constructed from this primary data. When patient level survival data was not available, survival functions were estimated from the literature (see methods section on “Estimation of survival functions from published literature”).

To determine the conditional probabilities P(fᵢ|event) and P(fᵢ|no event), a similar approach was employed. To identify these parameters for a given feature, F with possible values {f₁, f₂, . . . fᵢ . . . fₙ}, beginning with Kaplan-Meier survival estimates of subgroups of interest—i.e., P(no event|fᵢ). Using this survival function, P(no event|fᵢ) and P(event|fᵢ) was determine at the end-point of interest:

$$P ( event \vert {f}_{i} ) = 1 - P ( no event \vert {f}_{i} )$$

The number of patients with and without events is estimated as follows:

$${n}_{( {f}_{i} , event )} = {n}_{{f}_{i}} * P ( event \vert {f}_{i} )$$

$${n}_{( {f}_{i} , no event )} = {n}_{{f}_{i}} * P ( no event \vert {f}_{i} )$$

Where n_{f}_{ᵢ} is the number of patients with a given risk feature. With this information, an estimate can be calculated:

$$P ( {f}_{i} \vert event ) = \frac{{n}_{( {f}_{i} , event )}}{{\sum }_{i} {n}_{( {f}_{i} , event )}}$$

$$P ( {f}_{i} \vert no event ) = \frac{{n}_{( {f}_{i} , no event )}}{{\sum }_{i} {n}_{( {f}_{i} , no event )}}$$

For example, see FIG. 4A, where these parameters were estimated for the IPI. Here, to estimate:

$$P ( no event \vert low IPI ) = 0.891$$

$$P ( no event \vert low int IPI ) = 0.777$$

$$P ( no event \vert high int IPI ) = 0.617$$

$$P ( no event \vert high IPI ) = 0.599$$

$$Therefore ,$$

$${n}_{( low , no event )} = 553 * 0.891 = 492.7$$

$${n}_{( low int , no event )} = 227 * 0.777 = 176.4$$

$${n}_{( low , no event )} = 175 * 0.617 = 108.$$

$${n}_{( low , no event )} = 105 * 0.599 = 62.9$$

$$Finally ,$$

$$P ( low IPI \vert no event ) = \frac{492.7}{492.7 + 176.4 + 108. + 62.9} = 0.587$$

Conditional probabilities for the other IPI groups and for patients having adverse events are calculated similarly.

### CIRI for Survival Analysis

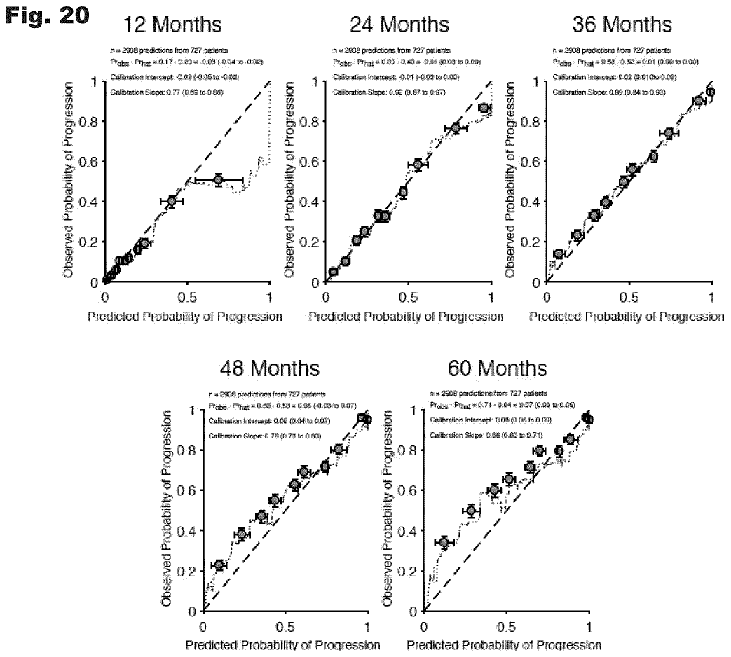

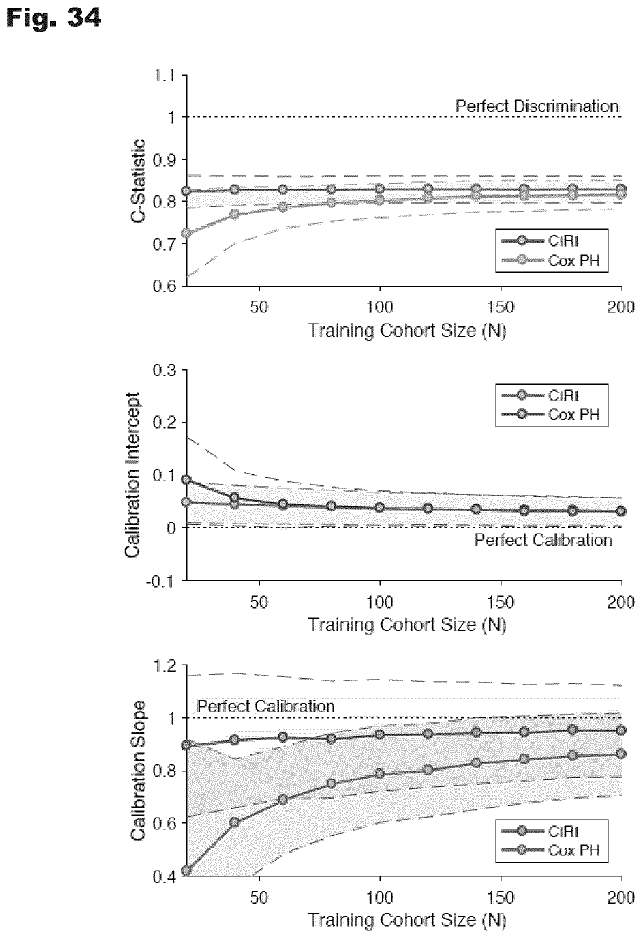

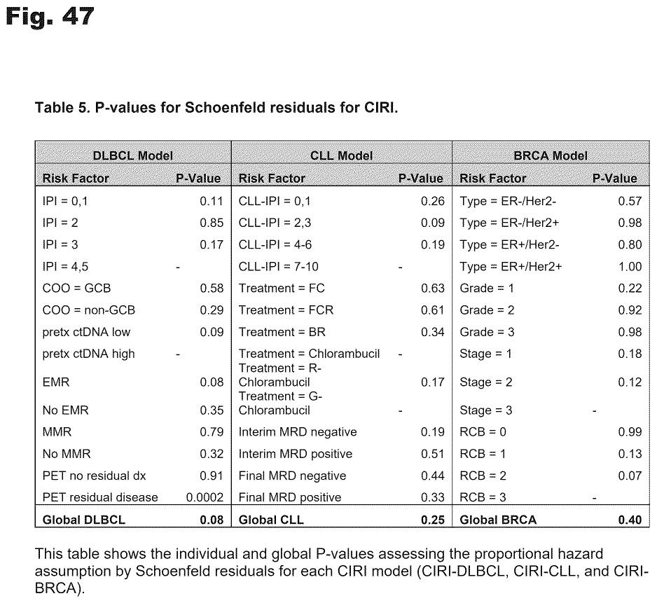

The concept for dynamic risk-prediction was extended to including prediction of a personalized survival function over time by utilizing the Cox proportional hazard model. This method was utilized to produce the results shown in FIGS. 9-47. The use of the proportional hazard assumption in CIRI-DLBCL, CIRI-CLL, and CIRI-BRCA was largely satisfied-after constructing a standard Cox proportional hazard model for each disease, the proportional hazard assumption was tested by Schoenfeld residuals, resulting in global P-values for the composite model of 0.08, 0.25, and 0.40 respectively. The individual P-values for the test of proportional hazards for each individual covariate are provided in Table 5 (FIG. 47). In the case of singularity (i.e., IPI values of {0-1,2,3,4-5}), one value has been removed from analysis to allow model convergence.

Having assessed the proportional hazard assumption, model parameters were identified for CIRI. In an ordinary Cox model, a baseline survival function is utilized, denoted by S₀(t), a set of observed covariates, denoted by X, and a set of (regression) coefficients, β reflecting the deviation from the baseline survival given the observed covariates. These will lead to the final survival function

$$S ( t ) = {{S}_{0} ( t )}^{exp ( {X}^{T} β )} .$$

In this setting, the coefficients are often inferred, via maximum partial likelihood estimation, from a set of samples with joint covariate information, in which the accuracy of the estimated coefficients is highly dependent on the number of available samples. Moreover, although the inference can be done in the presence of missing values, the performance can significantly degrade.

In most cases with clinical survival data, the joint covariate information is extremely rare, due to either limited sample sizes or the absence of case-level reporting in the literature. In contrast, the univariate survival information can be straightforwardly extracted from the literature (see section on “estimation of survival functions from published literature”), which can be incorporated, as prior knowledge, into the survival prediction. Therefore, a “Bayesian Cox model” was utilized, where Cox coefficients are governed by some “prior probability.” A natural choice for such prior probability is normal distribution, and therefore β~π(β)=N(μ, Σ). Due to the type of prior knowledge, it was assumed Σ to be a diagonal matrix. It should be noted that Σ can be a non-diagonal matrix if there is joint prior information (for a relatively large cohort).

Taking a Bayesian approach, final survival function is defined as

$${S}^{*} ( t ; X ) = \int {{S}_{0} ( t )}^{exp ( {X}^{T} β )} π ( β ) d β$$

where π(β) is the prior probability of β. The Cox partial likelihood was used as the likelihood function, and Markov Chain Monte Carlo (MCMC) sampling was employed to calculate the posterior survival function for each individual patient. In the cases where no sample is used for posterior calculation, it basically reduces to a model averaging schema.

### Parameter Construction in CIRI for Survival Analysis

In order to estimate the mean and variance of the priors for the Bayesian Cox model, the uncertainty around the prior survival curves was used, as estimated by the total number of patients at risk at a given time point (see “Estimation of survival functions from published literature”). Therefore, the input to this step was a set of survival curves along with their confidence intervals. It was assumed that all the covariates are converted into binary valued variables, i.e. dummy variables. The hyper-parameters were inferred via an optimization framework which fits the final prior probability to the given curves.

Mathematically, Pr[S₀(t)^{exp(β)}∈[L_{b}, U_{b}]]≈1−α is satisfied with α being a user parameter (α=0.05 is used in CIRI) and β~N(μ, σ²). Note that here, the function S₀(t), the survival of an unselected population of patients, is determined from prior knowledge derived from the literature as described in the “Estimation of survival functions from published literature” section. Here, L_{b} and U_{b} are lower and upper bounds for the effect of individual risk-factors (i.e., beta-value). These bounds are estimated using the uncertainty of survival given each individual risk-factor using Greenwood's formula (M. Greenwood, A report of the natural duration of cancer (London: H. M. Stationery off.) (1926), the disclosure which is incorporated herein by reference.

The task is then to find μ and σ (here and for simplicity in notations the subscript indexing the covariates was dropped). Hence, first the probability was simplify as follows:

$$Pr [ {{S}_{0} ( t )}^{exp ( β )} ∈ [ {L}_{b} ( t ) , {U}_{b} ( t ) ] ] = Pr [ {e}^{β} log {S}_{0} ( t ) ∈ [ log {L}_{b} ( t ) , log {U}_{b} ( t ) ] ] = Pr [ log - {e}^{β} log {S}_{0} ( t ) ∈ [ log ( - log {U}_{b} ( t ) ) , log ( - log {L}_{b} ( t ) ) ] ] = Pr [ log {e}^{β} + log ( - log {S}_{0} ( t ) ) ∈ [ log ( - log {U}_{b} ( t ) ) , log ( - log {L}_{b} ( t ) ) ] ] = Pr [ β ∈ [ log \frac{log {U}_{b} ( t )}{log {S}_{0} ( t )} , log \frac{log {L}_{b} ( t )}{log {S}_{0} ( t )} ] ] = Φ ( \frac{log \frac{log {L}_{b} ( t )}{log {S}_{0} ( t )} - μ}{σ} ) - Φ ( \frac{log \frac{log {U}_{b} ( t )}{log {S}_{0} ( t )} - μ}{σ} ) .$$

The optimization problem was set up as follows:

$$arg {min}_{μ , σ} \frac{1}{\vert T \vert} {\sum }_{t ∈ T} \frac{1}{{σ}_{t}} {\vert Φ ( \frac{log \frac{log {L}_{b} ( t )}{log {S}_{0} ( t )} - μ}{σ} ) - Φ ( \frac{log \frac{log {U}_{b} ( t )}{log {S}_{0} ( t )} - μ}{σ} ) - 0 \text{.95} \vert}^{2} .$$

where σ(t) is the standard deviation of the target covariate's prior survival curve at time point t, and Φ(.) is the cumulative distribution function of the standard normal distribution. In the equation above, T is the time horizon in which the fitting is desired, noting that the interval range can affect the fitting quality. This problem is non-convex, and therefore instead of using gradient-based methods, a grid and find the “optimal” hyper-parameters is constructed. Once hyper-parameters are inferred for all the covariates, they will be used in the Bayesian Cox model described above.

To make this process clear, the results of parameter construction can be seen in FIGS. 8A & 8B, 18A & 18B, and 26A & 26B. In each case, the baseline survival function S₀(t) is shown in the first panel. The subsequent panels show both the underlying primary data (solid line+dashed line Cls) as well as S₀(t)^{exp(β)}—that is, the result of parameter optimization (~N(μ, σ²) for each given risk-factor. This shaded area is the 95% CI of the corresponding normal distribution of β. The μ and σ for each outcome predictor is shown as well.

### Parameter Calibration in CIRI for Survival Analysis

It is possible that different cohorts used for hyper-parameter inferences could have significantly different survival functions (compared to the baseline function) which can in turn affect the calibration to the observed risk. In order to address this issue, new coefficients were introduced, referred to as “calibrating coefficients” herein. These calibrating coefficients are found using the same approach described above for covariates, where σ(t), L_{b}(t) and U_{b}(t) are instead “cohort baseline function”, and the difference being the inferred mean (and ignore the variance) will only be employe. Once these means are inferred, they are subtracted from all the coefficients corresponding to the covariates whose prior knowledge came from that same cohort.

As an example, suppose that the prior knowledge for Stage and Grade in breast cancer from one cohort is found. First, the hyper-parameters is inferred for dummy variables defined based on Stage and Grade categories. Without loss of generality, for Stage=1, first construct the prior as β_{Stage1}~N(μ_{Stage1},

$${σ}_{Stage 1}^{2} )$$

and for Grade=1 as β_{Grade1}~N(μ_{Grade1},

$${σ}_{Grade 1}^{2} ) .$$

Next, by pooling all the patients in the same cohort (regardless of stage or grade), then σ(t), L_{b}(t) and U_{b}(t) is found. Then, using the optimization above, a calibrating factor, denoted by μ_{calibration} is found. In the example outlined above, the prior centers for Stage=1 and Grade=1, respectively, are adjusted as follows:

$${β}_{Stage 1} \sim N ( {μ}_{Stage 1} - {μ}_{calibration} , {σ}_{Stage 1}^{2} )$$

and

$${β}_{Grade 1} \sim N ( {μ}_{Grade 1} - {μ}_{calibration} , {σ}_{Grade 1}^{2} ) .$$

### Markov Chain Monte-Carlo Sampling in CIRI for Survival Analysis

In order to perform Markov Chain Monte Carlo (MCMC), Metropolis-Hastings algorithm was employed. The prior probability is defined as β~N(μ, Σ). In cases where there is no sample to update the prior, i.e. posterior is in fact the prior itself. However, one powerful characteristic of the proposed Bayesian model is the capability of updating the prior using a limited set of samples. In these scenarios one needs to add the likelihood function. There are two possibilities to add the likelihood function: (1) Cox partial likelihood function and (2) the exact likelihood function given the presumed baseline survival function. In order to make sure that the calibration is not affected the latter was chosen. Therefore, the log-likelihood function becomes

$${ℓ ( β \vert \{ {X}_{i} , {C}_{i} ) \}}_{i = 1 : n} ) = {\prod }_{i = 1} {\{ exp \{ {β}^{T} {Z}_{i} \} {λ}_{0} ( {T}_{i} ) \}}^{{Δ}_{i}} exp \{ - {\int }_{0}^{{T}_{i}} {λ}_{0} ( t ) dt \times {e}^{{β}^{T} {Z}_{i}} \}$$

where λ₀(.) Is the instantaneous hazard function, Δᵢ is the event indicator variable. Writing it in log-transformed form:

$${ℓ}_{l} = {\sum }_{i} [ {Δ}_{i} [ {β}^{T} {Z}_{i} + log {λ}_{0} ( {T}_{i} ) ] - {\int }_{0}^{{T}_{i}} {λ}_{0} ( t ) dt \times {e}^{{β}^{T} {Z}_{i}} ] .$$

### Now,

$${S}_{0} ( t ) = exp \{ - {\int }_{0}^{t} {λ}_{0} ( τ ) d τ \}$$

and therefore,

$$log {λ}_{0} ( t ) = log ( \frac{d}{dt} ( - log {S}_{0} ( t ) ) ) .$$

In this implementation, spline fitting was used to the prior knowledge survival Kaplan-Meier curves to estimate the instantaneous hazard function.

In the MCMC sampling, the proposal distribution was also tuned such that the acceptance rate falls in [0.2,0.3] interval. Whenever prior updating samples were available, a linear combination of prior mean and Cox proportional hazard model with ridge regression was used as the initial sample. 500 burnin samples were used in the MCMC with 10,000 samples in the actual predictions and 2,000 samples for the simulations comparing traditional Cox proportional hazard models to CIRI.

Estimation of Survival Functions from Published Literature

As outlined above, a number of model parameters were required to develop CIRI. These parameters were derived from previously published datasets or literature, depending on their availability. Specifically, these parameters were determined from survival data in each disease of interest. For DLBCL, event-free survival or EFS (either at 24 months for fixed-endpoint analysis or over time in survival analysis) was estimated; for CLL, progression-free survival or PFS (either at 36 months for fixed-endpoint analysis or over time in survival analysis) was estimated; for BRCA, distant-relapse free survival or DRFS (either at 36 months for fixed-endpoint analysis or over time in survival analysis) was estimated. To estimate these, information on survival was determined by one of three methods from established literature.
1.) When patient-level data was available from a prior study or data-set, this was preferentially used to determine survival functions by the Kaplan-Meier method. In this study, parameters for the following risk-factors were determined from patient-level data:
   a. CIRI-DLBCL—pretreatment ctDNA level, early molecular response, major molecular response
   b. CIRI-CLL—baseline survival function, CLL-IPI, interim MRD, final MRD
   c. CIRI-BRCA—baseline survival function, clinical stage, tumor grade, estrogen receptor/HER2 status.
2.) When patient-level data was not available, published Kaplan-Meier estimates of survival were used. Quantitative imaging analysis was performed on published survival curves to determine the survival function S(t), along with the number at risk (nᵢ), number of events (dᵢ), and probability of survival (pᵢ) during each time interval tᵢ (DataThief III, datathief.org); point estimates were manually reviewed against published survival outcomes to confirm accuracy. The probability of survival at the time of interest was then determined directly from the function S(t). When multiple survival estimates needed to be combined (either to combine multiple groups or to combine multiple literature sources), these were combined as follows:

Given two Kaplan-Meier estimates of survival, S₁(t) and S₂(t) with equally spaced time intervals, the number at risk and number of events for the combined survival curve S_{combined}(t) for a given time interval tᵢ are:

$${n}_{i , combined} = {n}_{i , 1} + {n}_{i , 2}$$

$${d}_{i , combined} = {d}_{i , 1} + {d}_{i , 2}$$

The probability of survival during the interval tᵢ, termed p_{i,combined}, is therefore:

$${p}_{i , combined} = ( {n}_{i , combined} - {d}_{i , combined} ) / {n}_{i , combined}$$

In this study, parameters for the following risk-factors were determined from previously published Kaplan-Meier survival curves:
a. CIRI-DLBCL—baseline survival function, IPI, and cell of origin
b. CIRI-CLL—N/A
c. CIRI-BRCA—residual cancer burden
3.) When neither patient-level data nor Kaplan-Meier estimate of the survival function were available, estimates of the probability of survival at fixed points in time were used. In this study, parameters for the following risk-factors were determined from survival at fixed time-points:
a. CIRI-DLBCL—interim imaging
b. CIRI-CLL—N/A
c. CIRI-BRCA—N/A.

The above methods were used to determine the expectation value for each survival function. The variance, standard deviation, and 95% confidence intervals for survival were then estimated using Greenwood's formula. These confidence intervals were used for parameter estimation in the CIRI model for survival analysis as described above.

### Simulations to Assess the Effect of Correlated Coefficients on CIRI

In order to evaluate the effect of correlation between CIRI coefficients (Bs), a series of models were simulated with different degrees of correlation between coefficients. To generate synthetic models and draw samples from that model, the following steps were performed (using the part of the data sets which was used for prior construction): (1) for 1,000 bootstrap resampling of the full data, the Cox proportional hazard model was solved (i.e. inferred the corresponding regression coefficients), (2) a covariance matrix using these 1,000 random realizations was built, (3) the mean of the β vector was calculated, (4) the diagonal elements of the matrix were fixed (i.e. individual variances), the direction of the off-diagonal elements was kept (i.e. positive or negative correlation), the off-diagonal elements were changed to the desired value, I.e.

$${σ}_{ij}^{new} = sign ( {σ}_{ij}^{old} ) * \vert ρ \vert * {σ}_{i}^{old} {σ}_{j}^{old} ,$$

and denoted that by Σₚ, (5) samples from a multivariate normal distribution were randomly generated with the mean and covariance matrix generated in (3) and (4), respectively. Associated with each β, the original covariate matrix (matrix X) was utilized, the β^{T}X was calculated, and via the baseline and Cox proportional hazard model, random time of events was generated. The same rate of censoring time was also generated by sampling from the censor value in the original data set. Note that step (4) might lead to negative definite matrix, where in those situations 1.01×|λₘᵢₙ|×I was added to the covariance matrix Σ_{ρ} where λₘᵢₙ (<0) is the minimum eigenvalue of Σ_{ρ}.
Comparison with Cox Proportional Hazard Model

CIRI was compared with the standard Cox proportional hazard model. A simulation setup was implemented for breast cancer and chronic lymphocytic leukemia, the two diseases for which there was a large number of samples. Three metrics for performance evaluation were considered: (1) area under receiver operating characteristic (ROC) curve or C-statistic, (2) the calibration-in-the-large intercept defined as

$$\vert \frac{1}{{n}_{test}} {\sum }_{i} {Pr}^{CIRI} ( Surv \vert indiv . \# i ) - {Pr}^{KM} ( Surv \vert full cohort ) \vert ,$$

and (3) the calibration slope. For the simulation setup nₜₑₛₜ=150 for breast cancer and 400 for CLL, were used; however, the number of training samples were varied: nₜᵣₐᵢₙ∈{20, 40, . . . , 200}. For M=250 iterations, the datasets were split into test set Sₙ_{ₜₑₛₜ} and train Sₙ_{ₜᵣₐᵢₙ}. The training samples Sₙ_{ₜᵣₐᵢₙ} were used for inferring Cox model coefficients, and also updating CIRI framework via MCMC. Both models were then applied to the test set Sₙ_{ₜₑₛₜ} and calculated the three metrics above. Metric #1 was calculated using R package survivalROC (version 1.0.3) at T=36 (months). In order to calculate metrics #2 and 3 a baseline function is required, which is inherently part of the CIRI framework, however it is not so for the Cox model. Therefore, the cumulative hazard function, H (t), was further estimated after estimating the Cox coefficients (still using the training data) and was plugged in to obtain the corresponding baseline function S₀(t)=exp {−H(t)}.

### Prediction of Therapeutic Benefit in Subsets of Patients

To explore the possibility of using CIRI to discover predictive biomarkers—i.e., patient subsets defined quantitatively by CIRI who preferentially benefit from specific therapies—proof-of-concept CIRI models were constructed agnostic to therapy selection, integrating pretreatment and interim predictors. For example, in the case of CLL, a CIRI model was constructed integrating only the CLL-IPI and interim MRD, that was not informed by choice of initial therapy. Predictions were then made for the probability of outcome (PFS at 36 months) for each patient using only this data. Furthermore, a “prediction” of the likely effect of each therapy on each patient was also made—i.e., what the likely benefit of every possible therapy would be for each individual. This involved making a personalized outcome prediction for each patient for each possible immunochemotherapy (FCR, BR, R-chlorambucil, G-chlorambucil). In each case, the predicted benefit from treatment for groups of patients defined by various CIRI thresholds was compared with actual outcomes (e.g., in CLL), and assessed for specific thresholds of CIRI risk that could identify a selective therapeutic benefit in a patient subpopulation (e.g., in CLL). 95% confidence intervals for the predicted benefit of treatment (FIG. 5A) was determined from 10,000 bootstrap resamplings of patients in each risk group; 95% confidence intervals for the observed benefit of treatment was obtained from Greenwood's formula.

Existing literature does not well capture the full set of parameters relating to each individual therapy or therapeutic combination. Therefore, the data-driven approach was adapted to deriving priors as used in CIRI to identify parameters related to therapeutic selection. In the case of CLL, this consisted of the therapies used in the CLL8, CLL10, and CLL11 dataset; for breast cancer, this consisted of neoadjuvant chemotherapy+/−Trastuzumab as per the ISPY trial (L. J. Esserman, et al., (2012), cited supra), or neoadjuvant chemotherapy+Trastuzumab+Pertuzumab as per the TRYPHAENA trial (M. Ignatiadis, et al., (2019), cited supra). The previously established CIRI prognostic model priors was used for other shared covariates. Since the disease subsets within the existing cohorts with available prognostic covariates, treatment assignment, and outcome data are often small, to construct robust priors for treatment and other new covariates, a k-fold cross-validation (k−CV) framework was implemented. In each iteration of this CV framework, (k−1) folds of patients were used to derive maximum-likelihood estimates of the survival probability (i.e. Kaplan-Meier curves) along with their 95% confidence bands. Next, the CIRI prognostic model was used to infer the hyper-parameters from these estimates. The final CIRI model built using these priors was then applied to the remaining “held out” patients in each fold to predict the outcome. In the case of CLL, this cross-validation framework was applied to patients from the CLL8, CLL10, and CLL11 trials (B. Eichhorst, et al., (2016), cited supra; V. Goede, et al., (2014), cited supra; and M. Hallek, et al., (2010), cited supra), limiting the analysis to only patients with interim MRD assessment receiving immuno-chemotherapy. For breast cancer, patients were pooled from two clinical trials where neoadjuvant therapy was given (ISPY and TRYPHAENA) and where data was publicly available (L. J. Esserman, et al., (2012), cited supra; and M. Ignatiadis, et al., (2019), cited supra). Here, the analysis was limited to HER2+ patients where survival data was available. In the ISPY dataset, RCB scores were available. In the TRYPHAENA dataset, only pathologic CR (pCR) status was available; therefore, for the purpose of the CIRI model, patients achieving a pCR were assigned an RCB score of 0, while patients not achieving pCR were assigned an RCB score of 3.

To assess the ability of CIRI to identify a subgroup of patients who preferentially benefit from a given therapy (i.e., act as a “predictive” biomarker), a region [symbol] should be found in which the treatment effect is significantly better than the average treatment effect. This subgroup is traditionally defined heuristically by constraining the covariates, e.g. [symbol] ={x₁<a, x₂>b}. Here, the prognostic model CIRI was employed to find this subgroup. Denoting CIRI prediction for covariate vector X at time t₀ by S_{CIRI} (t₀|X), e.g. t₀=36mo for CLL, a CIRI based subgroup was defined as [symbol] ₛ_{₀}={X∈X|S_{CIRI} (T₀|X)>s₀}, where X is the entire covariate space. Two treatment benefit variables was then defined as:

$${Δ}_{CIRI} ( {𝒫}_{{s}_{0}} ) = \text{?} ( Y = 1 \vert T = 1 , X ∈ {𝒫}_{{s}_{0}} ) - \text{?} ( Y = 1 \vert T = 0 , X ∈ {𝒫}_{{s}_{0}} ) ,$$

$${Δ}_{CIRI} ( 𝒳 \backslash {𝒫}_{{s}_{0}} ) = \text{?} ( Y = 1 \vert T = 1 , X ∉ {𝒫}_{{s}_{0}} ) - \text{?} ( Y = 1 \vert T = 0 , X ∉ {𝒫}_{{s}_{0}} )$$

which respectively quantify the benefit of the patients whose covariates satisfy [symbol] ₛ_{₀}, and patients who do not. In these equations, \hat{P}r(.) denotes the CIRI predictions, and Y denotes the binary outcome. The predictive threshold set as all CIRI thresholds leading to subgroup treatment benefit was defined as follows

$${}^{\text{?}} = \{ {s}_{0} \vert {Δ}_{CIRI} ( {𝒫}_{{s}_{0}} ) {>}^{*} 0 and {Δ}_{CIRI} ( 𝒳 \backslash {𝒫}_{{s}_{0}} ) {<}^{*} 0 \}$$

where >* and <*, denote greater and less than in probability (i.e. evaluated by significance p-values), respectively.
Framework for Therapeutic Selection from CIRI Risk Estimates

In addition to identifying patients at high-risk of disease progression and associated mortality, prognostic biomarkers should ideally help make therapeutic decisions to overcome such risk. For example, previous studies have attempted to identify DLBCL patients for early treatment intensification using either the IPI or interim PET/CT scans. Unfortunately, these approaches have largely failed to improve survival.

Quantitative risk assessment with CIRI-DLBCL can lend insight into these results. A decision to change therapy could be considered as a test of two competing therapeutic strategies. In the case of DLBCL, alternative options are 1) to continue and complete frontline therapy, or 2) transition to a salvage regimen, typically high-dose chemotherapy followed by autologous hematopoietic stem cell transplantation (ASCT) (M. Herzberg, et al., Haematologica 102, 356-363 (2017); and P. J. Stiff, et al., The New England journal of medicine 369, 1681-1690 (2013); the disclosures of which are each incorporated herein by reference). A statistical test between the probability of favorable outcome (i.e., event-free survival at 24 months) with each of these options could help identify the ideal treatment course. A quantitative distribution of likely outcomes with frontline therapy for a given patient can be obtained through the Bayesian analysis framework within CIRI. A conservative estimate of the probability of favorable outcome with salvage ASCT can be obtained from prior studies in the salvage setting (M. Crump, et al., Journal of clinical oncology: official journal of the American Society of Clinical Oncology 32, 3490-3496 (201), the disclosure of which is incorporated herein by reference).

To explore the predicted benefit from early ASCT in patients with high IPI or positive interim PET/CT scans, two typical patients were considered with these single risk-factors and their predicted probability of achieving EFS24 after frontline therapy (FIG. 45). These two risk profiles were compared to the average outcome probability for patients receiving salvage therapy and ASCT in the second line (grey). Remarkably, patients whose only unfavorable features relate to baseline clinical risk or poor radiographic responses are, on average, unlikely to benefit from such a change in therapy, as their predicted outcomes after first-line therapy are superior to the average patient receiving subsequent ASCT.

Risk estimates from CIRI-DLBCL were also considered to identify individual patients likely to benefit from an early intervention with ASCT. Unlike with interim radiographic evaluation alone, CIRI identified individual patients where the predicted risk after first-like therapy that was inferior to the average outcome with second-line therapy (e.g., patient DLBCL103 in FIG. 46). By comparing the personalized predicted outcome after traditional frontline therapy versus ASCT for each patient over time, CIRI was used to estimate the statistical likelihood of the benefit of a change in therapy at this milestone.

To perform this statistical test, the personalized probability of EFS24 was compared with frontline therapy to the probability of EFS24 for the average patient with salvage therapy—namely, autologous stem cell transplantation (ASCT)—determined from the LY.12 trial, established in prior literature (C. Crump, et al., (2014), cited supra) (this probability was determined as described in the “Estimation of survival functions from published literature” section). To evaluate the probability that an individual patient would benefit from a switch in therapy, the personalized value was calculated:

$$P ( EFS {24}_{RCHOP} ) - P ( EFS {24}_{salvage} )$$

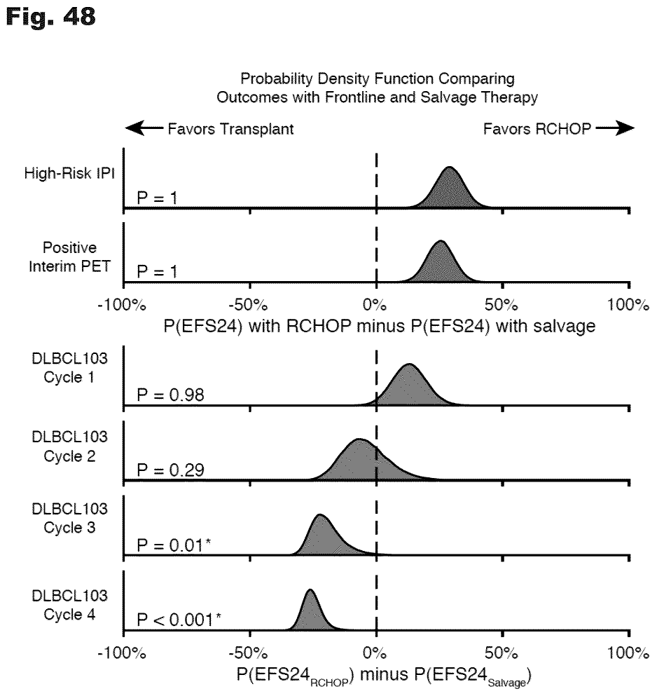

which represents the difference in likely outcomes with RCHOP therapy vs salvage ASCT. The distribution of this posterior probability was calculated by 10,000 Markov Chain Monte Carlo samplings. This process is shown for an individual patient, DLBCL103, resulting in the probability density functions shown in FIG. 48. empiric P-values was then calculated for the probability that:

$$P ( EFS {24}_{RCHOP} ) - P ( EFS {24}_{salvage} ) < 0$$

This P-value represents the probability that a switch in treatment—from RCHOP to salvage therapy-would represent a superior treatment option for patient DLBCL103.

### Determination of Model Calibration

For any predictive model, determination of correct calibration is essential—that is, if a patient is predicted to have a 25% risk of an event within 24 months of diagnosis, this patient should truly have a 25% risk. If the true risk is significantly higher or lower than this, the model is said to be poorly calibrated. As CIRI provides an updating risk-estimate for each patient as more information is obtained throughout a course of treatment, it was sought to ensure that CIRI was calibrated at the time of each prediction—for example, in the case of CIRI-DLBCL, where a prediction is made for each patient at four times (before and after 1, 2, or 3 cycles of therapy), a total of 528 predictions across 132 patients were evaluated for calibration.

For CIRI-DLBCL, CIRI-CLL, and CIRI-BRCA, calibration-in-the-large, or the difference between the predicted and observed risk of an event (Pr(event, Observed) vs Pr(event, Predicted)), was assessed. In each case, the predicted Pr(event) was calculated as the mean predicted risk at an endpoint of interest. For example, in the fixed-endpoint formulation of CIRI, the observed and expected probability of an event by 24 months (Pr_{obs}−Prₕₐₜ) was compared. To account for censoring, the observed Pr(event) was calculated by the Kaplan-Meier method. These probabilities and their difference are provided along with each calibration plot for each model. The 95% confidence interval for this assessment of calibration-in-the-large is also provided (via 2000 bootstraps).

Next, model calibration was assessed through calibration plots. Here, predictions were divided into deciles of estimated risk. Similar to above, the mean predicted risk was compared to the observed risk (calculated by Kaplan-Meier method) and is shown on the plots. Calibration was initially assessed visually, where each risk quantile should remain close to the line x=y (dashed line on each plot). Patient-level outcome data is also shown, plotting the predicted risk versus a moving-average smoothed observed risk for each patient. Both the patient-level and quantile-level data were visually calibrated.

Calibration can be assessed quantitatively via calibration plot by performing linear regression of the predicted vs. observed risk. Here, the intercept, given a slope of 1 (termed the ‘Calibration Intercept’ in this paper), provides another estimate of calibration-in-the-large (intercept should be 0 in perfect calibration). This metric and 95% confidence interval is provided in each calibration plot. Additionally, the slope of this linear regression (i.e., ‘Calibration Slope’) provides another assessment of calibration, where a slope of 1 represents perfect calibration. The Calibration Slope and 95% confidence intervals are also provided with each calibration plot.

### Assessment of CIRI Performance by C-Statistic

In addition to the calibration of a prediction model, the predictive value of the model is also essential (i.e., the discrimination power of the model). The various models' performance were assessed using the area under receiver operator characteristic curve, or C-statistic. Given that survival data includes possible censorship, the C-statistic was calculated accounting for censored data as per Heagerty et al. (P. J. Heagerty, T. Lumley, and M. S. Pepe, Biometrics 56, 337-344 (2000), the disclosure of which is incorporated herein by reference). This requires selection of a time-point of interest; in the case of CIRI-DLBCL for fixed-endpoint, this was EFS and OS at 24 months (FIGS. 7A & 7B). In the CIRI-DLBCL model for survival analysis, this was EFS and OS from 12 to 36 months (FIGS. 12 & 14); for the CIRI-CLL model for survival analysis, this was PFS and OS from 12 to 60 months (FIGS. 21 & 23); finally, for the CIRI-BRCA for survival analysis, this was DRFS from 12 to 60 months (FIG. 30). Confidence intervals and empiric P-values for comparison with each individual risk stratification tool were performed from 2000 bootstrap resamplings.

### Additional Statistical Analyses

Survival probabilities were estimated using the Kaplan-Meier method; survival of groups of patients base on ctDNA levels were compared using the log-rank test. All analyses were performed with the use of MATLAB, version 2017a, R Statistical Software version 3.4.1, and GraphPad Prism, version 7.0a. Calculation of AUC accounting for censorship was performed using the R ‘survivalROC’ package version 1.0.3 with default settings.

### Example 2: Applying Continuous Individualized Risk Index to Non-Small Cell Lung Cancer (CIRI-NSCLC)

Circulating tumor DNA (ctDNA) molecular residual disease is highly prognostic for disease progression for non-small cell lung cancer (NSCLC), but there are currently no effective methods to monitor response to treatment, including chemoradiation treatment (CRT), to enable response-adapted therapies. Integrating pre-CRT tumor features with mid-CRT ctDNA analysis, a Continuous Individualized Risk Index (CIRI-NSCLC) was developed and validated, which accurately predicts progression-free survival (PFS) in patients with NSCLC undergoing CRT. CIRI-NSCLC during CRT performs comparably to ctDNA molecular residual disease analysis after completion of therapy. These results suggest that combining pre-CRT risk factors with ctDNA molecular residual disease analysis can identify patients at very high and low risk of progression to enable response-adapted therapies.

### Biological and Molecular Prognostic Factors in Locoregionally Advanced NSCLC

It was hypothesized that combining mid-CRT ctDNA analysis with additional prognostic factors could reduce false negatives and improve prediction of progressive disease. However, few prognostic factors have been validated in patients treated with CRT for NSCLC. Therefore, a separate historical cohort of 108 patients with stage IIB-IIIA NSCLC from Stanford University and the Cancer Genome Atlas (TCGA) treated with radiation therapy was established to identify and train additional features prognostic of PFS.

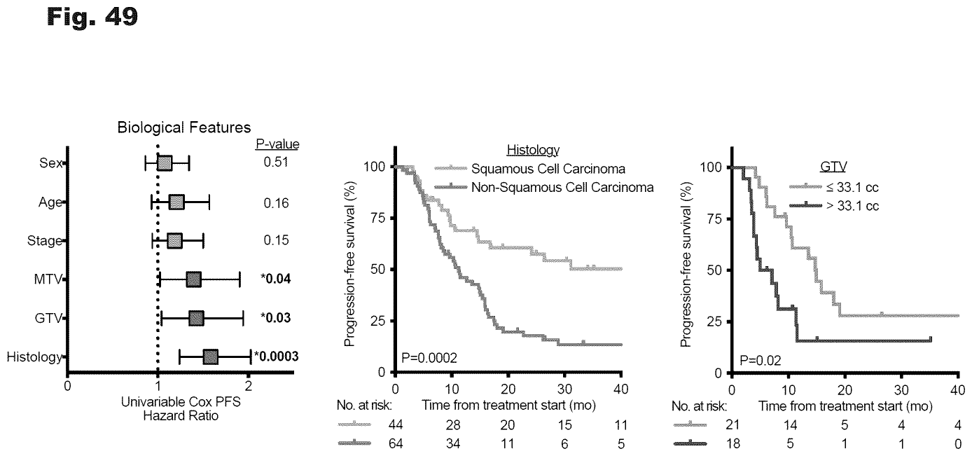

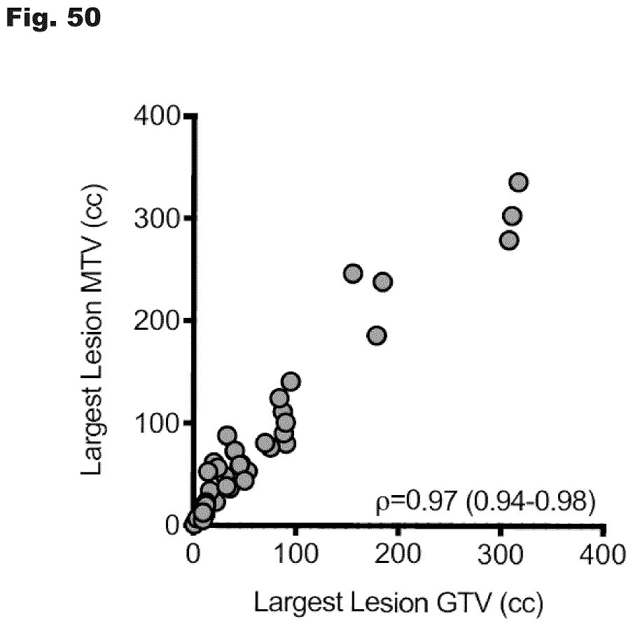

Various prognostic factors were assessed for NSCLC treated with CRT as were predictors of local recurrence. Within the historical training cohort, pre-CRT largest lesion metabolic tumor volume (MTV), largest lesion gross tumor volume (GTV), and histology (non-squamous cell carcinoma vs. squamous cell carcinoma) were significantly associated with PFS (FIG. 49). However, there was not a significant association of sex, age, or stage with PFS. Further, GTV and MTV were highly correlated (FIG. 50).

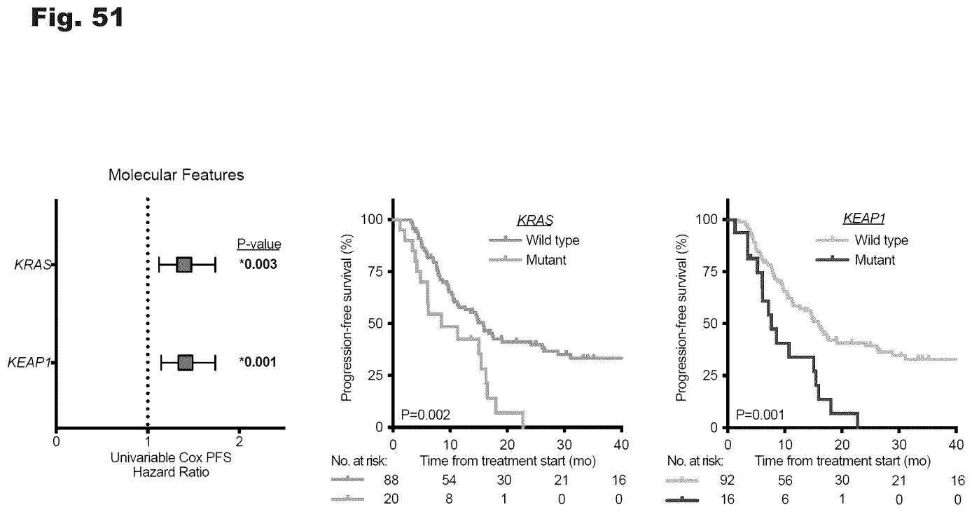

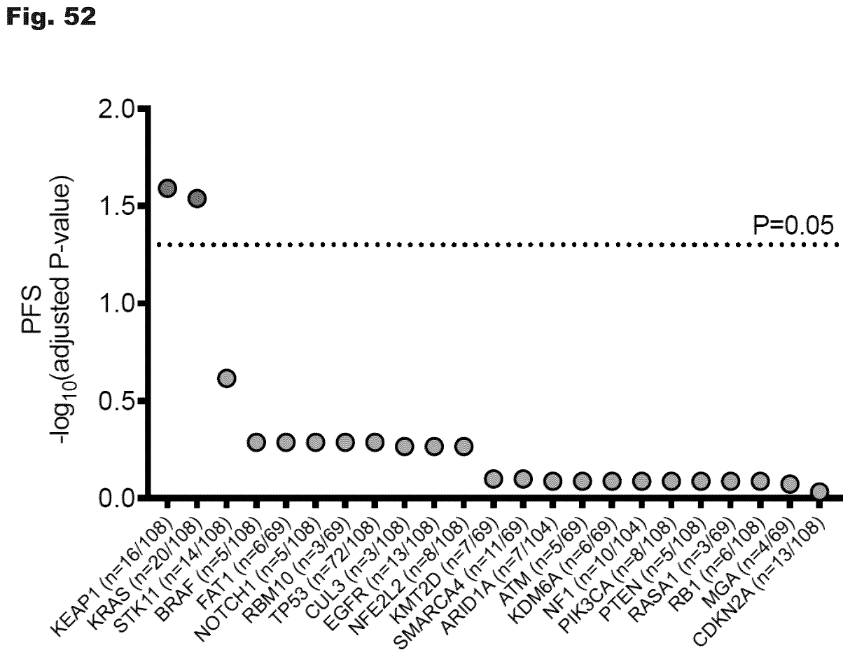

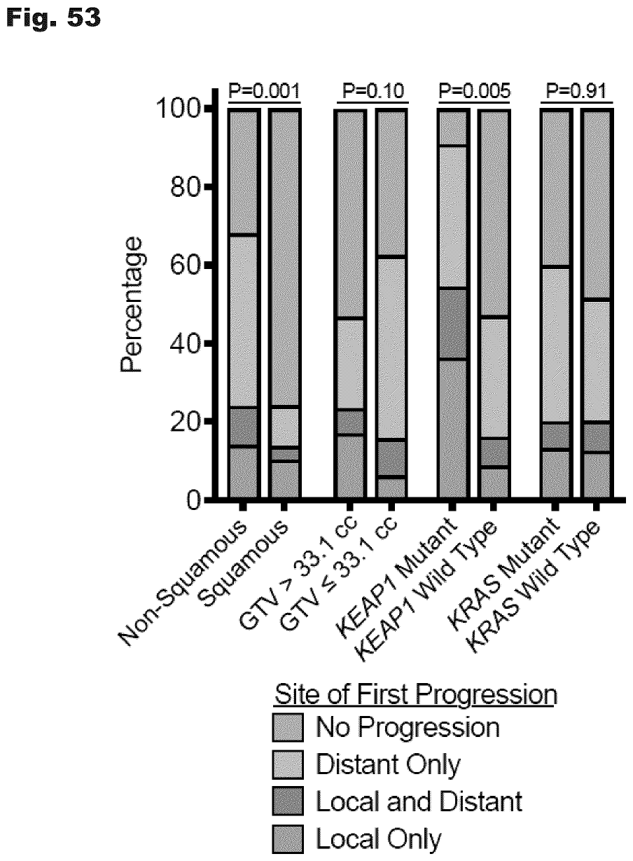

Although mutations in KRAS and TP53 have been associated with poor outcomes in unselected cohorts of NSCLC, very few studies have exclusively examined locoregionally advanced NSCLC treated with CRT. To identify molecular features associated with PFS in locoregionally advanced NSCLC, previously identified lung cancer driver genes observed in at least 5% of lung adenocarcinomas or lung squamous cell carcinomas were analyzed. Of the 23 genes analyzed, only KRAS and KEAP1 mutations were significantly associated with PFS (FIGS. 51 & 52). Non-squamous cell carcinomas more frequently failed distantly (FIG. 53). In addition, tumors with KEAP1 mutations more frequently failed within the radiation field. However, there was no difference in pattern of recurrence in patients with larger GTVs or KRAS mutant tumors.

### A Dynamic Risk Index for NSCLC

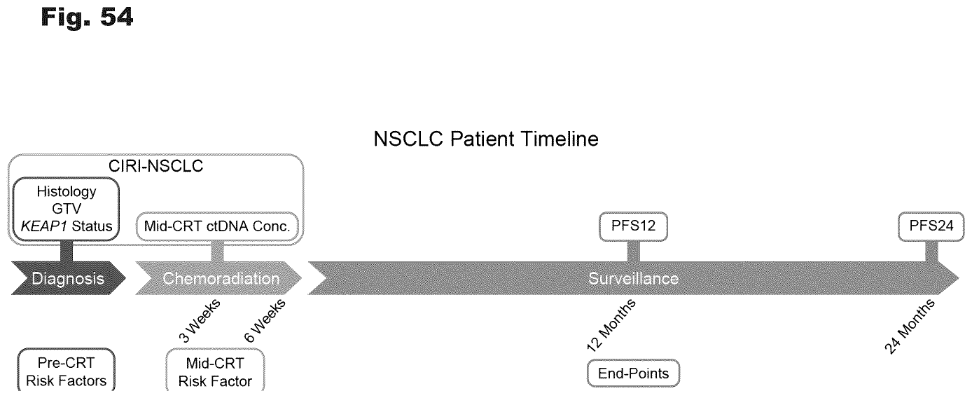

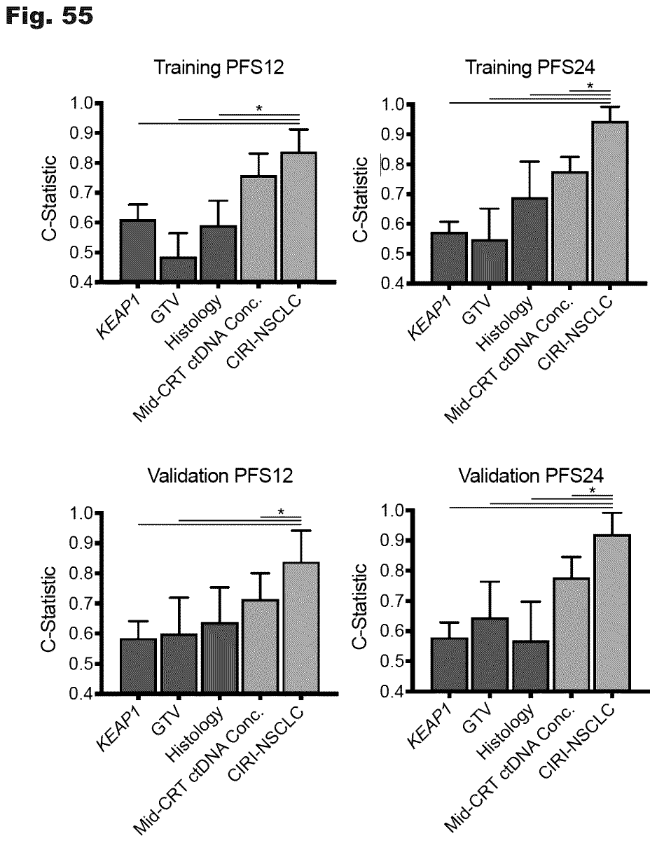

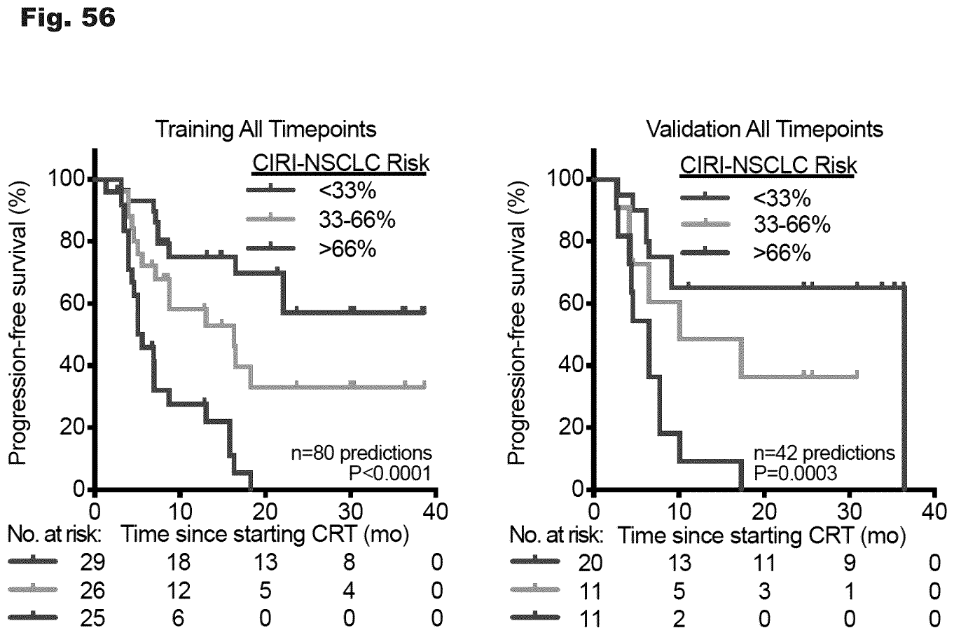

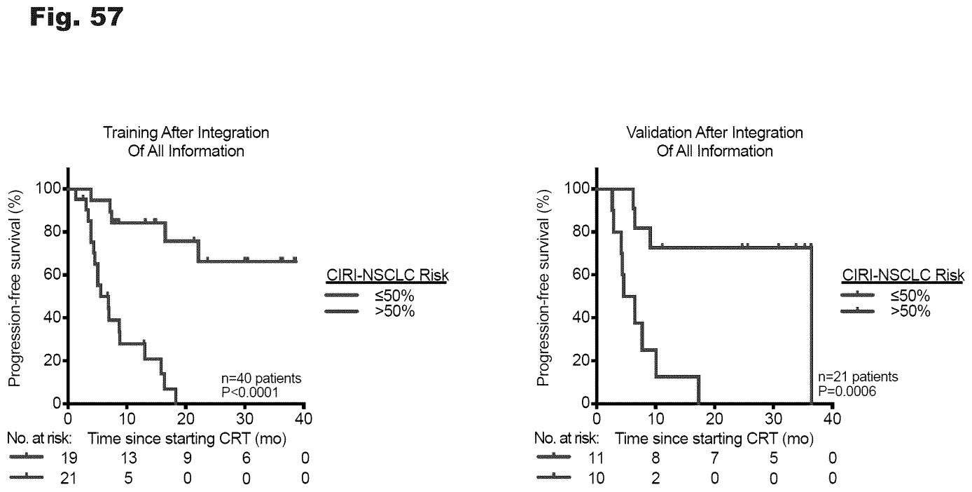

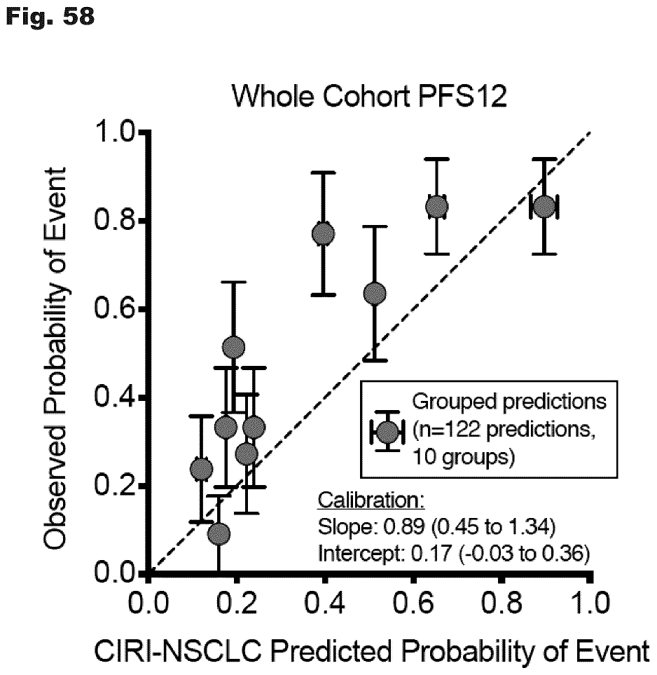

Having identified pre-CRT prognostic factors for PFS, these factors were combined with mid-CRT ctDNA changes to improve prediction of progressive disease during CRT for NSCLC. A prognostic model for locoregionally advanced NSCLC treated with CRT was built that incorporated pre-CRT and mid-CRT risk factors called CIRI-NSCLC (FIG. 54). All possible combinations of significant biological, molecular, and ctDNA features were evaluated. Because of the strong correlation between GTV and MTV along with the slightly stronger association of GTV with PFS, only GTV was included in the model. CIRI-NSCLC performed best in the training cohort when incorporating KEAP1 mutation status, histology, largest lesion GTV, and mid-CRT ctDNA concentration. This CIRI-NSCLC model improved prediction of PFS at 12 and 24 months by C-statistic, significantly outperforming individual risk factors including mid-CRT ctDNA concentration in the training and validation cohorts (FIG. 55). Furthermore, patients could be stratified into risk groups by aggregating all CIRI-NSCLC predictions of progression or death by 24 months or by considering the full CIRI-NSCLC model (FIGS. 56 & 57). Good calibration of the model was observed across the whole cohort when comparing predicted and observed risk of PFS at 12 months (FIG. 58).

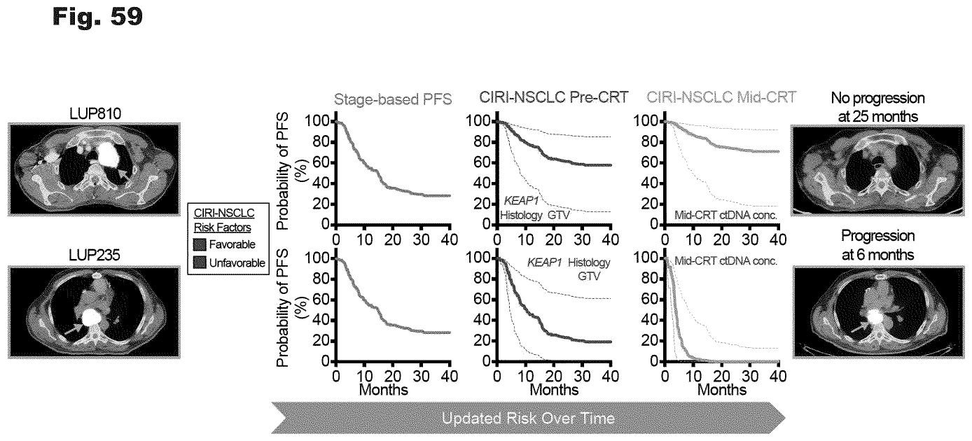

CIRI-NSCLC enabled individualized real-time updating of the probability of PFS as model features became available over the course of CRT. For example, two patients in the validation cohort, LUP810 and LUP235, were both treated with CRT for Stage IIIA NSCLC (FIG. 59). LUP810 presented with a left upper lobe squamous cell carcinoma with a GTV of 60.3 cc and wild type KEAP1, corresponding to a 38% CIRI-NSCLC pre-CRT risk of progression or death at 24 months. Mid-CRT, the patient's ctDNA concentration was 1.7 hGE/ml, lowering his CIRI-NSCLC risk to 25%. Now 25 months after starting CRT, LUP810 remains disease-free. In contrast, LUP235 presented with a central adenocarcinoma with a GTV of 79.9 cc and wild type KEAP1, leading to a 76% CIRI-NSCLC pre-CRT risk. At his mid-CRT blood draw, his ctDNA concentration was 37.8 hGE/ml corresponding to a 100% CIRI-NSCLC risk of progression. He ultimately developed a local recurrence and distant brain metastases 6 months after starting CRT. These results show that CIRI-NSCLC improves prediction of PFS, enabling accurate risk stratification during CRT for NSCLC.

Comparing CIRI-NSCLC with ctDNA Molecular Residual Disease

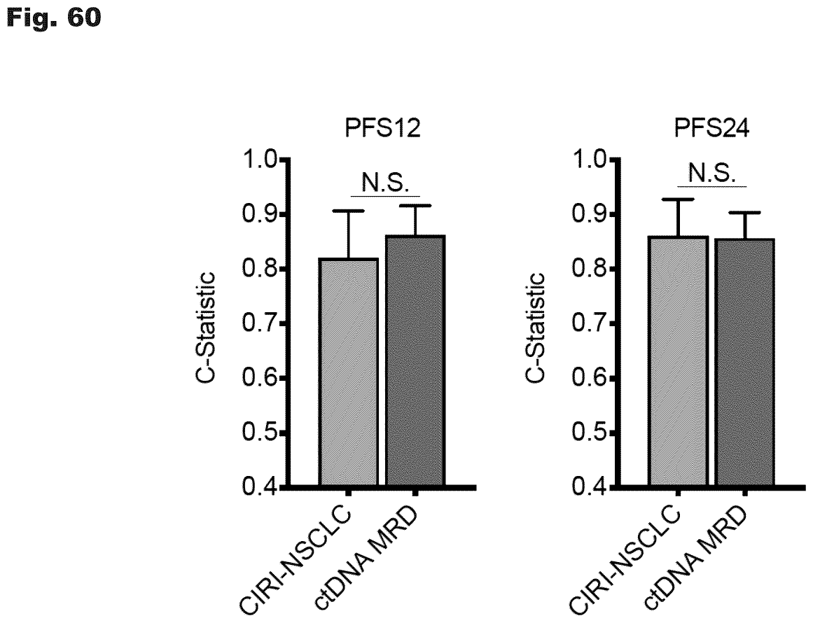

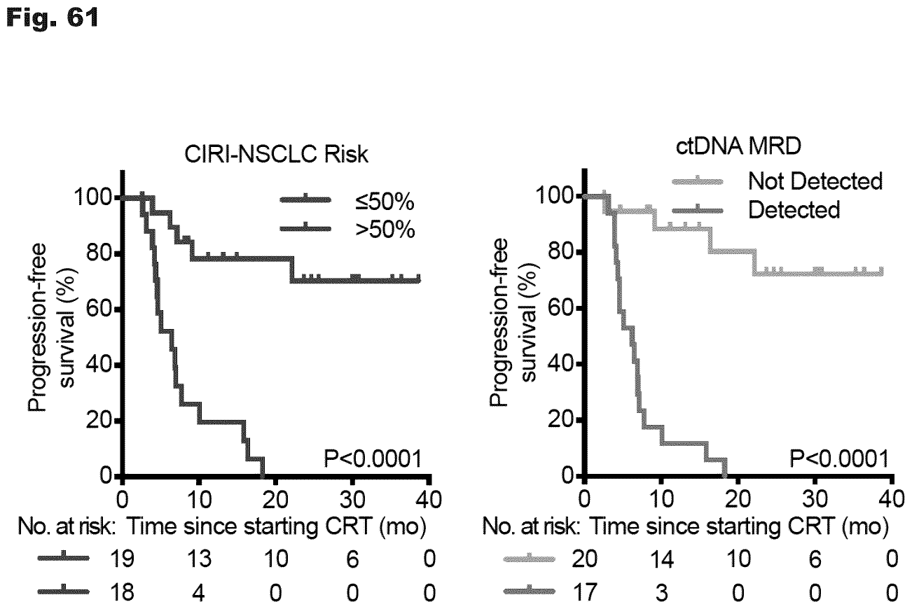

Given the excellent performance of CIRI-NSCLC for predicting PFS during CRT for NSCLC, CIRI-NSCLC was compare with detection of ctDNA molecular residual disease after completion of treatment. 37 patients across the training and validation cohorts were identified with plasma samples available for analysis from the first follow up visit after completion of all chemotherapy and radiation. Despite the mid-CRT plasma sample being collected a median of 2.1 months prior to the molecular residual disease plasma sample, CIRI-NSCLC performed comparably to ctDNA molecular residual disease for prediction of PFS at 12 and 24 months by C-statistic (FIG. 60) and Kaplan-Meier analysis (FIG. 61). In patients who ultimately progressed or died who were correctly predicted by both approaches, CIRI-NSCLC provided a 2.7 month median improvement in lead time over ctDNA molecular residual disease.

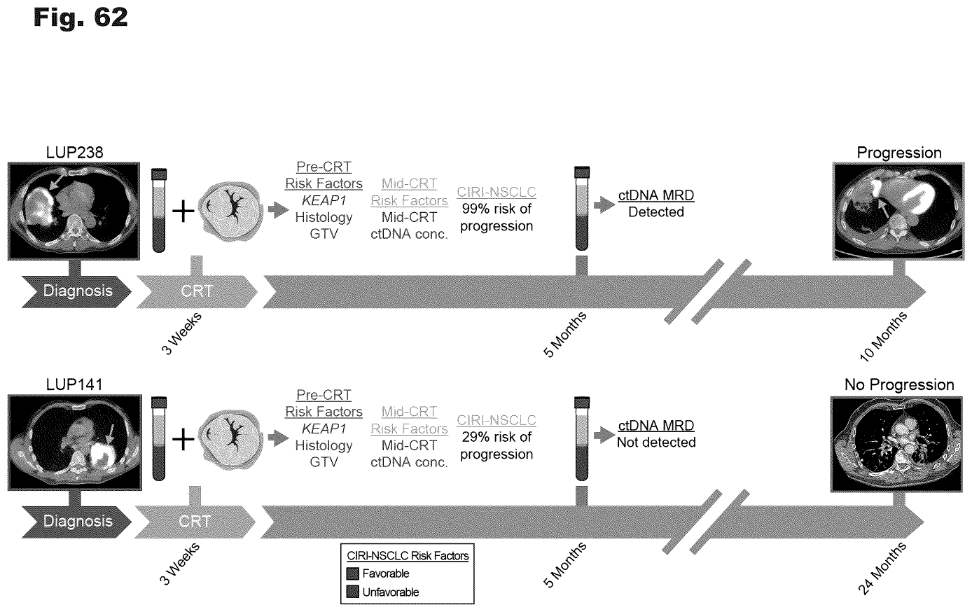

Two patients illustrate the ability of CIRI-NSCLC to provide an earlier predictor of PFS than ctDNA molecular residual disease (FIG. 62). LUP238 underwent CRT for a stage IIIA right middle lobe squamous cell carcinoma and ultimately developed local and distant disease progression 10 months after starting treatment. Four months prior to having ctDNA molecular residual disease detected, he had a 99% CIRI-NSCLC risk of progression or death by 24 months base on pre-CRT risk factors and mid-CRT ctDNA analysis. In contrast, LUP141 remained alive and progression free 24 months after completing CRT for a stage IIB squamous cell carcinoma of the left lower lobe. His mid-CRT CIRI-NSCLC risk of progression or death by 24 months was 29%, and ctDNA molecular residual disease was not detected 4 months later after completing treatment. Overall, these findings illustrate the potential for CIRI-NSCLC to provide a substantially earlier prediction of disease progression or death over ctDNA molecular residual disease, enabling earlier treatment escalation or de-intensification based on pre-CRT prognostic factors and mid-CRT ctDNA analysis.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

1. (canceled)

2. A method of treating an individual suffering from a disorder, comprising administering a treatment to the individual having a medical disorder assessment, wherein the medical disorder assessment comprises a plurality of sets of clinical data obtained throughout a course of the disorder, wherein the medical disorder assessment comprises a Bayesian framework assessment based on:
(i) the plurality of sets of clinical data comprising a plurality of outcome predictors, and
(ii) a set of prior knowledge of a performance of each of the plurality of outcome predictors as established from a) prior literature or b) the plurality of sets of clinical data; and
wherein the plurality of sets of clinical data comprises an initial set of clinical data of the individual obtained prior to an initial treatment of the individual and a subsequent set of clinical data obtained after the initial treatment.

3. The method of claim 2, further comprising administering a subsequent treatment to the individual based on an additional subsequent set of clinical data of the individual and a subsequent medical disorder assessment of the individual's initial, subsequent, and additional subsequent sets of clinical data.

4. The method of claim 2, wherein the disorder is a cancer.

5. The method of claim 4, wherein the cancer is selected from the group consisting of diffuse large B-cell lymphoma (DLBCL), chronic lymphocytic leukemia (CLL), and breast adenocarcinoma (BRCA).

6. The method of claim 4, wherein the cancer is:
a) diffuse large B-cell lymphoma (DLBCL) and the initial set of clinical data includes at least one of: international prognostic index, molecular cell of origin, quantity of initial circulating tumor DNA, or a medical image scan;
b) chronic lymphocytic leukemia (CLL) and the initial set of clinical data comprises results of first line of therapy or international prognostic index;
c) breast adenocarcinoma (BRCA) and the initial set of clinical data comprises clinical stage, tumor grade, or status of estrogen receptor (ER) and human epidermal growth factor receptor 2 (HER2); or
d) non-small cell lung cancer (NSCLC) and the initial set of clinical data comprises gross tumor volume, KEAP1 mutational status, or histology.

7. The method of claim 4, wherein the cancer is:
a) diffuse large B-cell lymphoma (DLBCL) and the subsequent set of clinical data comprises quantity of circulating tumor DNA (ctDNA) or a medical image scan;
b) chronic lymphocytic leukemia (CLL) and the subsequent set of clinical data comprises minimal residual disease;
c) breast adenocarcinoma (BRCA) and the subsequent set of clinical data comprises pathological response to therapy; or
d) non-small cell lung cancer (NSCLC) and the subsequent set of clinical data comprises ctDNA molecular residual disease.

8. The method of claim 4, wherein the cancer is:
a) diffuse large B-cell lymphoma (DLBCL) and the medical disorder assessment indicates event free survival;
b) diffuse large B-cell lymphoma (DLBCL) and the medical disorder assessment indicates overall survival;
c) chronic lymphocytic leukemia (CLL) and the medical disorder assessment indicates progression free survival;
d) breast adenocarcinoma (BRCA) and the medical disorder assessment indicates distant relapse free survival; or
e) non-small cell lung cancer (NSCLC) and the medical disorder assessment indicates progression free survival.

9. The method of claim 2, wherein the disorder is a cancer, and the initial set and the subsequent set of clinical data comprise a quantity of circulating tumor DNA.

10. The method of claim 2, wherein the treatment is different from the initial treatment based on a risk estimate from the medical disorder assessment, and wherein the risk estimate indicates that the initial treatment is not improving the disorder.

11. The method of claim 2, wherein the disorder is a cancer; wherein the initial set of clinical data comprises international prognostic index, molecular cell of origin, and quantity of initial circulating tumor DNA; and wherein the subsequent set of clinical data comprises a quantity of circulating tumor DNA, minimal residual disease, pathological response to treatment, or a medical image scan.

12. The method of claim 2, wherein the disorder is a cancer; wherein the initial set of clinical data comprises international prognostic index, molecular cell of origin, and quantity of initial circulating tumor DNA; and wherein the subsequent set of clinical data comprises a quantity of circulating tumor DNA, minimal residual disease, pathological response to treatment, and a medical image scan.

13. The method of claim 3, wherein additional subsequent sets of clinical data comprise a quantity of circulating tumor DNA, minimal residual disease, pathological response to treatment, and/or a medical image scan.

14. The method of claim 2, wherein the Bayesian framework assessment comprises a Bayesian proportional hazard model to determine the continuous survival function over time for the individual.

15. The method of claim 2, wherein the Bayesian framework assessment comprises a naïve Bayesian model that predicts outcomes at a fixed endpoint.

16. The method of claim 2, wherein the Bayesian framework assessment is repeatedly updated by new sets of clinical data.

17. The method of claim 2, wherein the disorder is a cancer, and wherein the treatment is a chemotherapy or an immunotherapy.

18. The method of claim 2, wherein the treatment is based on predicting a pathological response to the initial treatment from the medical disorder assessment, wherein:
(i) the initial treatment is a standard of care, and the treatment is a more aggressive treatment than the standard of care;
(ii) the treatment is the same as the initial treatment; or
(iii) the treatment is less aggressive than the initial treatment.

19. The method of claim 3, wherein the individual receives the subsequent treatment based on predicting the pathological response to the treatment from the subsequent medical disorder assessment, wherein:
(i) the subsequent treatment is more aggressive than the treatment; or
(ii) the subsequent treatment is less aggressive than the treatment.

20. The method of claim 2, wherein the disorder is diabetes mellitus, and
i) the initial set of clinical data includes at least one of: age, type of diabetes, fasting blood glucose, hemoglobin A1C, or comorbidities; and
ii) the subsequent clinical data includes at least one of: serial fasting blood glucose measurements or hemoglobin A1C measurements; or

21. The method of claim 2, wherein the disorder is sepsis, and
i) and the initial set of clinical data includes at least one of: blood pressure, heart rate, temperature, respiratory rate, oxygenation status, or blood counts; and
ii) the subsequent clinical data includes at least one of: blood culture results, serial blood pressure measurements, heart rate, temperature, respiratory rate, oxygenation status, or blood counts.
